(19) United States
(12) Patent Application Publication
(10) Pub. No.: US 2023/0250048 A1
(43) Pub. Date: Aug. 10, 2023

(54) NITROGEN-CONTAINING COMPOUND, ELECTRONIC ELEMENT AND ELECTRONIC DEVICE

(71) Applicant: Shaanxi Lighte Optoelectronics Material Co., Ltd., Xi'an (CN)

(72) Inventors: Tiantian MA, Xi'an (CN); Lei YANG, Xi'an (CN)

(21) Appl. No.: 18/011,219

(22) PCT Filed: Jul. 8, 2021
(86) PCT No.: PCT/CN2021/105331

§ 371 Date: Dec. 19, 2022

(30) Foreign Application Priority Data
Jul. 9, 2020 (CN) 202010659195.6

(51) Int. Cl. C07C 211/54; H10K 50/15; H10K 50/11; H10K 85/60; C07D 307/91; C07D 209/82; C07D 333/76; C07D 213/74; C07D 405/14; C07D 401/14; C07D 409/12; C07D 405/12; C07D 413/12; H10K 85/40; C07F 7/08

(52) U.S. Cl. CPC C07C 211/54; C07D 209/82; C07D 213/74; C07D 307/91; C07D 333/76; C07D 401/14; C07D 405/12; C07D 405/14; C07D 409/12; C07D 413/12; C07F 7/0812; H10K 50/11; H10K 50/15; H10K 85/40; H10K 85/621; H10K 85/622; H10K 85/626; H10K 85/631; H10K 85/633; H10K 85/636; H10K 85/654; H10K 85/6572; H10K 85/6574; H10K 85/6576; C07B 2200/05

(57) ABSTRACT

The present disclosure belongs to the technical field of organic materials, and provides a nitrogen-containing compound, an electronic element, and an electronic device. The nitrogen-containing compound has a structure shown in formula 1. When the nitrogen-containing compound is applied to an organic electroluminescent device, the service life of the device can be effectively improved while maintaining the luminescence efficiency of the device.

### CROSS REFERENCE TO RELATED APPLICATIONS

The present disclosure claims the priority of Chinese Patent Application No. 202010659195.6, filed on Jul. 9, 2020, and the full content of the above Chinese patent application is incorporated herein as a part of the present disclosure.

### TECHNICAL FIELD

The present disclosure relates to the technical field of organic materials, in particular to a nitrogen-containing compound, an electronic element including the nitrogen-containing compound, and an electronic device including the electronic element.

### BACKGROUND

With the development of electronic technology and the progress of material science, electronic elements for realizing electroluminescence or photoelectric conversion are more and more widely used. Such electronic elements typically include a cathode and an anode which are disposed oppositely, and a functional layer disposed between the cathode and the anode. The functional layer is composed of multiple organic film layers or multiple inorganic film layers, and generally includes an energy conversion layer, a hole transporting layer between the energy conversion layer and the anode, and an electron transporting layer between the energy conversion layer and the cathode.

By taking an organic electroluminescent device as an example, it generally includes an anode, a hole transporting layer, an electroluminescent layer as an energy conversion layer, an electron transporting layer, and a cathode which are stacked sequentially. When the voltage is applied to the cathode and the anode, an electric field is generated between the cathode and the anode, under the effect of the electric field, electrons at the cathode side move towards the electroluminescent layer, and holes at the anode side also move towards the luminescence layer, the electrons and the holes are combined in the electroluminescent layer to form excitons, and the excitons in an excited state release energy outwards, thus causing the electroluminescent layer to emit light outwards.

In the prior art, JP2012167058A discloses a luminescence layer material with 1,8-disubstituted naphthalene as a base structure; and KR1020150006374A discloses a hole transporting layer material with disubstituted naphthalene as a linking group. However, there is still a lot of room for improvement in the structure and performance of this type of material.

The above information of the background of the present disclosure is merely used to enhance an understanding of the context of the present disclosure, and thus it may include information that does not constitute the prior art known to those of ordinary skill in the art.

### SUMMARY

The present disclosure aims to provide a nitrogen-containing compound, an electronic element and an electronic device to improve the performance of the electronic element and the electronic device.

In order to achieve the above inventive purpose, the present disclosure adopts the following technical solutions:

in a first aspect, the present disclosure provides a nitrogen-containing compound, having the structure as shown as formula 1:

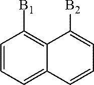

B₁ and B₂ are the same as or different from each other, and are each independently selected from a group represented by formula 1-1, formula 1-2, or formula 1-3, and B₁ and B₂ are not a combination of the formula 1-1 and the formula 1-3:

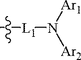

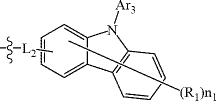

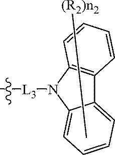

wherein

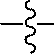

represents chemical bond;
Ar₁, Ar₂, and Ar₃ are the same as or different from each other, and are each independently selected from a substituted or unsubstituted aryl with a total number of carbon atoms being 6 to 30, or a substituted or unsubstituted heteroaryl with a total number of carbon atoms being 3 to 30;
L₁ is selected from a substituted or unsubstituted arylene with a total number of carbon atoms being 6 to 20, or a substituted or unsubstituted heteroarylene with a total number of carbon atoms being 4 to 15;
L₂ and L₃ are the same as or different from each other, and are each independently selected from single bond, a substituted or unsubstituted arylene with a total number of carbon atoms being 6 to 20, or a substituted or unsubstituted heteroarylene with a total number of carbon atoms being 4 to 15;
R₁ and R₂ are the same as or different from each other, and are each independently selected from deuterium, halogen group, cyano, an alkyl with 1 to 10 carbon atoms, a haloalkyl with 1 to 10 carbon atoms, an alkenyl with 2 to 6 carbon atoms, a cycloalkyl with 3 to 10 carbon atoms, a group A, a heteroaryl with 3 to 20 carbon atoms, an alkoxy with 1 to 10 carbon atoms, an alkylthio with 1 to 10 carbon atoms, or a trialkylsilyl with 3 to 12 carbon atoms; and the group A is selected from a substituted or unsubstituted aryl with a total number of carbon atoms being 6 to 20, and substituents in the aryl are each independently selected from deuterium, halogen group, cyano, an alkyl with 1 to 4 carbon atoms, a haloalkyl with 1 to 4 carbon atoms, a cycloalkyl with 3 to 10 carbon atoms, or a trialkylsilyl with 3 to 7 carbon atoms;
m₁ is the number of the substituent R₁, n₁ is selected from 0, 1, 2, 3, 4, 5, 6, or 7, when n₁ is greater than 1, any two R₁ are the same or different, and optionally, any two adjacent R₁ form a ring; and n2 is the number of the substituent R₂, n2 is selected from 0, 1, 2, 3, 4, 5, 6, 7 or 8, when n₂ is greater than 1, any two R₂ are the same or different, and optionally, any two adjacent R₂ form a ring;
substituents in Ar₁, Ar₂, and Ar₃ are the same or different, and are each independently selected from deuterium, halogen group, cyano, an aryl with 6 to 20 carbon atoms, a heteroaryl with 3 to 18 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
substituents in L₁, L₂, and L₃ are the same or different, and are each independently selected from deuterium, halogen group, cyano, an aryl with 6 to 12 carbon atoms, a heteroaryl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
optionally, any two adjacent substituents in the substituents of Ar₁, Ar₂, Ar₃, L₁, L₂ and L₃ form a ring; and,
B₁ and B₂ are not simultaneously

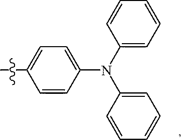

and when B₁ is selected from the structure of the formula 1-3, meanwhile, L₃ is substituted or unsubstituted 1,4-phenylene, the structure of B₂ is different from that of B₁.

In a second aspect, the present disclosure provides an electronic element including an anode and a cathode which are oppositely disposed, and a functional layer disposed between the anode and the cathode; and the functional layer includes the nitrogen-containing compound according to the first aspect.

In a third aspect, the present disclosure provides an electronic device comprising the electronic element according to the second aspect.

The nitrogen-containing compound of the present disclosure takes 1,8-disubstituted naphthalene as a core structure, and triarylamine and/or a group including a carbazole structure are combined in a particular manner as a substituent group for 1,8-disubstituted naphthalene; and in the compound of this structure, triarylamine and the carbazole group both of which have strong hole transport properties are stacked face-to-face at a close distance in space, thus constituting a large conjugated system with a non-planar structure. According to the nitrogen-containing compound of the present disclosure, nitrogen atoms with high chemical activity in the structure are protected on the premise of ensuring the carrier mobility and appropriate molecular orbital energy level of the material, thus improving the stability of the material; and meanwhile, because compound of this type with the non-planar structure has a low intermolecular force, the compound has a low sublimation temperature and evaporation temperature. When such compounds are applied to an organic electroluminescent device, the service life of the device can be effectively improved while maintaining the luminescence efficiency of the device.

### BRIEF DESCRIPTION OF THE DRAWINGS

The above and other features and advantages of the present disclosure will become more apparent by describing in detail exemplary embodiments thereof with reference to the accompanying drawings.

FIG. 1 is a structural schematic diagram of an organic electroluminescent device according to one embodiment of the present disclosure.

FIG. 2 is a structural schematic diagram of a photoelectric conversion device according to one embodiment of the present disclosure.

FIG. 3 is a structural schematic diagram of a first electronic device according to one embodiment of the present disclosure.

FIG. 4 is a structural schematic diagram of a second electronic device according to one embodiment of the present disclosure.

### REFERENCE NUMERALS

100, anode; 200, cathode; 300, functional layer; 310, hole injection layer; 320, hole transporting layer; 321, first hole transporting layer; 322, second hole transporting layer; 330, luminescence layer; 340, electron transporting layer; 350, electron injection layer; 360, photoelectric conversion layer; 400, first electronic device; and 500, second electronic device.

### DETAILED DESCRIPTION

Embodiments will now be described more fully with reference to the accompanying drawings. However, the embodiments can be implemented in various forms and should not be construed as limited to the embodiments set forth here; on the contrary, these embodiments are provided so that the present disclosure will be thorough and complete, and the concept of the embodiments is fully conveyed to those skilled in the art. The described features, structures, or characteristics may be combined in any suitable manner in one or more embodiments. In the following description, many specific details are provided to give a thorough understanding of the embodiments of the present disclosure.

In the drawings, the thickness of regions and layers may be exaggerated for clarity. The same reference signs denote the same or similar structures in the drawings, and thus their detailed description will be omitted.

The described features, structures, or characteristics may be combined in any suitable manner in one or more examples. In the following description, many specific details are provided to give a thorough understanding of the embodiments of the present disclosure. However, those skilled in the art will recognize that the technical solutions of the present disclosure may be practiced without one or more of the specific details, or that other methods, elements, materials, etc. may be employed. In other cases, well-known structures, materials, or operations are not shown or described in detail to avoid obscuring the main technical ideas of the present disclosure.

The present disclosure provides a nitrogen-containing compound, having the structure shown in formula 1:

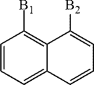

B₁ and B₂ are the same as or different from each other, and are each independently selected from a group represented by formula 1-1, formula 1-2, or formula 1-3, and B₁ and B₂ are not a combination of the formula 1-1 and the formula 1-3:

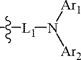

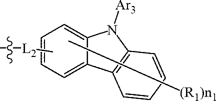

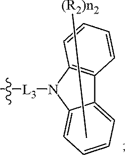

wherein

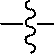

represents chemical bond;
Ar₁, Ar₂, and Ar₃ are the same as or different from each other, and are each independently selected from a substituted or unsubstituted aryl with a total number of carbon atoms being 6 to 30, or a substituted or unsubstituted heteroaryl with a total number of carbon atoms being 3 to 30;
L₁ is selected from a substituted or unsubstituted arylene with a total number of carbon atoms being 6 to 20, or a substituted or unsubstituted heteroarylene with a total number of carbon atoms being 4 to 15;
L₂ and L₃ are the same as or different from each other, and are each independently selected from single bond, a substituted or unsubstituted arylene with a total number of carbon atoms being 6 to 20, or a substituted or unsubstituted heteroarylene with a total number of carbon atoms being 4 to 15;
R₁ and R₂ are the same as or different from each other, and are each independently selected from deuterium, halogen group, cyano, an alkyl with 1 to 10 carbon atoms, a haloalkyl with 1 to 10 carbon atoms, an alkenyl with 2 to 6 carbon atoms, a cycloalkyl with 3 to 10 carbon atoms, a group A, a heteroaryl with 3 to 20 carbon atoms, an alkoxy with 1 to 10 carbon atoms, an alkylthio with 1 to 10 carbon atoms, or a trialkylsilyl with 3 to 12 carbon atoms; and the group A is selected from a substituted or unsubstituted aryl with a total number of carbon atoms being 6 to 20, and substituents in the aryl are each independently selected from deuterium, halogen group, cyano, an alkyl with 1 to 4 carbon atoms, a haloalkyl with 1 to 4 carbon atoms, a cycloalkyl with 3 to 10 carbon atoms, or a trialkylsilyl with 3 to 7 carbon atoms;
n₁ is the number of a substituent R₁, n₁ is selected from 0, 1, 2, 3, 4, 5, 6, or 7, when n₁ is greater than 1, any two R₁ are the same or different, and optionally, any two adjacent R₁ form a ring; and n2 is the number of a substituent R₂, n2 is selected from 0, 1, 2, 3, 4, 5, 6, 7 or 8, when n₂ is greater than 1, any two R₂ are the same or different, and optionally, any two adjacent R₂ form a ring;
substituents in Ar₁, Ar₂, and Ar₃ are the same or different, and are each independently selected from deuterium, halogen group, cyano, an aryl with 6 to 20 carbon atoms, a heteroaryl with 3 to 18 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
substituents in L₁, L₂, and L₃ are the same or different, and are each independently selected from deuterium, halogen group, cyano, an aryl with 6 to 12 carbon atoms, a heteroaryl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
optionally, any two adjacent substituents in the substituents of Ar₁, Ar₂, Ar₃, L₁, L₂ and L₃ form a ring; and,
B₁ and B₂ are not simultaneously

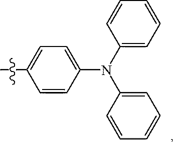

and when B₁ is selected from a structure of the formula 1-3, and meanwhile, L₃ is substituted or unsubstituted 1,4-phenylene, the structure of B₂ is different from that of B₁.

The nitrogen-containing compound of the present disclosure takes 1,8-disubstituted naphthalene as a core structure, and triarylamine and/or a group containing a carbazole structure are combined in a particular manner as a substituent group for 1,8-disubstituted naphthalene; and in the compound of this structure, triarylamine and the carbazole group both of which have strong hole transport properties are stacked face-to-face at a close distance in space, thus constituting a large conjugated system with a non-planar structure. According to the nitrogen-containing compound of the present disclosure, nitrogen atoms with high chemical activity in the structure are protected on the premise of ensuring the carrier mobility and appropriate molecular orbital energy level of the material, thus improving the stability of the material; and meanwhile, because compound of this type having the non-planar structure has a low intermolecular force, the compound has a low sublimation temperature and evaporation temperature. When such compounds are applied to an organic electroluminescent device, the service life of the device can be effectively improved while maintaining the luminescence efficiency of the device.

In order to achieve the inventive purpose of the present disclosure, in the formula 1, B₁ and B₂ are not a combination of the formula 1-1 and the formula 1-3, which means that when one of B₁ and B₂ is the group represented by the formula 1-1, the other cannot be the group represented by the formula 1-3, but the other can be, for example, selected from the group represented by the formula 1-1 or the formula 1-2.

In the present disclosure, a total number of carbon atoms of a substituted group refers to the number of all carbon atoms. For example, if Ar₁ is selected from a substituted aryl with a total number of carbon atoms being 20, the number of all carbon atoms of the aryl and substituents on the aryl is 20.

In the present disclosure, the used descriptions manners “each...is independently”, “...is respectively and independently” and “...is independently selected from” can be interchanged, which should be understood in a broad sense. And they may mean that specific options expressed by a same symbol in different groups do not influence each other, or that specific options expressed by a same symbol in a same group do not influence each other. For example, the meaning of”

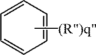

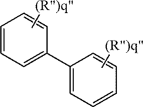

, wherein each q″ is independently 0, 1, 2 or 3 and each R″ is independently selected from hydrogen, deuterium, fluorine, or chlorine” is as follows: formula Q-1 represents that there are substituents R″ in an amount of q″ on the benzene ring, each R″ can be the same or different, and options of each R″ do not influence each other; and formula Q-2 represents that there are substituents R″ in an amount of q″ on each of benzene ring in the biphenyl, the number q″ of the substituents R″ on the two benzene rings can be the same or different, each R″ can be the same or different, and options of each R″ do not influence each other.

In the present disclosure, the term “optional” or “optionally” means that the subsequently described event or circumstance may but not need to occur, and that description includes occasions where the event or circumstance occurs or does not occur. For example, “optionally, any two adjacent R₁ form a ring”, which means that any two adjacent R₁ may, but not need to, form a ring, including a scenario where any two adjacent R₁ form a ring and a scenario where any two adjacent R₁ do not form a ring.

The involved situation that adjacent groups “form a ring” includes the adjacent groups may be connected by a single bond to form a ring together with the atom to which they are commonly connected, or the adjacent groups are fused together with the atoms to which they are respectively connected to form a ring. The ring formed by the adjacent groups can, for example, be a 5- to 13-membered saturated or unsaturated ring. The ring formed by the adjacent groups together with the atom to which they are commonly connected may, for example, be a fluorene ring, cyclohexane, or cyclopentane, and the ring formed by fusing the adjacent groups together with the atoms to which they are respectively connected may, for example, be a benzene ring or a naphthalene ring.

In the present disclosure, aryl refers to an optional functional group or substituent derived from an aromatic carbocyclic ring. The aryl can be a monocyclic aryl (e.g., phenyl) or a polycyclic aryl, in other words, the aryl can be a monocyclic aryl, a fused cyclic aryl, two or more monocyclic aryls which are conjugatedly linked by carbon-carbon bonds, a monocyclic aryl and a fused cyclic aryl which are conjugatedly linked by a carbon-carbon bond, and two or more fused cyclic aryls which are conjugatedly linked by carbon-carbon bonds. That is, unless specified otherwise, two or more aromatic groups conjugatedly linked by carbon-carbon bonds can also be regarded as aryl of the present disclosure. The fused cyclic aryl may, for example, include a bicyclic fused cyclic aryl (e.g., naphthyl), a tricyclic fused cyclic aryl (e.g., phenanthryl, fluorenyl, and anthryl), and the like. The aryl does not contain heteroatoms such as B, N, O, S, P, Si and the like. It should be noted that in the present disclosure, biphenyl and 9,9-dimethylfluorenyl are both regarded as aryl. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, spirobifluorenyl, and the like. In the present disclosure, involved arylene refers to a divalent group formed by further loss of one hydrogen atom of the aryl.

In the present disclosure, substituted aryl can be that one or two or more hydrogen atoms in the aryl are substituted with groups such as deuterium atom, halogen group, -CN, aryl, heteroaryl, trialkylsilyl, alkyl, cycloalkyl, alkoxy, alkylthio, and the like. Specific examples of aryl substituted by heteroaryl include, but are not limited to, phenyl substituted by dibenzofuranyl, phenyl substituted by dibenzothienyl, phenyl substituted by pyridyl, and the like. It should be understood that the total number of carbon atoms of the substituted aryl refers to the total number of carbon atoms of the aryl and substituents on the aryl. Optionally, any two adjacent substituents may form a ring, and may, for example, form a ring by single bond, or may be fused to form a ring.

In the present disclosure, heteroaryl refers to a monovalent aromatic ring containing at least one heteroatom in the ring or its derivative, and the heteroatom may be at least one of B, O, N, P, Si, and S. The heteroaryl can be a monocyclic heteroaryl or a polycyclic heteroaryl, in other words, the heteroaryl can be a single aromatic ring system or a plurality of aromatic ring systems conjugatedly linked via carbon-carbon bonds, and any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to this. Thienyl, furyl, phenanthrolinyl, etc. are heteroaryl of the single aromatic ring system, and N-phenylcarbazolyl, and N-pyridylcarbazolyl are heteroaryl of the plurality of aromatic ring systems conjugatedly linked via carbon-carbon bonds. In addition, benzofuranyl, benzopyridyl, and the like belong to bicyclic fused heteroaryl, and dibenzofuranyl, dibenzothienyl, carbazolyl, and the like belong to tricyclic fused heteroaryl. In the present disclosure, involved heteroarylene refers to a divalent group formed by further loss of one hydrogen atom of the heteroaryl.

In the present disclosure, substituted heteroaryl may be that one or two or more hydrogen atoms in the heteroaryl are substituted with groups such as deuterium atom, halogen group, -CN, aryl, heteroaryl, trialkylsilyl, alkyl, cycloalkyl, alkoxy, alkylthio, and the like. Specific examples of heteroaryl substituted by aryl include, but are not limited to, dibenzofuranyl substituted by phenyl, dibenzothienyl substituted by phenyl, pyridyl substituted by phenyl, and the like. It should be understood that the total number of carbon atoms of the substituted heteroaryl refers to the total number of carbon atoms of the heteroaryl and substituents on the heteroaryl. Optionally, any two adjacent substituents may form a ring, and may, for example, form a ring by single bond, or may be fused to form a ring.

In the present disclosure, the number of ring-forming carbon atoms refers to the number of carbon atoms located on an aromatic ring (an aryl ring or a heteroaromatic ring). For example, phenyl has 6 ring-forming carbon atoms, naphthyl has 10 ring-forming carbon atoms, biphenyl has 12 ring-forming carbon atoms, dibenzofuranyl has 12 ring-forming carbon atoms, and N-phenylcarbazolyl has 18 ring-forming carbon atoms. It should be noted that in the present disclosure, the number of carbon atoms of aryl and heteroaryl as substituents is also considered to be in the number of ring-forming carbon atoms, for example,

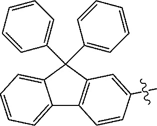

has 25 ring-forming carbon atoms, and

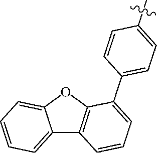

has 18 ring-forming carbon atoms.

In the present disclosure, an nonlocalized connecting bond refers to a single bond “

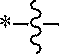

”extending from a ring system, which means that one end of the connecting bond can be connected with any position in the ring system through which the bond penetrates, and the other end of the connecting bond is connected with the rest part of a compound molecule.

For example, as shown in the following formula (f), naphthyl represented by the formula (f) is connected to other positions of a molecule through two nonlocalized connecting bonds penetrating a dicyclic ring, and its meaning includes any one possible connecting mode represented by formulae (f-1)-(f-10).

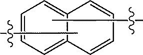

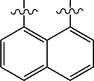

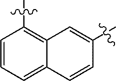

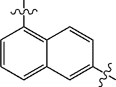

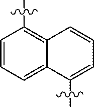

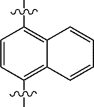

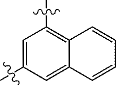

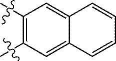

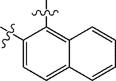

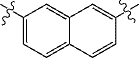

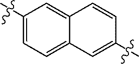

For another embodiment, as shown in the following formula (X′), phenanthryl represented by the formula (X′) is connected with other positions of a molecule through one nonlocalized connecting bond extending from the middle of a benzene ring on one side, and its meaning includes any possible connecting mode represented by formulae (X′-1)-(X′-4).

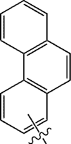

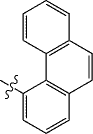

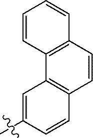

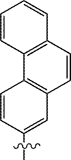

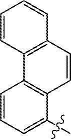

An nonlocalized substituent in the present disclosure refers to a substituent connected through a single bond extending from the center of a ring system, which means that the substituent can be connected to any possible position in the ring system. For example, as shown in the following formula (Y), a substituent R′ represented by the formula (Y) is connected with a quinoline ring through one nonlocalized connecting bond, and its meaning includes any one possible connecting mode represented by formulae (Y-1)-(Y-7):

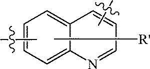

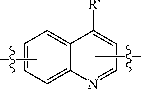

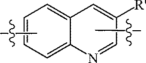

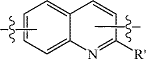

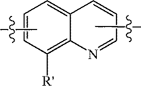

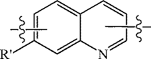

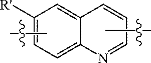

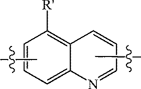

In the present disclosure, cycloalkyl with 3 to 10 carbon atoms can be used as a substituent for aryl and heteroaryl, and specific examples include, but are not limited to, cyclopentyl, cyclohexyl, adamantyl, and the like.

In the present disclosure, alkyl with 1 to 10 carbon atoms includes straight-chain alkyl with 1 to 10 carbon atoms and branched-chain alkyl with 3 to 10 carbon atoms, and the number of carbon atoms can be 1, 2, 3, 4, 5, 6, 7, 8, 9, or 10, and specific examples of alkyl can include, but are not limited to, methyl, ethyl, n-propyl, isopropyl, n-butyl, tert-butyl, n-pentyl, n-hexyl, n-heptyl, n-octyl, and the like.

In the present disclosure, halogen includes fluorine, chlorine, bromine, and iodine.

In the present disclosure, the number of carbon atoms of alkoxy with 1 to 10 carbon atoms can, for example, be 1, 2, 3, 4, 5, 6, 7, 8, 9, or 10, and specific examples of alkoxy include, but are not limited to, methoxy, ethoxy, n-propoxy, and the like.

In the present disclosure, the number of carbon atoms of aryl with 6 to 20 carbon atoms may, for example, be 6 (e.g., phenyl), 10 (e.g., naphthyl), 12 (e.g., biphenyl), 15 (9,9-dimethylfluorenyl), 18 (e.g., terphenyl), or the like. Specific examples of the aryl with 6 to 12 carbon atoms include, but are not limited to, phenyl, naphthyl, and biphenyl.

In the present disclosure, haloalkyl can, for example, be fluoroalkyl, and the number of carbon atoms can, for example, be 1, 2, 3, 4, 5, 6, 7, 8, 9, or 10, and specific examples include, but not limited to, trifluoromethyl.

In the present disclosure, specific examples of trialkylsilyl include, but are not limited to, trimethylsilyl, triethylsilyl, and the like.

In some embodiments, the structure of the nitrogen-containing compound can be shown in formula 11,

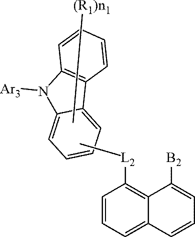

wherein the structure of B₂ is selected from the formula 1-1, the formula 1-2, or the formula 1-3.

In the present disclosure, the nitrogen-containing compound can be selected from the group consisting of formulae A to E as below:

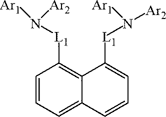

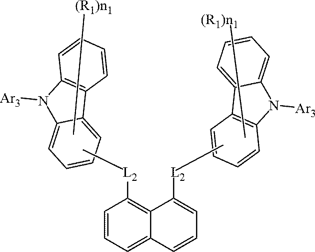

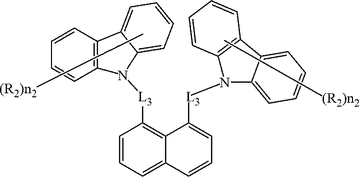

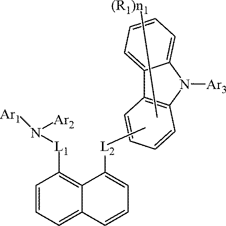

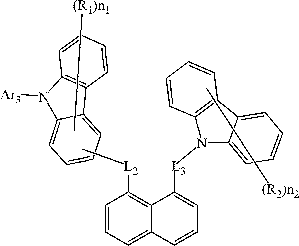

In the formula A, both Ar₁ may be the same or different, and both Ar₂ may also be the same or different. In the formula B, both Ar₃ may be the same or different, both L₂ may be the same or different, and when a plurality of R₁ are present, R₁ and n₁ may also be the same or different. In the formula C, both L₃ may be the same or different, and when a plurality of R₂ are present, R₂ and n₂ may also be the same or different. In order to achieve the inventive purpose of the present disclosure, in the formula A, both

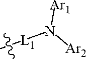

are not simultaneously

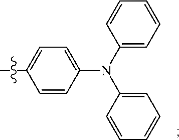

and in the formula C, when one L₃ is substituted or unsubstituted 1,4-phenylene (a structure of 1,4-phenylene is

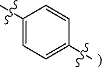

, specific structures of both

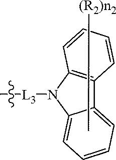

are different.

In one exemplary embodiment, for the two

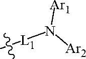

in the formula A, one

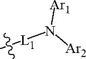

may be

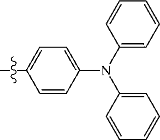

, and meanwhile in the other

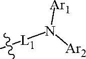

, Ar₂ may be selected from a substituted or unsubstituted aryl with a total number of carbon atoms being 7 to 30, or a substituted or unsubstituted heteroaryl with a total number of carbon atoms being 3 to 30. When the Ar₂ is selected from a substituted or unsubstituted aryl, the number of carbon atoms may be 7, 8, 9, 10, 11, 12, 13, 14, 15, 16, 17, 18, 19, 20, 21, 22, 23, 24, 25, 26, 27, 28, 29, or 30; and when the Ar₂ is selected from a substituted or unsubstituted heteroaryl, the number of carbon atoms may be 3, 4, 5, 6, 7, 8, 9, 10, 11, 12, 13, 14, 15, 16, 17, 18, 19, 20, 21, 22, 23, 24, 25, 26, 27, 28, 29, or 30.

In the present disclosure, optionally, R₁ and R₂ are the same as or different from each other, and are each independently selected from deuterium, fluorine, cyano, an alkyl with 1 to 4 carbon atoms, a haloalkyl with 1 to 4 carbon atoms, a cycloalkyl with 5 to 10 carbon atoms, a group B, a heteroaryl with 3 to 18 carbon atoms, an alkoxy with 1 to 4 carbon atoms, an alkylthio with 1 to 4 carbon atoms, or a trialkylsilyl with 3 to 7 carbon atoms. The group B is selected from a substituted or unsubstituted aryl with a total number of carbon atoms being 6 to 15, and substituents in the aryl are each independently selected from deuterium, fluorine, cyano, an alkyl with 1 to 4 carbon atoms, a fluoroalkyl with 1 to 4 carbon atoms, or a trialkylsilyl with 3 to 7 carbon atoms. For example, R₁ and R₂ are each independently selected from deuterium, fluorine, cyano, methyl, tert-butyl, trifluoromethyl, trimethylsilyl, cyclopentyl, cyclohexyl, adamantyl, phenyl, naphthyl, biphenyl, phenyl substituted by trifluoromethyl, methyl, tert-butyl, cyano, fluorine or deuterium, and the like.

Optionally, any two adjacent R₁ may be fused to a ring, for example a benzene ring. Optionally, any two adjacent R₂ may be fused to a ring, for example a benzene ring.

In the present disclosure, optionally, Ar₁, Ar₂ and Ar₃ are the same as or different from each other, and are each independently selected from a substituted or unsubstituted aryl with a total number of carbon atoms being 6 to 25, or a substituted or unsubstituted heteroaryl with a total number of carbon atoms being 5 to 25.

Optionally, substituents in Ar₁, Ar₂, and Ar₃ are each independently selected from deuterium, fluorine, cyano, an aryl with 6 to 15 carbon atoms, a heteroaryl with 5 to 12 carbon atoms, an alkyl with 1 to 4 carbon atoms, a haloalkyl with 1 to 4 carbon atoms, a cycloalkyl with 5 to 10 carbon atoms, an alkoxy with 1 to 4 carbon atoms, an alkylthio with 1 to 4 carbon atoms, or a trialkylsilyl with 3 to 7 carbon atoms. Optionally, any two adjacent substituents form a 5- to 13-membered saturated or unsaturated ring.

Optionally, the substituents in Ar₁, Ar₂, and Ar₃ are each independently selected from deuterium, fluorine, cyano, phenyl, naphthyl, biphenyl, phenanthryl, anthryl, dimethylfluorenyl, pyridyl, quinolyl, dibenzofuranyl, dibenzothienyl, carbazolyl, methyl, ethyl, isopropyl, tert-butyl, trifluoromethyl, cyclopentyl, cyclohexyl, adamantyl, trimethylsilyl, and methylthio. Optionally, any two adjacent substituents form a fluorene ring, cyclopentane or cyclohexane.

Also optionally, Ar₁, Ar₂, and Ar₃ are the same as or different from each other, and are each independently selected from a substituted or unsubstituted aryl with 6 to 25 ring-forming carbon atoms, or a substituted or unsubstituted heteroaryl with 5 to 25 ring-forming carbon atoms. The number of ring-forming carbon atoms of substituted or unsubstituted aryl is, for example, 6, 10, 12, 13, 18, 21, 25, or the like, and the number of ring-forming carbon atoms of substituted or unsubstituted heteroaryl is, for example, 5, 6, 7, 8, 9, 10, 12, 18, 20, 24, 25, or the like.

In some embodiments, Ar₁, Ar₂ and Ar₃ are the same as or different from each other, and are each independently selected from the group consisting of groups represented by formulae i-1 to i-15:

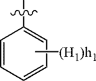

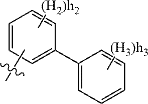

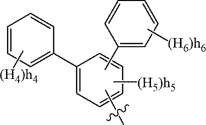

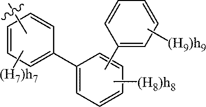

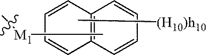

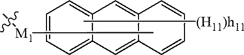

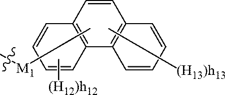

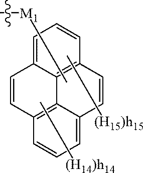

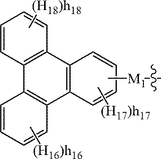

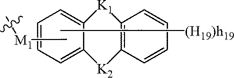

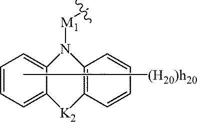

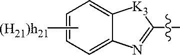

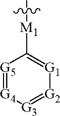

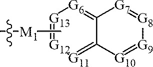

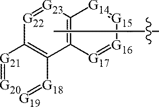

wherein M₁ is selected from single bond or

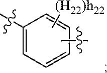

G₁ to G₅ are each independently selected from N or C(F₁), and at least one of G₁ to G₅ is selected from N; and when two or more of G₁ to G₅ are selected from C(F₁), any two F₁ are the same or different;
G₆ to G₁₃ are each independently selected from N or C(F₂), and at least one of G₆ to G₁₃ is selected from N; and when two or more of G₆ to G₁₃ are selected from C(F₂), any two F₂ are the same or different;
G₁₄ to G₂₃ are each independently selected from N or C(F₃), and at least one of G₁₄ to G₂₃ is selected from N; and when two or more of G₁₄ to G₂₃ are selected from C(F₃), any two F₃ are the same or different;
H₁ is selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or an alkylthio with 1 to 10 carbon atoms;
H₂ to H₉, and H₂₂ are each independently selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a heteroaryl with 3 to 18 carbon atoms;
H₁₀ to H₂₁, and F₁ to F₄ are each independently selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, an aryl with 6 to 18 carbon atoms, or a heteroaryl with 3 to 18 carbon atoms;
h₁ to h₂₂ are represented by hₖ, H₁ to H₂₂ are represented by Hₖ, k is a variable, and represents any integer from 1 to 22, and hₖ represents the number of a substituent Hₖ; when k is selected from 5 or 17, hₖ is selected from 1, 2 or 3; when k is selected from 2, 7, 8, 12, 15, 16, 18, 21 or 22, hₖ is selected from 1, 2, 3 or 4; when k is selected from 1, 3, 4, 6, 9 or 14, hₖ is selected from 1, 2, 3, 4 or 5; when k is 13, hₖ is selected from 1, 2, 3, 4, 5, or 6; when k is selected from 10 or 19, hₖ is selected from 1, 2, 3, 4, 5, 6 or 7; when k is 20, hₖ is selected from 1, 2, 3, 4, 5, 6, 7, or 8; when k is 11, hₖ is selected from 1, 2, 3, 4, 5, 6, 7, 8 or 9; and when hₖ is greater than 1, any two Hₖ are the same or different;
K₁ is selected from O, S, Se, N(H₂₃), C(H₂₄H₂₅), or Si(H₂₄H₂₅); wherein H₂₃, H₂₄, and H₂₅ are each independently selected from an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₄ and the H₂₅ are connected to each other to form a 5- to 13-membered saturated or unsaturated ring together with the atom to which they are commonly connected;
K₂ is selected from single bond, O, S, Se, N(H₂₆), C(H₂₇H₂₈), or Si(H₂₇H₂₈); wherein H₂₆, H₂₇, and H₂₈ are each independently selected from an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₇ and the H₂₈ are connected to each other to form a 5- to 13-membered saturated or unsaturated ring together with the atom to which they are commonly connected;
and K₃ represents O or S.

In the present disclosure, in the formulae i-10 and i-11, when K₂ represents single bond, specific structures of the formulae i-10 and i-11 are shown below, respectively:

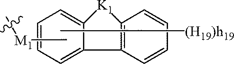

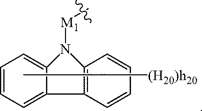

In the present disclosure, in both groups of the H₂₄ and the H₂₅, and the H₂₇ and the H₂₈, a ring formed by connecting two groups in each group with each other may be a 5- to 13-membered saturated or unsaturated ring. Optionally, in both groups of the H₂₄ and the H₂₅, and the H₂₇ and the H₂₈, the ring formed by connecting two groups in each group with each other may be a 5- to 13-membered saturated aliphatic or aromatic ring. According to one embodiment, the both groups of the H₂₄ and the H₂₅, and the H₂₇ and the H₂₈ may respectively form a 5- to 8-membered saturated aliphatic monocyclic ring or form a 10- to 13-membered aromatic ring. For example, in the formula i-10, when K₂ and M₁ are both single bond, h₁₉=7, H₁₉ is hydrogen, K₁ is C(H₂₄H₂₅), and H₂₄ and H₂₅ are connected to each other to form a 5-membered saturated aliphatic monocyclic ring together with the atom to which they are commonly connected, thus the formula i-10 is

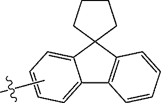

; likewise, the formula i-10 may also be

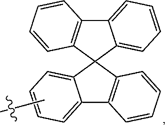

that is, H₂₄ and H₂₅ are connected to each other to form a 13-membered aromatic ring together with the atom to which they are commonly connected.

In the formulae i-13 to i-15, F₂ to F₄ may be represented by Fⱼ, wherein j is a variable and represents 2, 3, or 4. For example, when j is 2, Jⱼ refers to J₂. It should be understood that Fⱼ in C(Fⱼ) is not present when an nonlocalized connecting bond is connected to C(Fⱼ). For example, in the formula i-13, when

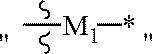

is connected to G₁₂, G₁₂ can only represent a C atom, i.e., a structure of the formula i-13 is specifically:

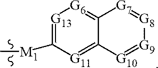

Similarly, in formula j-4 below referring to L₁ to L₃, when

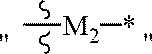

is connected to Q (e.g. Q₁) of each C-containing group (C(F₄)), Q represents a C atom.

Optionally, Ar₁, Ar₂ and Ar₃ are the same as or different from each other, and are each independently selected from a substituted or unsubstituted group Z, and the unsubstituted group Z is selected from the group consisting of:

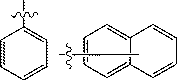

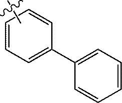

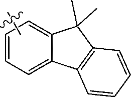

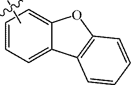

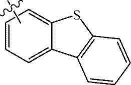

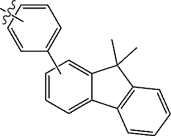

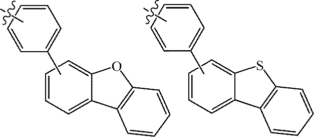

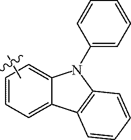

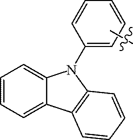

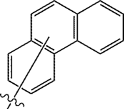

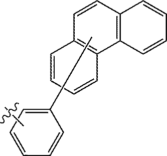

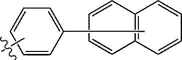

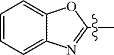

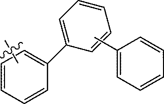

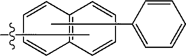

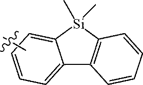

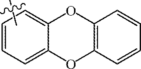

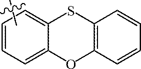

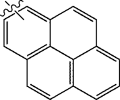

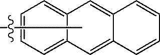

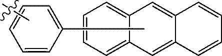

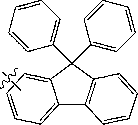

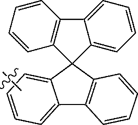

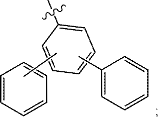

and the substituted group Z has one or two or more substituents, and the substituents are each selected from deuterium, cyano, fluorine, an alkyl with 1 to 4 carbon atoms, a cycloalkyl with 5 to 10 carbon atoms, a haloalkyl with 1 to 4 carbon atoms, or a trialkylsilyl with 3 to 7 carbon atoms; and when the number of the substituents is greater than 1, the substituents are the same or different.

Optionally, Ar₁, Ar₂ and Ar₃ are the same as or different from each other, and are each independently selected from the group consisting of:

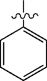

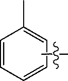

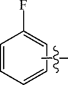

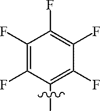

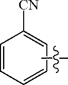

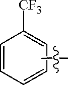

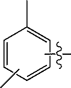

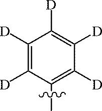

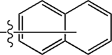

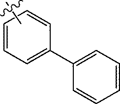

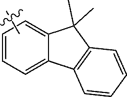

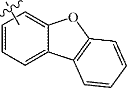

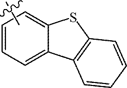

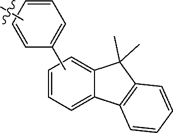

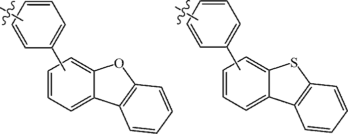

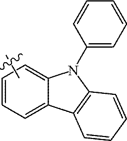

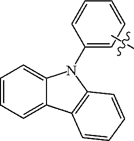

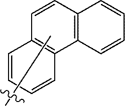

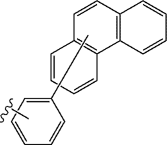

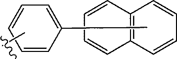

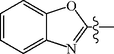

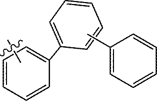

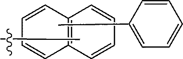

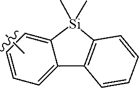

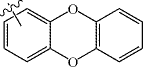

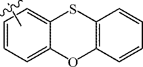

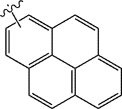

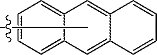

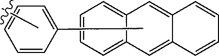

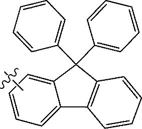

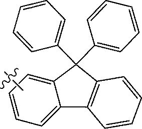

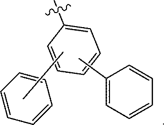

Further optionally, Ar₁, Ar₂ and Ar₃ are the same as or different from each other, and are each independently selected from the group consisting of:

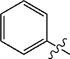

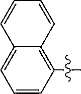

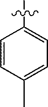

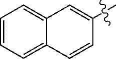

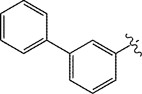

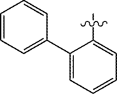

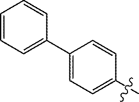

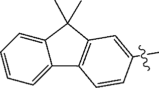

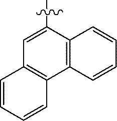

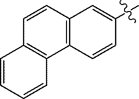

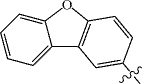

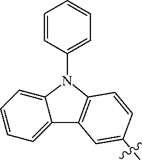

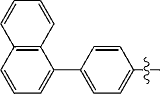

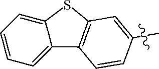

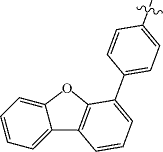

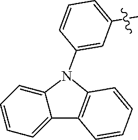

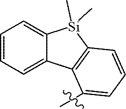

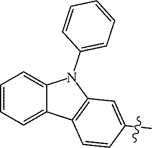

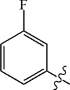

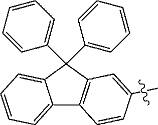

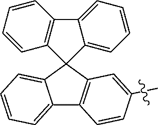

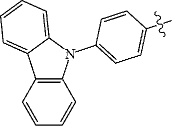

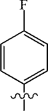

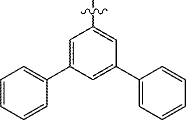

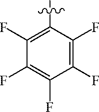

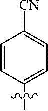

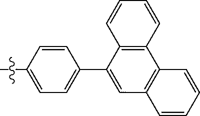

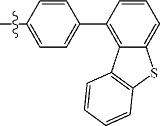

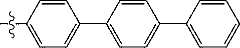

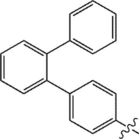

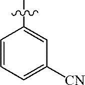

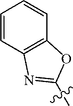

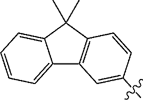

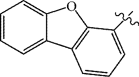

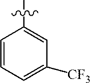

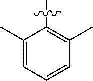

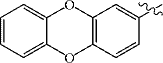

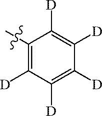

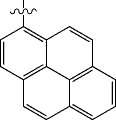

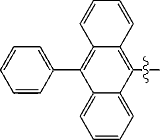

In the present disclosure, optionally, L₁ and L₃ are the same as or different from each other, and are each independently selected from a substituted or unsubstituted arylene with a total number of carbon atoms being 6 to 12, or a substituted or unsubstituted heteroarylene with a total number of carbon atoms being 5 to 12.

Also optionally, L₁ and L₃ are the same as or different from each other, and are each independently selected from a substituted or unsubstituted aryl with 6 to 12 ring-forming carbon atoms, or a substituted or unsubstituted heteroaryl with 5 to 12 ring-forming carbon atoms. The number of ring-forming carbon atoms of substituted or unsubstituted aryl is, for example, 6, 10, 12, or the like, and the number of ring-forming carbon atoms of substituted or unsubstituted heteroaryl is, for example, 5, 6, 7, 8, 9, 10, or 12.

Optionally, L₂ is selected from single bond, a substituted or unsubstituted arylene with a total number of carbon atoms being 6 to 12, or a substituted or unsubstituted heteroarylene with a total number of carbon atoms being 5 to 12.

Also optionally, L₂ is selected from single bond, a substituted or unsubstituted arylene with 6 to 12 ring-forming carbon atoms, or a substituted or unsubstituted heteroarylene with 5 to 12 ring-forming carbon atoms. The number of ring-forming carbon atoms of substituted or unsubstituted arylene is, for example, 6, 10, 12, or the like, and the number of ring-forming carbon atoms of substituted or unsubstituted heteroarylene is, for example, 5, 6, 7, 8, 9, 10, 12, or the like.

Optionally, substituents in L₁, L₂ and L₃ are the same or different, and are each independently selected from deuterium, fluorine, cyano, phenyl, pyridyl, an alkyl with 1 to 4 carbon atoms, a haloalkyl with 1 to 4 carbon atoms, or a trialkylsilyl with 3 to 7 carbon atoms.

Further optionally, the substituents in L₁, L₂ and L₃ are each independently selected from deuterium, fluorine, cyano, phenyl, pyridyl, methyl, ethyl, isopropyl, tert-butyl, trifluoromethyl or trimethylsilyl.

In some embodiments, L₁, L₂ and L₃ are the same as or different from each other, and are each independently selected from the group consisting of groups represented by formulae j-1 to j-4 below:

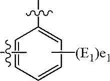

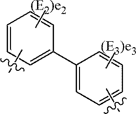

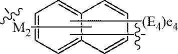

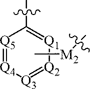

wherein M₂ is selected from single bond or

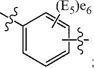

Q₁ to Q₅ are each independently selected from N or C(F₄), and at least one of Q₁ to Q₅ is selected from N; and when two or more of Q₁ to Q₅ are selected from C(F₄), any two F₄ are the same or different;
E₁ to Es, and F₄ are each independently selected from hydrogen, deuterium, fluorine, chlorine, bromine, cyano, a heteroaryl with 3 to 10 carbon atoms, an aryl with 6 to 12 carbon atoms, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or a alkylthio with 1 to 10 carbon atoms;
and E₁ to E₅ are represented by Eᵣ, and e₁ to e₅ are represented by eᵣ, wherein r represents a variable and is selected from any integer from 1 to 5; when r is selected from 1, 2, 3 or 5, eᵣ is selected from 1, 2, 3 or 4; when r is 4, eᵣ is selected from 1, 2, 3, 4, 5, or 6; and when eᵣ is greater than 1, any two Eᵣ are the same or different.
Optionally, L₁, L₂ and L₃ are the same as or different from each other, and are each independently selected from a substituted or unsubstituted group V, and the unsubstituted group V is selected from the group consisting of:

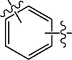

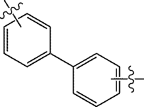

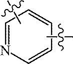

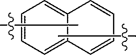

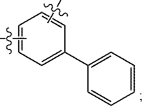

and the substituted group V has one or two or more substituents, and the substituents are respectively and independently selected from deuterium, cyano, fluorine, an alkyl with 1 to 4 carbon atoms, a cycloalkyl with 5 to 10 carbon atoms, a haloalkyl with 1 to 4 carbon atoms, or a trialkylsilyl with 3 to 7 carbon atoms; and when the number of the substituents is greater than 1, the substituents are the same or different.
Optionally, L₁, L₂ and L₃ are the same as or different from each other, and are each independently selected from a substituted or unsubstituted phenylene, a substituted or unsubstituted biphenylene, a substituted or unsubstituted pyridylene, or a substituted or unsubstituted naphthylene, and the substituent is phenyl.

According to one exemplary embodiment, L₁, L₂ and L₃ are the same as or different from each other, and are each independently selected from the group consisting of:

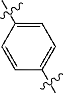

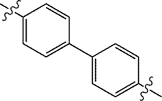

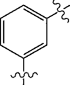

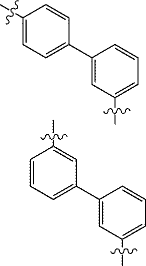

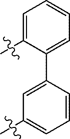

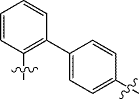

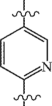

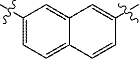

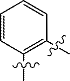

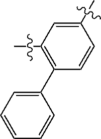

In one embodiment, the nitrogen-containing compound has a structure as shown in the formula A or the formula D. In this embodiment, the nitrogen-containing compound may be used as a hole transporting layer material of an electronic element, for example as a first hole transporting layer material and/or a second hole transporting layer (also called an electron blocking layer) material of an organic electroluminescent device.

Preferably, Ar₁, Ar2, and Ar₃ are the same as or different from each other, and are each independently selected from a substituted or unsubstituted aryl with a total number of carbon atoms being 6 to 25, or a substituted or unsubstituted heteroaryl with a total number of carbon atoms being 5 to 20.

Preferably, L₁ is selected from a substituted or unsubstituted arylene with a total number of carbon atoms being 6 to 12; and L₂ is selected from single bond, or a substituted or unsubstituted arylene with a total number of carbon atoms being 6 to 12.

In another embodiment, the nitrogen-containing compound has a structure as shown in the formula B, the formula C, or the formula E. In this embodiment, the nitrogen-containing compound may be used as a luminescence layer material of an electronic element, for example a luminescence layer host material of an organic electroluminescent device.

Preferably, Ar₃ is selected from a substituted or unsubstituted aryl with a total number of carbon atoms being 6 to 20, or a substituted or unsubstituted heteroaryl with a total number of carbon atoms being 5 to 20.

Preferably, L₂ is selected from single bond, or a substituted or unsubstituted arylene with a total number of carbon atoms being 6 to 12.

Preferably, L₃ is selected from a substituted or unsubstituted arylene with a total number of carbon atoms being 6 to 12.

In one embodiment, the nitrogen-containing compound has a structure as shown in the formula A. Preferably, Ar₁ is selected from a substituted or unsubstituted aryl with 6 to 20 ring-forming carbon atoms, and Ar₂ is selected from a substituted or unsubstituted aryl with 10 to 25 ring-forming carbon atoms, or a substituted or unsubstituted heteroaryl with 7 to 20 ring-forming carbon atoms. In this case, the nitrogen-containing compound may further increase the overall performance of the device, in particular the service life of the device.

In another specific embodiment, the nitrogen-containing compound has a structure as shown in the formula C. Preferably, at least one L₃ in the two L₃ is selected from a substituted or unsubstituted arylene with 10 to 20 ring-forming carbon atoms. In this case, the nitrogen-containing compound may further increase the overall performance of the device, in particular the service life of the device.

Optionally, the nitrogen-containing compound is selected from the group consisting of the following compounds:

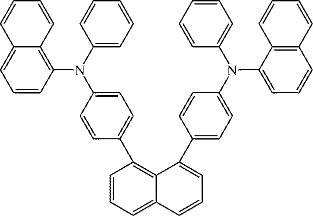 [SMILES: c1ccc(N(c2ccc(-c3cccc4cccc(-c5ccc(N(c6ccccc6)c6cccc7ccccc67)cc5)c34)cc2)c2cccc3ccccc23)cc1]

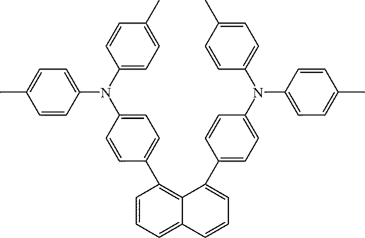

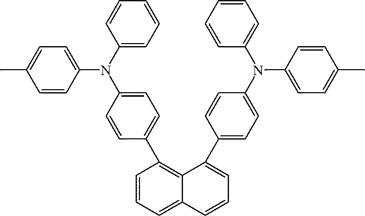

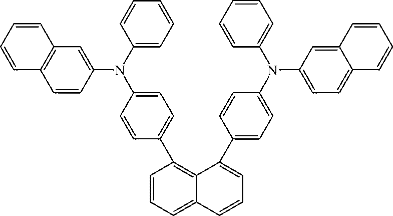 [SMILES: c1ccc(N(c2ccc(-c3cccc4cccc(-c5ccc(N(c6ccccc6)c6ccc7ccccc7c6)cc5)c34)cc2)c2ccc3ccccc3c2)cc1]

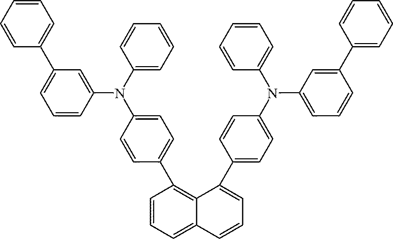 [SMILES: c1ccc(-c2cccc(N(c3ccccc3)c3ccc(-c4cccc5cccc(-c6ccc(N(c7ccccc7)c7cccc(-c8ccccc8)c7)cc6)c45)cc3)c2)cc1]

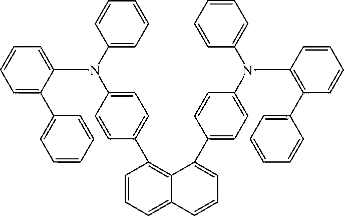 [SMILES: c1ccc(-c2ccccc2N(c2ccccc2)c2ccc(-c3cccc4cccc(-c5ccc(N(c6ccccc6)c6ccccc6-c6ccccc6)cc5)c34)cc2)cc1]

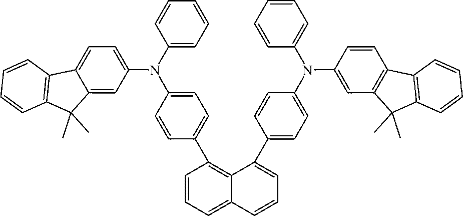 [SMILES: CC1(C)c2ccccc2-c2ccc(N(c3ccccc3)c3ccc(-c4cccc5cccc(-c6ccc(N(c7ccccc7)c7ccc8c(c7)C(C)(C)c7ccccc7-8)cc6)c45)cc3)cc21]

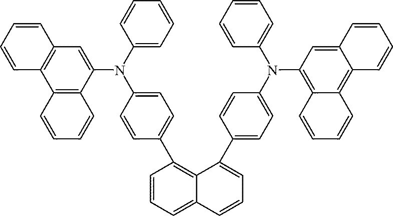 [SMILES: c1ccc(N(c2ccc(-c3cccc4cccc(-c5ccc(N(c6ccccc6)c6cc7ccccc7c7ccccc67)cc5)c34)cc2)c2cc3ccccc3c3ccccc23)cc1]

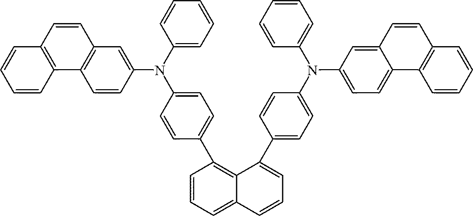 [SMILES: c1ccc(N(c2ccc(-c3cccc4cccc(-c5ccc(N(c6ccccc6)c6ccc7c(ccc8ccccc87)c6)cc5)c34)cc2)c2ccc3c(ccc4ccccc43)c2)cc1]

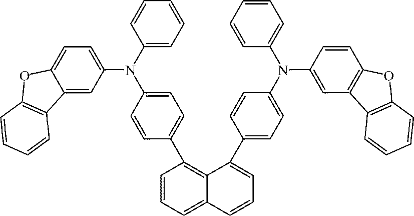 [SMILES: c1ccc(N(c2ccc(-c3cccc4cccc(-c5ccc(N(c6ccccc6)c6ccc7oc8ccccc8c7c6)cc5)c34)cc2)c2ccc3oc4ccccc4c3c2)cc1]

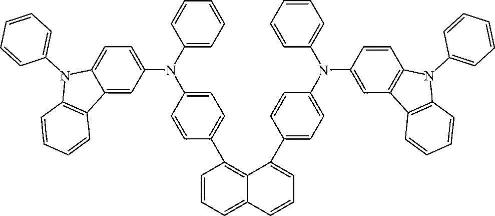 [SMILES: c1ccc(N(c2ccc(-c3cccc4cccc(-c5ccc(N(c6ccccc6)c6ccc7c(c6)c6ccccc6n7-c6ccccc6)cc5)c34)cc2)c2ccc3c(c2)c2ccccc2n3-c2ccccc2)cc1]

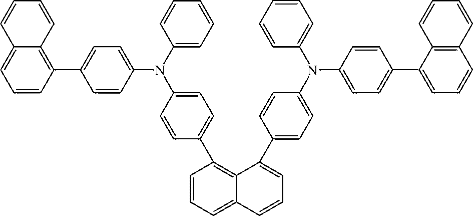 [SMILES: c1ccc(N(c2ccc(-c3cccc4ccccc34)cc2)c2ccc(-c3cccc4cccc(-c5ccc(N(c6ccccc6)c6ccc(-c7cccc8ccccc78)cc6)cc5)c34)cc2)cc1]

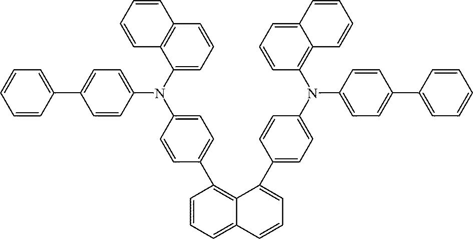 [SMILES: c1ccc(-c2ccc(N(c3ccc(-c4cccc5cccc(-c6ccc(N(c7ccc(-c8ccccc8)cc7)c7cccc8ccccc78)cc6)c45)cc3)c3cccc4ccccc34)cc2)cc1]

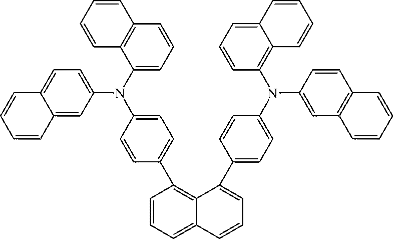 [SMILES: c1ccc2cc(N(c3ccc(-c4cccc5cccc(-c6ccc(N(c7ccc8ccccc8c7)c7cccc8ccccc78)cc6)c45)cc3)c3cccc4ccccc34)ccc2c1]

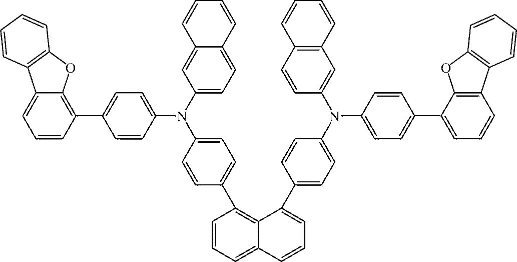 [SMILES: c1ccc2cc(N(c3ccc(-c4cccc5c4oc4ccccc45)cc3)c3ccc(-c4cccc5cccc(-c6ccc(N(c7ccc(-c8cccc9c8oc8ccccc89)cc7)c7ccc8ccccc8c7)cc6)c45)cc3)ccc2c1]

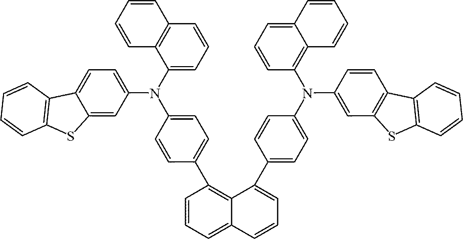 [SMILES: c1ccc2c(N(c3ccc(-c4cccc5cccc(-c6ccc(N(c7ccc8c(c7)sc7ccccc78)c7cccc8ccccc78)cc6)c45)cc3)c3ccc4c(c3)sc3ccccc34)cccc2c1]

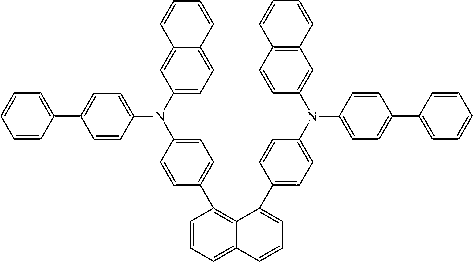 [SMILES: c1ccc(-c2ccc(N(c3ccc(-c4cccc5cccc(-c6ccc(N(c7ccc(-c8ccccc8)cc7)c7ccc8ccccc8c7)cc6)c45)cc3)c3ccc4ccccc4c3)cc2)cc1]

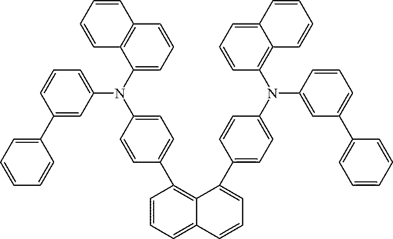 [SMILES: c1ccc(-c2cccc(N(c3ccc(-c4cccc5cccc(-c6ccc(N(c7cccc(-c8ccccc8)c7)c7cccc8ccccc78)cc6)c45)cc3)c3cccc4ccccc34)c2)cc1]

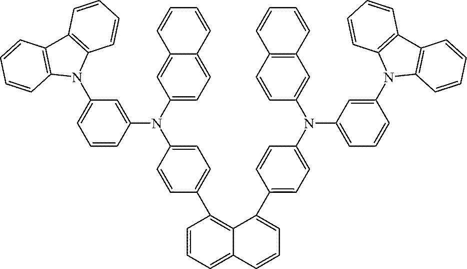 [SMILES: c1cc(N(c2ccc(-c3cccc4cccc(-c5ccc(N(c6cccc(-n7c8ccccc8c8ccccc87)c6)c6ccc7ccccc7c6)cc5)c34)cc2)c2ccc3ccccc3c2)cc(-n2c3ccccc3c3ccccc32)c1]

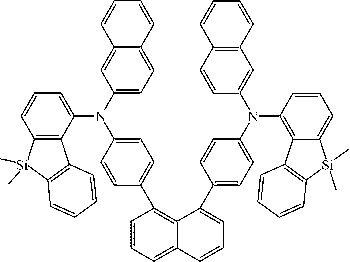

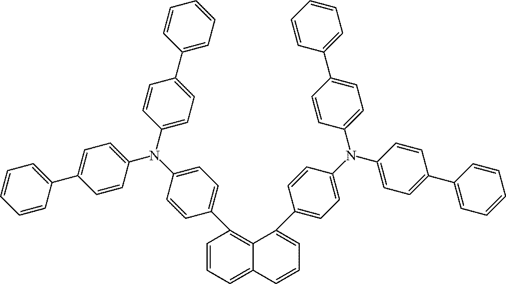 [SMILES: c1ccc(-c2ccc(N(c3ccc(-c4ccccc4)cc3)c3ccc(-c4cccc5cccc(-c6ccc(N(c7ccc(-c8ccccc8)cc7)c7ccc(-c8ccccc8)cc7)cc6)c45)cc3)cc2)cc1]

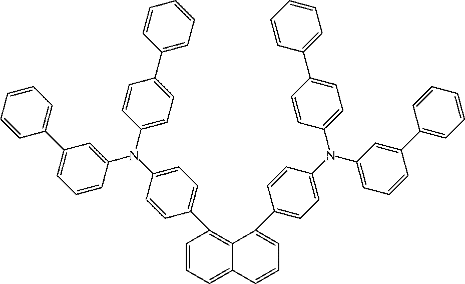 [SMILES: c1ccc(-c2ccc(N(c3ccc(-c4cccc5cccc(-c6ccc(N(c7ccc(-c8ccccc8)cc7)c7cccc(-c8ccccc8)c7)cc6)c45)cc3)c3cccc(-c4ccccc4)c3)cc2)cc1]

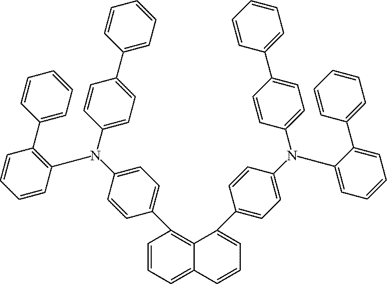 [SMILES: c1ccc(-c2ccc(N(c3ccc(-c4cccc5cccc(-c6ccc(N(c7ccc(-c8ccccc8)cc7)c7ccccc7-c7ccccc7)cc6)c45)cc3)c3ccccc3-c3ccccc3)cc2)cc1]

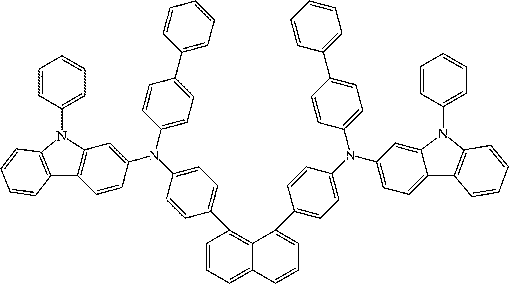 [SMILES: c1ccc(-c2ccc(N(c3ccc(-c4cccc5cccc(-c6ccc(N(c7ccc(-c8ccccc8)cc7)c7ccc8c9ccccc9n(-c9ccccc9)c8c7)cc6)c45)cc3)c3ccc4c5ccccc5n(-c5ccccc5)c4c3)cc2)cc1]

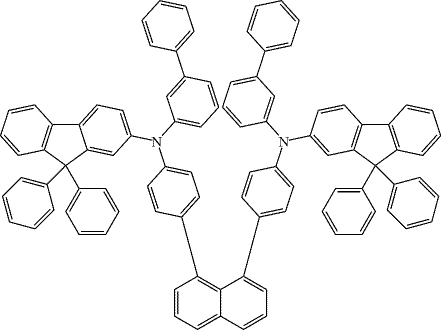 [SMILES: c1ccc(-c2cccc(N(c3ccc(-c4cccc5cccc(-c6ccc(N(c7cccc(-c8ccccc8)c7)c7ccc8c(c7)C(c7ccccc7)(c7ccccc7)c7ccccc7-8)cc6)c45)cc3)c3ccc4c(c3)C(c3ccccc3)(c3ccccc3)c3ccccc3-4)c2)cc1]

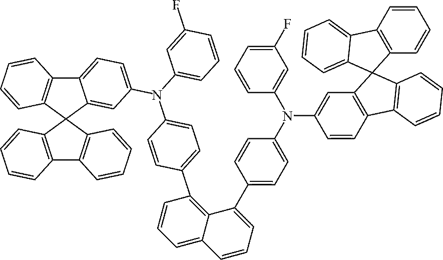 [SMILES: Fc1cccc(N(c2ccc(-c3cccc4cccc(-c5ccc(N(c6cccc(F)c6)c6ccc7c(c6)C6(c8ccccc8-c8ccccc86)c6ccccc6-7)cc5)c34)cc2)c2ccc3c(c2)C2(c4ccccc4-c4ccccc42)c2ccccc2-3)c1]

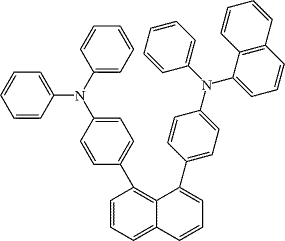 [SMILES: c1ccc(N(c2ccccc2)c2ccc(-c3cccc4cccc(-c5ccc(N(c6ccccc6)c6cccc7ccccc67)cc5)c34)cc2)cc1]

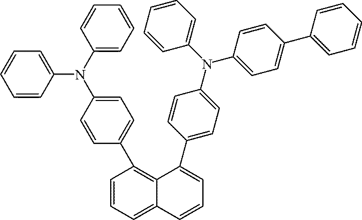 [SMILES: c1ccc(-c2ccc(N(c3ccccc3)c3ccc(-c4cccc5cccc(-c6ccc(N(c7ccccc7)c7ccccc7)cc6)c45)cc3)cc2)cc1]

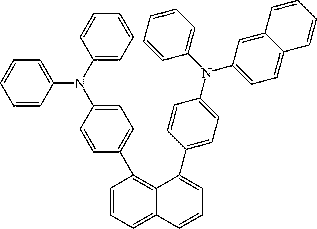 [SMILES: c1ccc(N(c2ccccc2)c2ccc(-c3cccc4cccc(-c5ccc(N(c6ccccc6)c6ccc7ccccc7c6)cc5)c34)cc2)cc1]

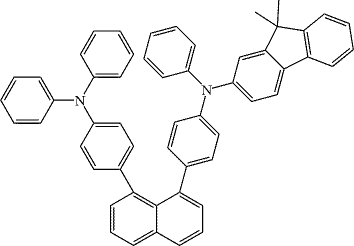 [SMILES: CC1(C)c2ccccc2-c2ccc(N(c3ccccc3)c3ccc(-c4cccc5cccc(-c6ccc(N(c7ccccc7)c7ccccc7)cc6)c45)cc3)cc21]

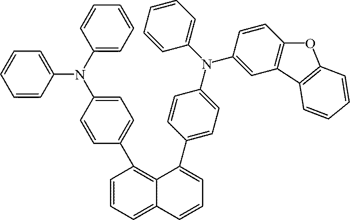 [SMILES: c1ccc(N(c2ccccc2)c2ccc(-c3cccc4cccc(-c5ccc(N(c6ccccc6)c6ccc7oc8ccccc8c7c6)cc5)c34)cc2)cc1]

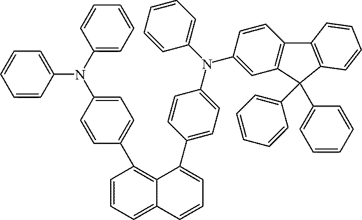 [SMILES: c1ccc(N(c2ccccc2)c2ccc(-c3cccc4cccc(-c5ccc(N(c6ccccc6)c6ccc7c(c6)C(c6ccccc6)(c6ccccc6)c6ccccc6-7)cc5)c34)cc2)cc1]

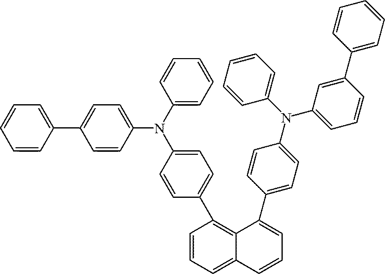 [SMILES: c1ccc(-c2ccc(N(c3ccccc3)c3ccc(-c4cccc5cccc(-c6ccc(N(c7ccccc7)c7cccc(-c8ccccc8)c7)cc6)c45)cc3)cc2)cc1]

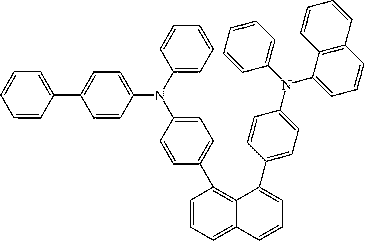 [SMILES: c1ccc(-c2ccc(N(c3ccccc3)c3ccc(-c4cccc5cccc(-c6ccc(N(c7ccccc7)c7cccc8ccccc78)cc6)c45)cc3)cc2)cc1]

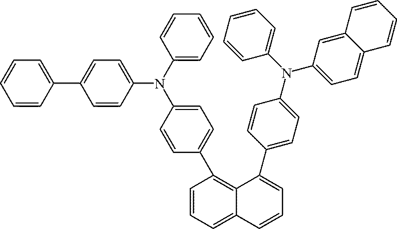 [SMILES: c1ccc(-c2ccc(N(c3ccccc3)c3ccc(-c4cccc5cccc(-c6ccc(N(c7ccccc7)c7ccc8ccccc8c7)cc6)c45)cc3)cc2)cc1]

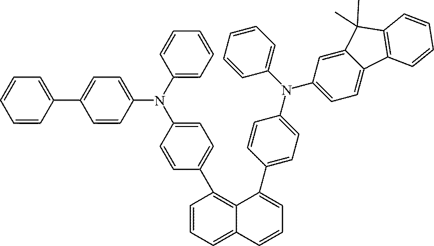 [SMILES: CC1(C)c2ccccc2-c2ccc(N(c3ccccc3)c3ccc(-c4cccc5cccc(-c6ccc(N(c7ccccc7)c7ccc(-c8ccccc8)cc7)cc6)c45)cc3)cc21]

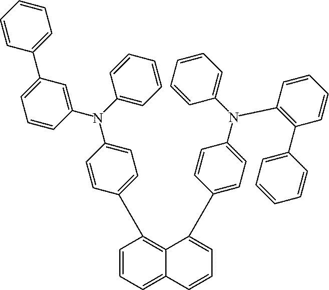 [SMILES: c1ccc(-c2cccc(N(c3ccccc3)c3ccc(-c4cccc5cccc(-c6ccc(N(c7ccccc7)c7ccccc7-c7ccccc7)cc6)c45)cc3)c2)cc1]

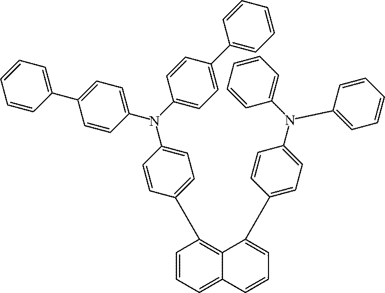 [SMILES: c1ccc(-c2ccc(N(c3ccc(-c4ccccc4)cc3)c3ccc(-c4cccc5cccc(-c6ccc(N(c7ccccc7)c7ccccc7)cc6)c45)cc3)cc2)cc1]

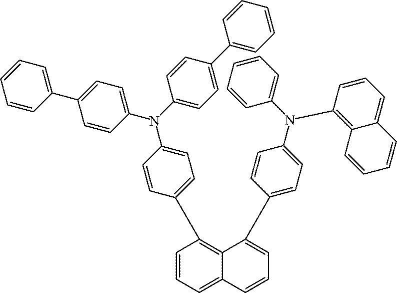 [SMILES: c1ccc(-c2ccc(N(c3ccc(-c4ccccc4)cc3)c3ccc(-c4cccc5cccc(-c6ccc(N(c7ccccc7)c7cccc8ccccc78)cc6)c45)cc3)cc2)cc1]

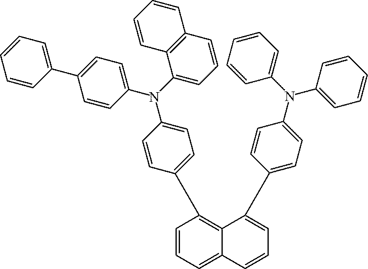 [SMILES: c1ccc(-c2ccc(N(c3ccc(-c4cccc5cccc(-c6ccc(N(c7ccccc7)c7ccccc7)cc6)c45)cc3)c3cccc4ccccc34)cc2)cc1]

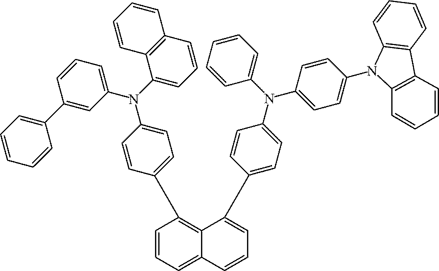 [SMILES: c1ccc(-c2cccc(N(c3ccc(-c4cccc5cccc(-c6ccc(N(c7ccccc7)c7ccc(-n8c9ccccc9c9ccccc98)cc7)cc6)c45)cc3)c3cccc4ccccc34)c2)cc1]

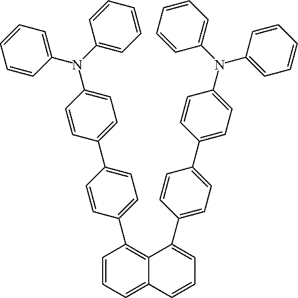 [SMILES: c1ccc(N(c2ccccc2)c2ccc(-c3ccc(-c4cccc5cccc(-c6ccc(-c7ccc(N(c8ccccc8)c8ccccc8)cc7)cc6)c45)cc3)cc2)cc1]

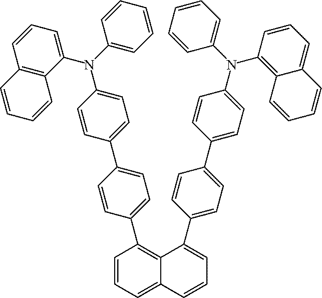 [SMILES: c1ccc(N(c2ccc(-c3ccc(-c4cccc5cccc(-c6ccc(-c7ccc(N(c8ccccc8)c8cccc9ccccc89)cc7)cc6)c45)cc3)cc2)c2cccc3ccccc23)cc1]

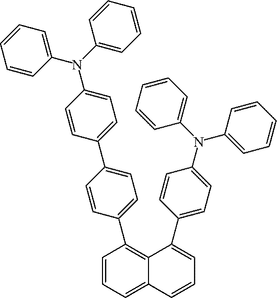 [SMILES: c1ccc(N(c2ccccc2)c2ccc(-c3ccc(-c4cccc5cccc(-c6ccc(N(c7ccccc7)c7ccccc7)cc6)c45)cc3)cc2)cc1]

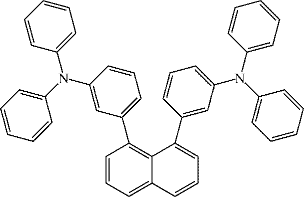 [SMILES: c1ccc(N(c2ccccc2)c2cccc(-c3cccc4cccc(-c5cccc(N(c6ccccc6)c6ccccc6)c5)c34)c2)cc1]

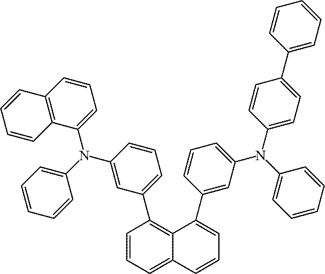 [SMILES: c1ccc(-c2ccc(N(c3ccccc3)c3cccc(-c4cccc5cccc(-c6cccc(N(c7ccccc7)c7cccc8ccccc78)c6)c45)c3)cc2)cc1]

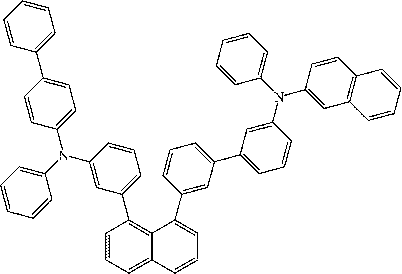 [SMILES: c1ccc(-c2ccc(N(c3ccccc3)c3cccc(-c4cccc5cccc(-c6cccc(-c7cccc(N(c8ccccc8)c8ccc9ccccc9c8)c7)c6)c45)c3)cc2)cc1]

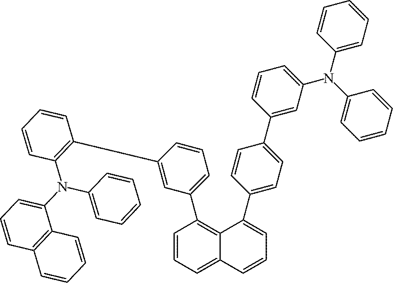 [SMILES: c1ccc(N(c2ccccc2)c2cccc(-c3ccc(-c4cccc5cccc(-c6cccc(-c7ccccc7N(c7ccccc7)c7cccc8ccccc78)c6)c45)cc3)c2)cc1]

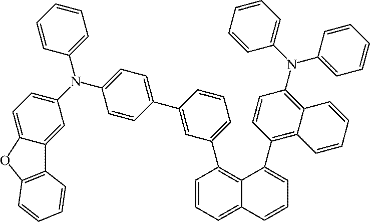 [SMILES: c1ccc(N(c2ccc(-c3cccc(-c4cccc5cccc(-c6ccc(N(c7ccccc7)c7ccccc7)c7ccccc67)c45)c3)cc2)c2ccc3oc4ccccc4c3c2)cc1]

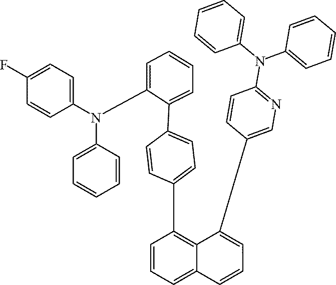 [SMILES: Fc1ccc(N(c2ccccc2)c2ccccc2-c2ccc(-c3cccc4cccc(-c5ccc(N(c6ccccc6)c6ccccc6)nc5)c34)cc2)cc1]

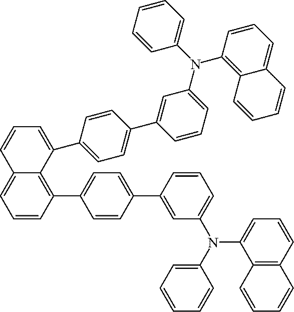 [SMILES: c1ccc(N(c2cccc(-c3ccc(-c4cccc5cccc(-c6ccc(-c7cccc(N(c8ccccc8)c8cccc9ccccc89)c7)cc6)c45)cc3)c2)c2cccc3ccccc23)cc1]

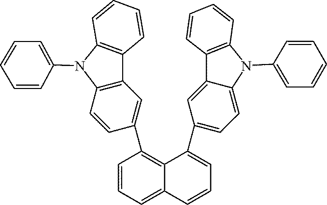 [SMILES: c1ccc(-n2c3ccccc3c3cc(-c4cccc5cccc(-c6ccc7c(c6)c6ccccc6n7-c6ccccc6)c45)ccc32)cc1]

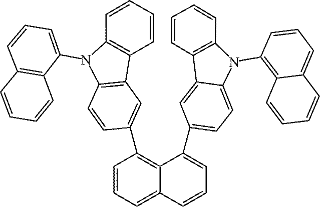 [SMILES: c1ccc2c(-n3c4ccccc4c4cc(-c5cccc6cccc(-c7ccc8c(c7)c7ccccc7n8-c7cccc8ccccc78)c56)ccc43)cccc2c1]

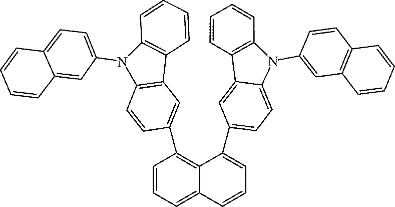 [SMILES: c1ccc2cc(-n3c4ccccc4c4cc(-c5cccc6cccc(-c7ccc8c(c7)c7ccccc7n8-c7ccc8ccccc8c7)c56)ccc43)ccc2c1]

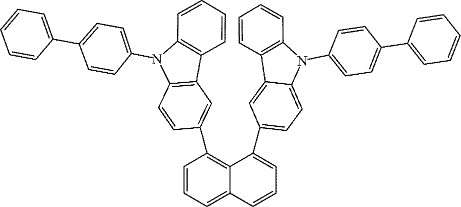 [SMILES: c1ccc(-c2ccc(-n3c4ccccc4c4cc(-c5cccc6cccc(-c7ccc8c(c7)c7ccccc7n8-c7ccc(-c8ccccc8)cc7)c56)ccc43)cc2)cc1]

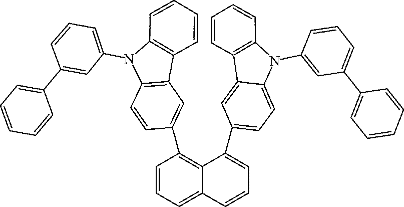 [SMILES: c1ccc(-c2cccc(-n3c4ccccc4c4cc(-c5cccc6cccc(-c7ccc8c(c7)c7ccccc7n8-c7cccc(-c8ccccc8)c7)c56)ccc43)c2)cc1]

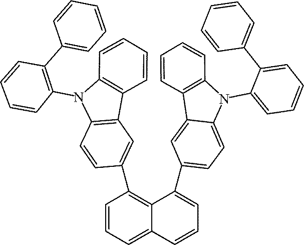 [SMILES: c1ccc(-c2ccccc2-n2c3ccccc3c3cc(-c4cccc5cccc(-c6ccc7c(c6)c6ccccc6n7-c6ccccc6-c6ccccc6)c45)ccc32)cc1]

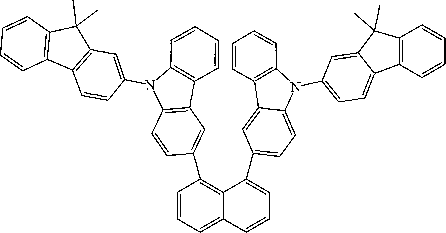 [SMILES: CC1(C)c2ccccc2-c2ccc(-n3c4ccccc4c4cc(-c5cccc6cccc(-c7ccc8c(c7)c7ccccc7n8-c7ccc8c(c7)C(C)(C)c7ccccc7-8)c56)ccc43)cc21]

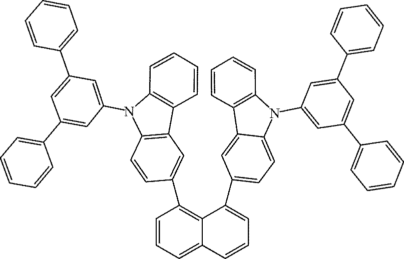 [SMILES: c1ccc(-c2cc(-c3ccccc3)cc(-n3c4ccccc4c4cc(-c5cccc6cccc(-c7ccc8c(c7)c7ccccc7n8-c7cc(-c8ccccc8)cc(-c8ccccc8)c7)c56)ccc43)c2)cc1]

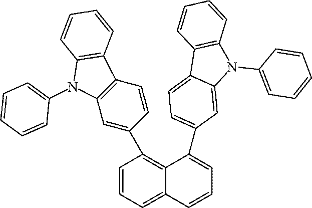 [SMILES: c1ccc(-n2c3ccccc3c3ccc(-c4cccc5cccc(-c6ccc7c8ccccc8n(-c8ccccc8)c7c6)c45)cc32)cc1]

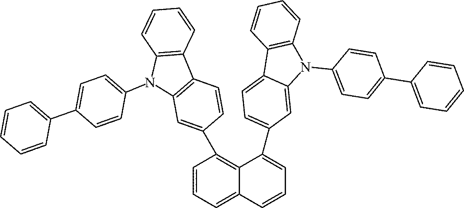 [SMILES: c1ccc(-c2ccc(-n3c4ccccc4c4ccc(-c5cccc6cccc(-c7ccc8c9ccccc9n(-c9ccc(-c%10ccccc%10)cc9)c8c7)c56)cc43)cc2)cc1]

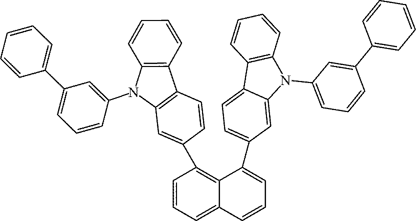 [SMILES: c1ccc(-c2cccc(-n3c4ccccc4c4ccc(-c5cccc6cccc(-c7ccc8c9ccccc9n(-c9cccc(-c%10ccccc%10)c9)c8c7)c56)cc43)c2)cc1]

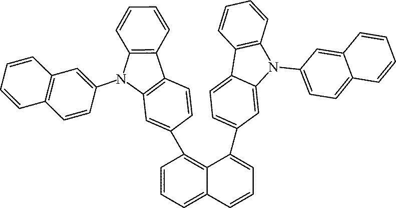 [SMILES: c1ccc2cc(-n3c4ccccc4c4ccc(-c5cccc6cccc(-c7ccc8c9ccccc9n(-c9ccc%10ccccc%10c9)c8c7)c56)cc43)ccc2c1]

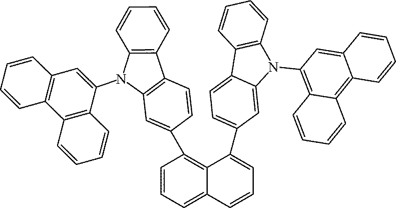 [SMILES: c1cc(-c2ccc3c4ccccc4n(-c4cc5ccccc5c5ccccc45)c3c2)c2c(-c3ccc4c5ccccc5n(-c5cc6ccccc6c6ccccc56)c4c3)cccc2c1]

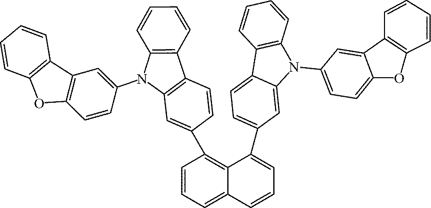 [SMILES: c1cc(-c2ccc3c4ccccc4n(-c4ccc5oc6ccccc6c5c4)c3c2)c2c(-c3ccc4c5ccccc5n(-c5ccc6oc7ccccc7c6c5)c4c3)cccc2c1]

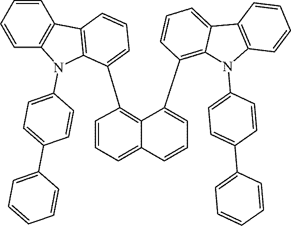 [SMILES: c1ccc(-c2ccc(-n3c4ccccc4c4cccc(-c5cccc6cccc(-c7cccc8c9ccccc9n(-c9ccc(-c%10ccccc%10)cc9)c78)c56)c43)cc2)cc1]

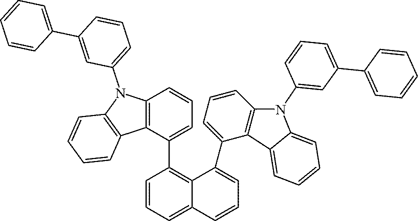 [SMILES: c1ccc(-c2cccc(-n3c4ccccc4c4c(-c5cccc6cccc(-c7cccc8c7c7ccccc7n8-c7cccc(-c8ccccc8)c7)c56)cccc43)c2)cc1]

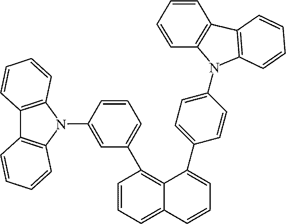 [SMILES: c1cc(-c2cccc3cccc(-c4ccc(-n5c6ccccc6c6ccccc65)cc4)c23)cc(-n2c3ccccc3c3ccccc32)c1]

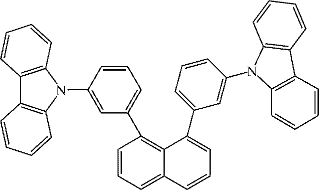 [SMILES: c1cc(-c2cccc3cccc(-c4cccc(-n5c6ccccc6c6ccccc65)c4)c23)cc(-n2c3ccccc3c3ccccc32)c1]

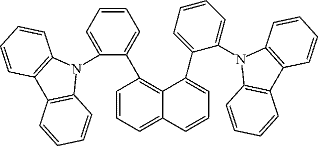 [SMILES: c1ccc(-n2c3ccccc3c3ccccc32)c(-c2cccc3cccc(-c4ccccc4-n4c5ccccc5c5ccccc54)c23)c1]

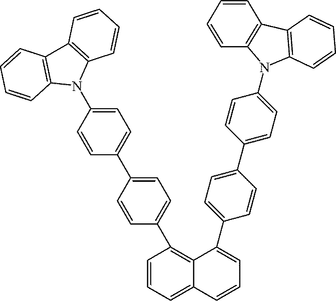 [SMILES: c1cc(-c2ccc(-c3ccc(-n4c5ccccc5c5ccccc54)cc3)cc2)c2c(-c3ccc(-c4ccc(-n5c6ccccc6c6ccccc65)cc4)cc3)cccc2c1]

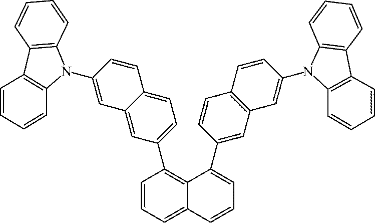 [SMILES: c1cc(-c2ccc3ccc(-n4c5ccccc5c5ccccc54)cc3c2)c2c(-c3ccc4ccc(-n5c6ccccc6c6ccccc65)cc4c3)cccc2c1]

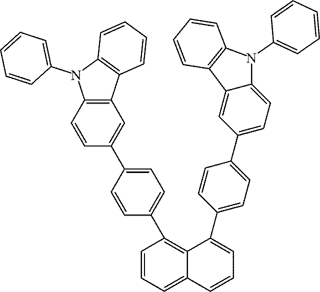 [SMILES: c1ccc(-n2c3ccccc3c3cc(-c4ccc(-c5cccc6cccc(-c7ccc(-c8ccc9c(c8)c8ccccc8n9-c8ccccc8)cc7)c56)cc4)ccc32)cc1]

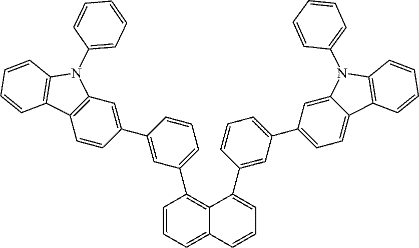 [SMILES: c1ccc(-n2c3ccccc3c3ccc(-c4cccc(-c5cccc6cccc(-c7cccc(-c8ccc9c%10ccccc%10n(-c%10ccccc%10)c9c8)c7)c56)c4)cc32)cc1]

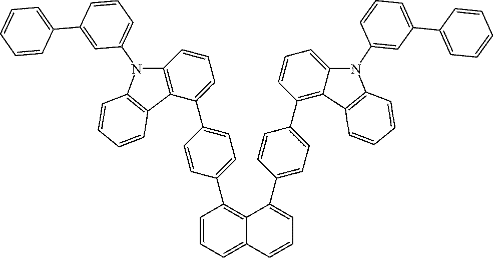 [SMILES: c1ccc(-c2cccc(-n3c4ccccc4c4c(-c5ccc(-c6cccc7cccc(-c8ccc(-c9cccc%10c9c9ccccc9n%10-c9cccc(-c%10ccccc%10)c9)cc8)c67)cc5)cccc43)c2)cc1]

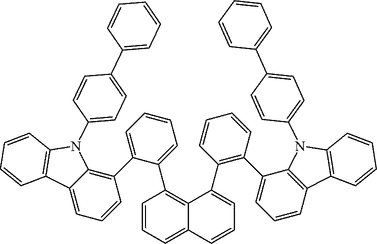 [SMILES: c1ccc(-c2ccc(-n3c4ccccc4c4cccc(-c5ccccc5-c5cccc6cccc(-c7ccccc7-c7cccc8c9ccccc9n(-c9ccc(-c%10ccccc%10)cc9)c78)c56)c43)cc2)cc1]

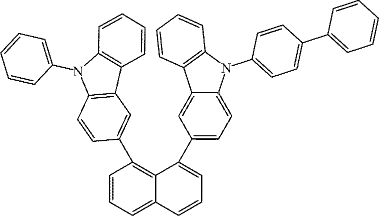 [SMILES: c1ccc(-c2ccc(-n3c4ccccc4c4cc(-c5cccc6cccc(-c7ccc8c(c7)c7ccccc7n8-c7ccccc7)c56)ccc43)cc2)cc1]

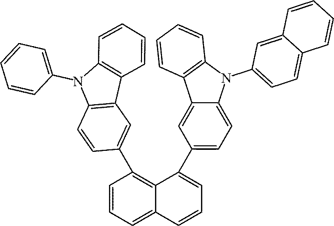 [SMILES: c1ccc(-n2c3ccccc3c3cc(-c4cccc5cccc(-c6ccc7c(c6)c6ccccc6n7-c6ccc7ccccc7c6)c45)ccc32)cc1]

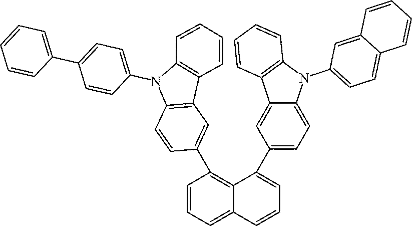 [SMILES: c1ccc(-c2ccc(-n3c4ccccc4c4cc(-c5cccc6cccc(-c7ccc8c(c7)c7ccccc7n8-c7ccc8ccccc8c7)c56)ccc43)cc2)cc1]

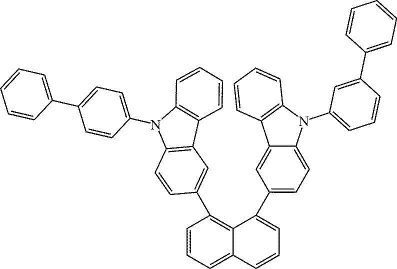 [SMILES: c1ccc(-c2ccc(-n3c4ccccc4c4cc(-c5cccc6cccc(-c7ccc8c(c7)c7ccccc7n8-c7cccc(-c8ccccc8)c7)c56)ccc43)cc2)cc1]

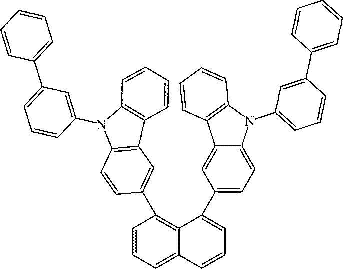 [SMILES: c1ccc(-c2cccc(-n3c4ccccc4c4cc(-c5cccc6cccc(-c7ccc8c(c7)c7ccccc7n8-c7cccc(-c8ccccc8)c7)c56)ccc43)c2)cc1]

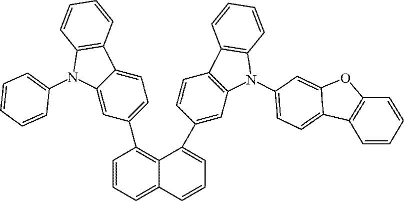 [SMILES: c1ccc(-n2c3ccccc3c3ccc(-c4cccc5cccc(-c6ccc7c8ccccc8n(-c8ccc9c(c8)oc8ccccc89)c7c6)c45)cc32)cc1]

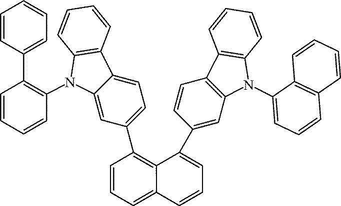 [SMILES: c1ccc(-c2ccccc2-n2c3ccccc3c3ccc(-c4cccc5cccc(-c6ccc7c8ccccc8n(-c8cccc9ccccc89)c7c6)c45)cc32)cc1]

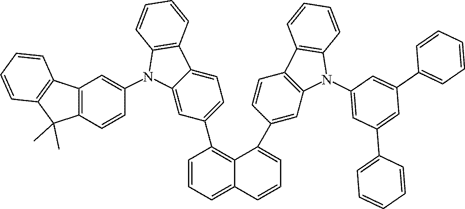 [SMILES: CC1(C)c2ccccc2-c2cc(-n3c4ccccc4c4ccc(-c5cccc6cccc(-c7ccc8c9ccccc9n(-c9cc(-c%10ccccc%10)cc(-c%10ccccc%10)c9)c8c7)c56)cc43)ccc21]

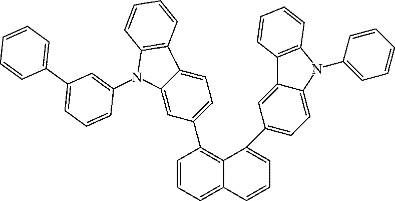 [SMILES: c1ccc(-c2cccc(-n3c4ccccc4c4ccc(-c5cccc6cccc(-c7ccc8c(c7)c7ccccc7n8-c7ccccc7)c56)cc43)c2)cc1]

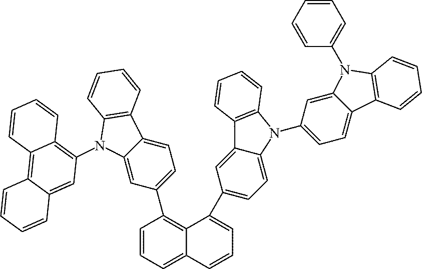 [SMILES: c1ccc(-n2c3ccccc3c3ccc(-n4c5ccccc5c5cc(-c6cccc7cccc(-c8ccc9c%10ccccc%10n(-c%10cc%11ccccc%11c%11ccccc%10%11)c9c8)c67)ccc54)cc32)cc1]

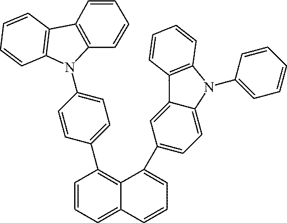 [SMILES: c1ccc(-n2c3ccccc3c3cc(-c4cccc5cccc(-c6ccc(-n7c8ccccc8c8ccccc87)cc6)c45)ccc32)cc1]

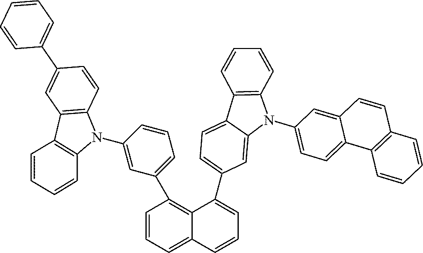 [SMILES: c1ccc(-c2ccc3c(c2)c2ccccc2n3-c2cccc(-c3cccc4cccc(-c5ccc6c7ccccc7n(-c7ccc8c(ccc9ccccc98)c7)c6c5)c34)c2)cc1]

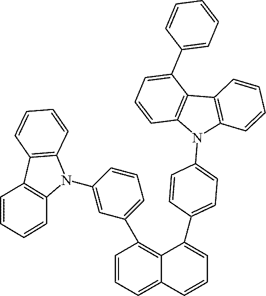 [SMILES: c1ccc(-c2cccc3c2c2ccccc2n3-c2ccc(-c3cccc4cccc(-c5cccc(-n6c7ccccc7c7ccccc76)c5)c34)cc2)cc1]

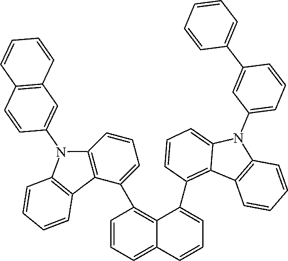 [SMILES: c1ccc(-c2cccc(-n3c4ccccc4c4c(-c5cccc6cccc(-c7cccc8c7c7ccccc7n8-c7ccc8ccccc8c7)c56)cccc43)c2)cc1]

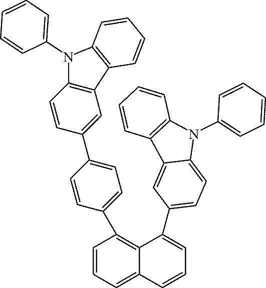 [SMILES: c1ccc(-n2c3ccccc3c3cc(-c4ccc(-c5cccc6cccc(-c7ccc8c(c7)c7ccccc7n8-c7ccccc7)c56)cc4)ccc32)cc1]

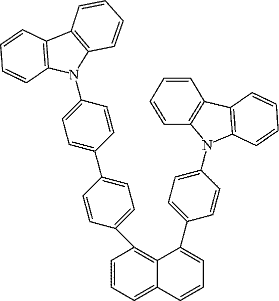 [SMILES: c1cc(-c2ccc(-c3ccc(-n4c5ccccc5c5ccccc54)cc3)cc2)c2c(-c3ccc(-n4c5ccccc5c5ccccc54)cc3)cccc2c1]

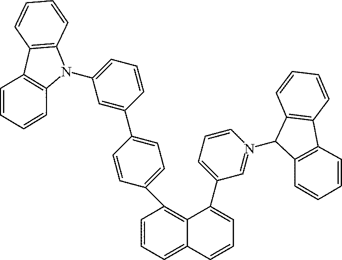

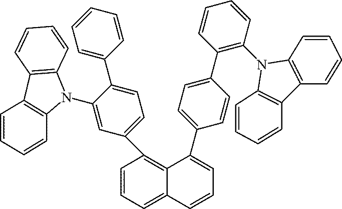 [SMILES: c1ccc(-c2ccc(-c3cccc4cccc(-c5ccc(-c6ccccc6-n6c7ccccc7c7ccccc76)cc5)c34)cc2-n2c3ccccc3c3ccccc32)cc1]

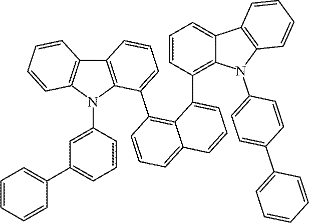 [SMILES: c1ccc(-c2ccc(-n3c4ccccc4c4cccc(-c5cccc6cccc(-c7cccc8c9ccccc9n(-c9cccc(-c%10ccccc%10)c9)c78)c56)c43)cc2)cc1]

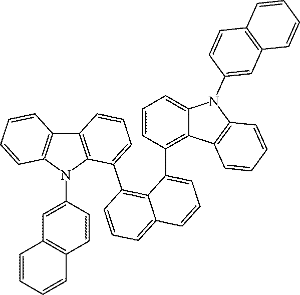 [SMILES: c1ccc2cc(-n3c4ccccc4c4c(-c5cccc6cccc(-c7cccc8c9ccccc9n(-c9ccc%10ccccc%10c9)c78)c56)cccc43)ccc2c1]

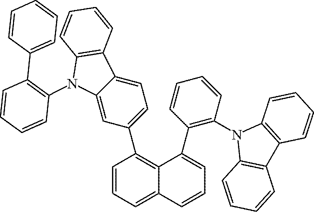 [SMILES: c1ccc(-c2ccccc2-n2c3ccccc3c3ccc(-c4cccc5cccc(-c6ccccc6-n6c7ccccc7c7ccccc76)c45)cc32)cc1]

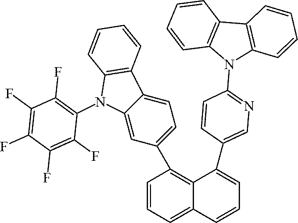 [SMILES: Fc1c(F)c(F)c(-n2c3ccccc3c3ccc(-c4cccc5cccc(-c6ccc(-n7c8ccccc8c8ccccc87)nc6)c45)cc32)c(F)c1F]

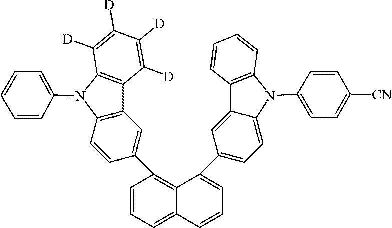 [SMILES: [2H]c1c([2H])c([2H])c2c(c1[2H])c1cc(-c3cccc4cccc(-c5ccc6c(c5)c5ccccc5n6-c5ccc(C#N)cc5)c34)ccc1n2-c1ccccc1]

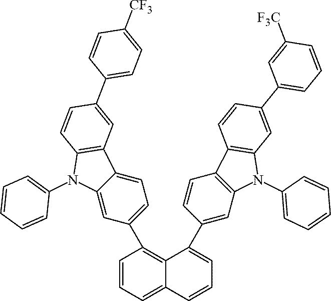 [SMILES: FC(F)(F)c1ccc(-c2ccc3c(c2)c2ccc(-c4cccc5cccc(-c6ccc7c8ccc(-c9cccc(C(F)(F)F)c9)cc8n(-c8ccccc8)c7c6)c45)cc2n3-c2ccccc2)cc1]

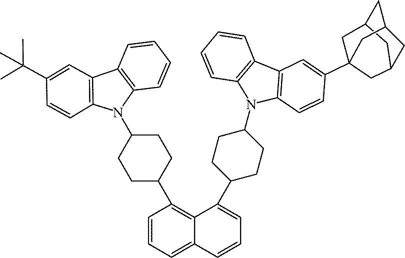 [SMILES: CC(C)(C)c1ccc2c(c1)c1ccccc1n2C1CCC(c2cccc3cccc(C4CCC(n5c6ccccc6c6cc(C78CC9CC(CC(C9)C7)C8)ccc65)CC4)c23)CC1]

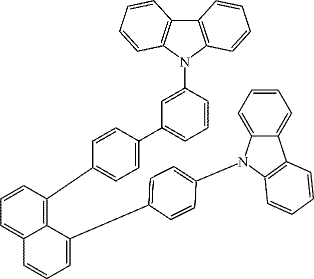 [SMILES: c1cc(-c2ccc(-c3cccc4cccc(-c5ccc(-n6c7ccccc7c7ccccc76)cc5)c34)cc2)cc(-n2c3ccccc3c3ccccc32)c1]

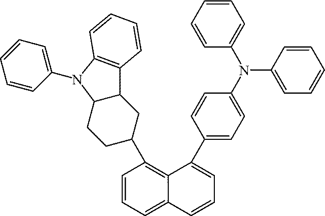 [SMILES: c1ccc(N(c2ccccc2)c2ccc(-c3cccc4cccc(C5CCC6C(C5)c5ccccc5N6c5ccccc5)c34)cc2)cc1]

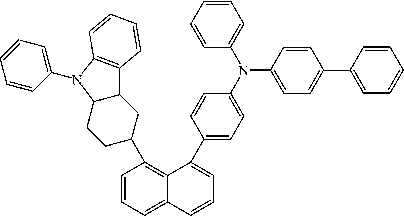 [SMILES: c1ccc(-c2ccc(N(c3ccccc3)c3ccc(-c4cccc5cccc(C6CCC7C(C6)c6ccccc6N7c6ccccc6)c45)cc3)cc2)cc1]

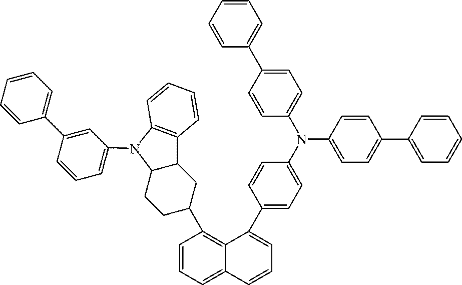 [SMILES: c1ccc(-c2ccc(N(c3ccc(-c4ccccc4)cc3)c3ccc(-c4cccc5cccc(C6CCC7C(C6)c6ccccc6N7c6cccc(-c7ccccc7)c6)c45)cc3)cc2)cc1]

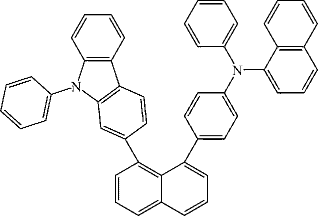 [SMILES: c1ccc(N(c2ccc(-c3cccc4cccc(-c5ccc6c7ccccc7n(-c7ccccc7)c6c5)c34)cc2)c2cccc3ccccc23)cc1]

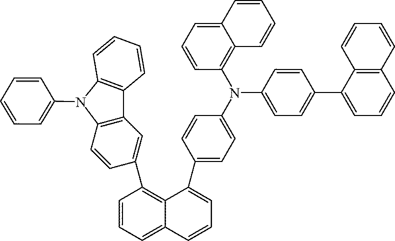 [SMILES: c1ccc(-n2c3ccccc3c3cc(-c4cccc5cccc(-c6ccc(N(c7ccc(-c8cccc9ccccc89)cc7)c7cccc8ccccc78)cc6)c45)ccc32)cc1]

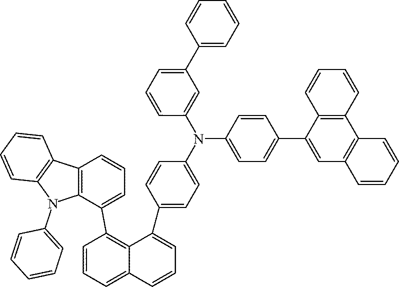 [SMILES: c1ccc(-c2cccc(N(c3ccc(-c4cc5ccccc5c5ccccc45)cc3)c3ccc(-c4cccc5cccc(-c6cccc7c8ccccc8n(-c8ccccc8)c67)c45)cc3)c2)cc1]

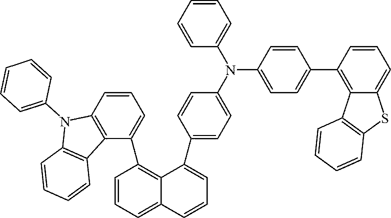 [SMILES: c1ccc(N(c2ccc(-c3cccc4cccc(-c5cccc6c5c5ccccc5n6-c5ccccc5)c34)cc2)c2ccc(-c3cccc4sc5ccccc5c34)cc2)cc1]

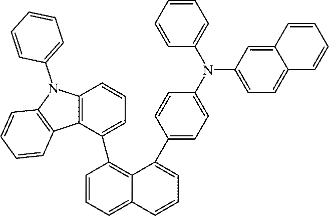 [SMILES: c1ccc(N(c2ccc(-c3cccc4cccc(-c5cccc6c5c5ccccc5n6-c5ccccc5)c34)cc2)c2ccc3ccccc3c2)cc1]

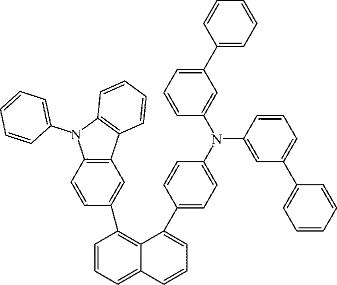 [SMILES: c1ccc(-c2cccc(N(c3ccc(-c4cccc5cccc(-c6ccc7c(c6)c6ccccc6n7-c6ccccc6)c45)cc3)c3cccc(-c4ccccc4)c3)c2)cc1]

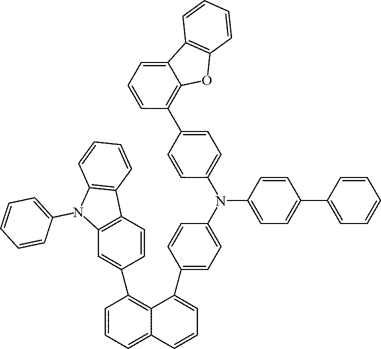 [SMILES: c1ccc(-c2ccc(N(c3ccc(-c4cccc5c4oc4ccccc45)cc3)c3ccc(-c4cccc5cccc(-c6ccc7c8ccccc8n(-c8ccccc8)c7c6)c45)cc3)cc2)cc1]

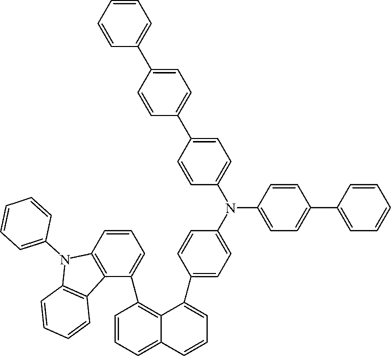 [SMILES: c1ccc(-c2ccc(-c3ccc(N(c4ccc(-c5ccccc5)cc4)c4ccc(-c5cccc6cccc(-c7cccc8c7c7ccccc7n8-c7ccccc7)c56)cc4)cc3)cc2)cc1]

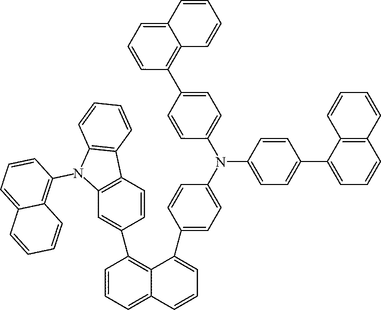 [SMILES: c1ccc2c(-c3ccc(N(c4ccc(-c5cccc6ccccc56)cc4)c4ccc(-c5cccc6cccc(-c7ccc8c9ccccc9n(-c9cccc%10ccccc9%10)c8c7)c56)cc4)cc3)cccc2c1]

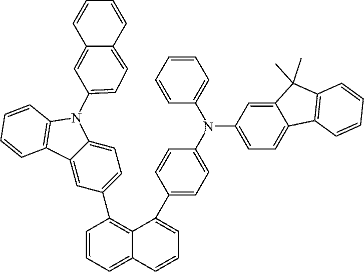 [SMILES: CC1(C)c2ccccc2-c2ccc(N(c3ccccc3)c3ccc(-c4cccc5cccc(-c6ccc7c(c6)c6ccccc6n7-c6ccc7ccccc7c6)c45)cc3)cc21]

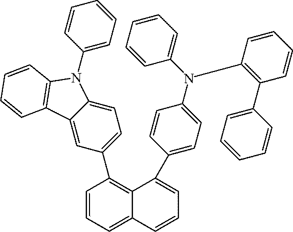 [SMILES: c1ccc(-c2ccccc2N(c2ccccc2)c2ccc(-c3cccc4cccc(-c5ccc6c(c5)c5ccccc5n6-c5ccccc5)c34)cc2)cc1]

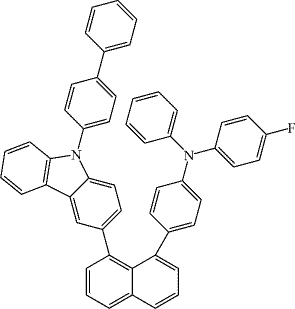 [SMILES: Fc1ccc(N(c2ccccc2)c2ccc(-c3cccc4cccc(-c5ccc6c(c5)c5ccccc5n6-c5ccc(-c6ccccc6)cc5)c34)cc2)cc1]

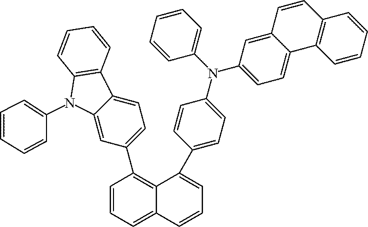 [SMILES: c1ccc(N(c2ccc(-c3cccc4cccc(-c5ccc6c7ccccc7n(-c7ccccc7)c6c5)c34)cc2)c2ccc3c(ccc4ccccc43)c2)cc1]

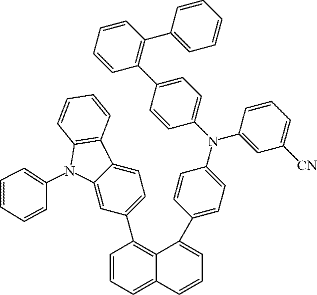 [SMILES: N#Cc1cccc(N(c2ccc(-c3ccccc3-c3ccccc3)cc2)c2ccc(-c3cccc4cccc(-c5ccc6c7ccccc7n(-c7ccccc7)c6c5)c34)cc2)c1]

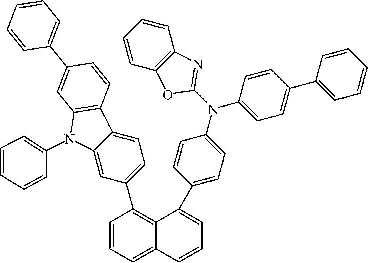 [SMILES: c1ccc(-c2ccc(N(c3ccc(-c4cccc5cccc(-c6ccc7c8ccc(-c9ccccc9)cc8n(-c8ccccc8)c7c6)c45)cc3)c3nc4ccccc4o3)cc2)cc1]

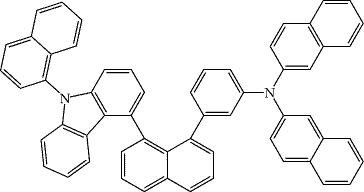 [SMILES: c1cc(-c2cccc3cccc(-c4cccc5c4c4ccccc4n5-c4cccc5ccccc45)c23)cc(N(c2ccc3ccccc3c2)c2ccc3ccccc3c2)c1]

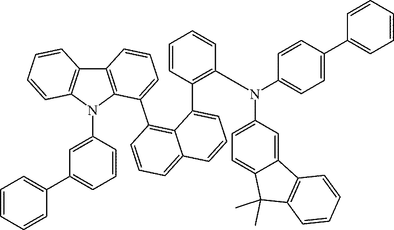 [SMILES: CC1(C)c2ccccc2-c2cc(N(c3ccc(-c4ccccc4)cc3)c3ccccc3-c3cccc4cccc(-c5cccc6c7ccccc7n(-c7cccc(-c8ccccc8)c7)c56)c34)ccc21]

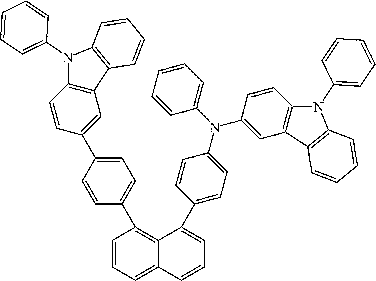 [SMILES: c1ccc(N(c2ccc(-c3cccc4cccc(-c5ccc(-c6ccc7c(c6)c6ccccc6n7-c6ccccc6)cc5)c34)cc2)c2ccc3c(c2)c2ccccc2n3-c2ccccc2)cc1]

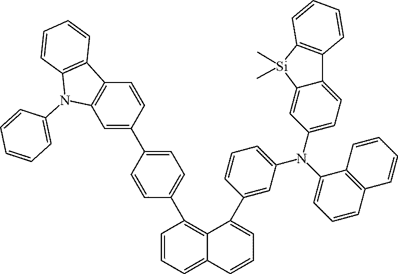 [SMILES: C[Si]1(C)c2ccccc2-c2ccc(N(c3cccc(-c4cccc5cccc(-c6ccc(-c7ccc8c9ccccc9n(-c9ccccc9)c8c7)cc6)c45)c3)c3cccc4ccccc34)cc21]

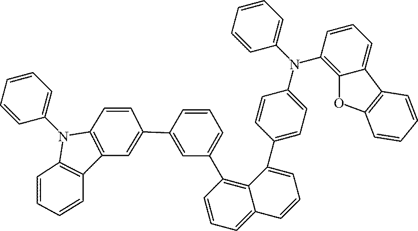 [SMILES: c1ccc(N(c2ccc(-c3cccc4cccc(-c5cccc(-c6ccc7c(c6)c6ccccc6n7-c6ccccc6)c5)c34)cc2)c2cccc3c2oc2ccccc23)cc1]

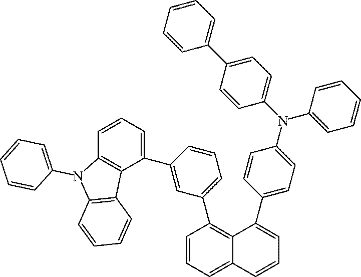 [SMILES: c1ccc(-c2ccc(N(c3ccccc3)c3ccc(-c4cccc5cccc(-c6cccc(-c7cccc8c7c7ccccc7n8-c7ccccc7)c6)c45)cc3)cc2)cc1]

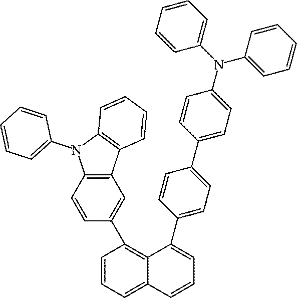 [SMILES: c1ccc(N(c2ccccc2)c2ccc(-c3ccc(-c4cccc5cccc(-c6ccc7c(c6)c6ccccc6n7-c6ccccc6)c45)cc3)cc2)cc1]

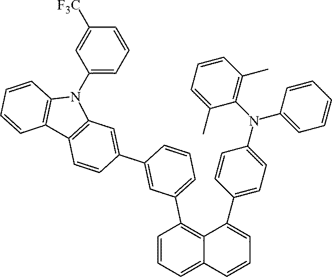 [SMILES: Cc1cccc(C)c1N(c1ccccc1)c1ccc(-c2cccc3cccc(-c4cccc(-c5ccc6c7ccccc7n(-c7cccc(C(F)(F)F)c7)c6c5)c4)c23)cc1]

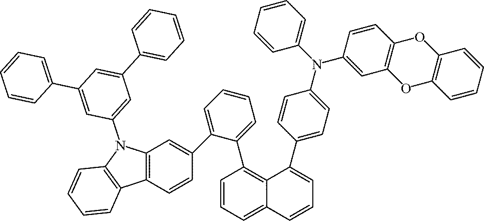 [SMILES: c1ccc(-c2cc(-c3ccccc3)cc(-n3c4ccccc4c4ccc(-c5ccccc5-c5cccc6cccc(-c7ccc(N(c8ccccc8)c8ccc9c(c8)Oc8ccccc8O9)cc7)c56)cc43)c2)cc1]

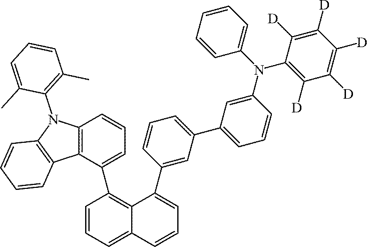 [SMILES: [2H]c1c([2H])c([2H])c(N(c2ccccc2)c2cccc(-c3cccc(-c4cccc5cccc(-c6cccc7c6c6ccccc6n7-c6c(C)cccc6C)c45)c3)c2)c([2H])c1[2H]]

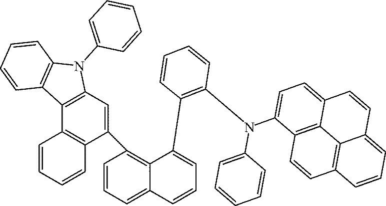 [SMILES: c1ccc(N(c2ccccc2-c2cccc3cccc(-c4cc5c(c6ccccc46)c4ccccc4n5-c4ccccc4)c23)c2ccc3ccc4cccc5ccc2c3c45)cc1]

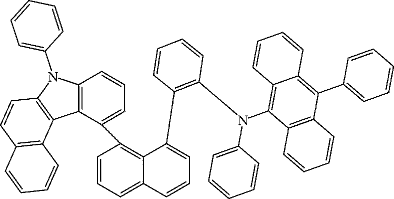 [SMILES: c1ccc(-c2c3ccccc3c(N(c3ccccc3)c3ccccc3-c3cccc4cccc(-c5cccc6c5c5c7ccccc7ccc5n6-c5ccccc5)c34)c3ccccc23)cc1]

A synthesis method of the nitrogen-containing compound provided is not particularly limited in the present disclosure, and those skilled in the art can determine a suitable synthesis method according to the nitrogen-containing compound of the present disclosure in combination with preparation methods provided in synthesis examples. In other words, the synthesis examples of the present disclosure exemplarily provide preparation methods for the nitrogen-containing compounds, and raw materials used may be commercially obtained or obtained by a method well known in the art. Those skilled in the art can obtain all nitrogen-containing compounds provided by the present disclosure according to these exemplary preparation methods, and all specific preparation methods for the nitrogen-containing compounds are not described in detail here, and those skilled in the art should not understand as limiting the present disclosure.

The present disclosure also provides an electronic element, including an anode and a cathode which are oppositely disposed, and a functional layer disposed between the anode and the cathode; the functional layer includes the nitrogen-containing compound of the present disclosure. Generally, the electronic element may be an organic electroluminescent device or a photoelectric conversion device.

Optionally, the functional layer includes a hole transporting layer, and the hole transporting layer may be one layer or two or more layers.

Optionally, the functional layer includes a luminescence layer including a host material and a guest material.

In one embodiment, the hole transporting layer may contain the nitrogen-containing compound. In another embodiment, the host material of the luminescence layer contains the nitrogen-containing compound. The nitrogen-containing compound provided by the present disclosure can be applied to a hole transporting layer of an organic electroluminescent device or as the host material of the luminescence layer, and the luminescence efficiency and service life of the organic electroluminescent device can be increased while guaranteeing a lower driving voltage.

In the present disclosure, the organic electroluminescent device can be a red-light device, a blue-light device or a green-light device.

According to one embodiment of the present disclosure, the electronic element is the organic electroluminescent device. As shown in FIG. 1, the organic electroluminescent device may include an anode 100, a hole transporting layer 320, a luminescence layer 330 as an energy conversion layer, an electron transporting layer 340, and a cathode 200 which are stacked in sequence, and the hole transporting layer 320 includes a first hole transporting layer 321 and a second hole transporting layer 322. As shown in the figure, the first hole transporting layer 321 is closer to the anode 100 than the second hole transporting layer 322.

In the present disclosure, the anode 100 includes an anode material, which is preferably a material with a large work function that facilitates hole injection into the functional layer. Specific examples of the anode material include, but are not limited to, metals such as nickel, platinum, vanadium, chromium, copper, zinc, and gold, or their alloys; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline. A transparent electrode containing indium tin oxide (ITO) as the anode is preferably included.

In the present disclosure, the hole transporting layer 320 may include one or more hole transport materials, and the hole transport materials may be selected from a carbazole polymer, carbazole-linked triarylamine compounds, or other types of compounds. In one embodiment of the present disclosure, the nitrogen-containing compound provided by the present disclosure may be applied to the first hole transporting layer 321 and/or the second hole transporting layer 322 of the organic electroluminescent device. For example, one of the first hole transporting layer 321 and the second hole transporting layer 322 includes the compound of the present disclosure and the other may be composed of a compound HT-02 or HT-01.

In the present disclosure, the luminescence layer 330 may be composed of a single luminescence material and may also include a host material and a guest material. In one specific embodiment, the luminescence layer 330 is composed of the host material and the guest material, and holes injected into the luminescence layer 330 and electrons injected into the luminescence layer 330 can be recombined in the luminescence layer 330 to form excitons, the excitons transfer energy to the host material, and the host material transfers energy to the guest material, thus enabling the guest material to emit light.

The host material of the luminescence layer 330 may be a metal chelate compound, a bis-styryl derivative, an aromatic amine derivative, a dibenzofuran derivative, or other types of materials. The guest material of the luminescence layer 330 may be a compound with a condensed aryl ring or its derivative, a compound with a heteroaryl ring or its derivative, an aromatic amine derivative, or other materials. In one embodiment of the present disclosure, the host material of the luminescence layer 330 includes BH-01 and the guest material includes BD-01. In another embodiment of the present disclosure, the host material of the luminescence layer 330 includes the compound of the present disclosure. For example, the host material of the luminescence layer 330 includes the nitrogen-containing compound of the present disclosure and GH-n1, and the guest material includes Ir(ppy)₃.

The electron transporting layer 340 may be of a single-layer structure or a multi-layer structure and may include one or more electron transport materials, and the electron transport materials can be selected from, but are not limited to, a benzimidazole derivative, an oxadiazole derivative, a quinoxaline derivative, or other electron transport materials. In one embodiment of the present disclosure, the electron transporting layer 340 may be composed of ET-01 and LiQ.

In the present disclosure, specific structures of HT-02, HT-01, BH-01, BD-01, ET-01, GH-n1, and the like are described in Examples below, which will not be repeated here.

In the present disclosure, the cathode 200 may include a cathode material, which is a material with a small work function that facilitates electron injection into the functional layer. Specific embodiments of the cathode material include, but are not limited to, metals such as magnesium, calcium, sodium, potassium, titanium, indium, yttrium, lithium, gadolinium, aluminum, silver, tin, and lead or their alloys; or multilayer materials such as LiF/Al, Liq/Al, LiO₂/Al, LiF/Ca, LiF/Al, and BaF₂/Ca. A metal electrode containing magnesium and silver as the cathode is preferably included.

Optionally, as shown in FIG. 1, a hole injection layer 310 may also be arranged between the anode 100 and the first hole transporting layer 321 to enhance the ability to inject holes into the hole transporting layer. The hole injection layer 310 can be made of a benzidine derivative, a starburst arylamine compound, a phthalocyanine derivative or other materials, which is not specially limited in the present disclosure. For example, the hole injection layer 310 may be composed of F4-TCNQ.

Optionally, as shown in FIG. 1, an electron injection layer 350 may also be arranged between the cathode 200 and the electron transporting layer 340 to enhance the ability to inject electrons into the electron transporting layer 340. The electron injection layer 350 may include an inorganic material such as an alkali metal sulfide, an alkali metal halide and Yb, or may include a complex of an alkali metal and an organic substance. For example, the electron injection layer 350 may include LiQ.

Optionally, as shown in FIG. 1, the hole injection layer 310, the first hole transporting layer 321, the second hole transporting layer 322, the luminescence layer 330, the electron transporting layer 340, and the electron injection layer 350 constitute the functional layer 300.

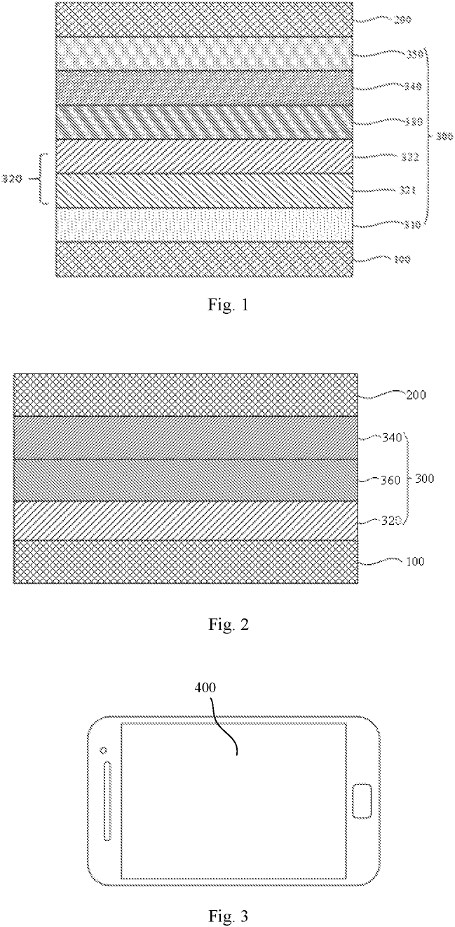

According to another embodiment, the electronic element may be the photoelectric conversion device. As shown in FIG. 2, the photoelectric conversion device may include an anode 100 and a cathode 200 which are disposed oppositely, and a functional layer 300 disposed between the anode 100 and the cathode 200; and the functional layer 300 includes the nitrogen-containing compound provided by the present disclosure.

Optionally, the functional layer 300 includes a hole transporting layer 320 which containing the nitrogen-containing compound of the present disclosure. The hole transporting layer 320 may be composed of the nitrogen-containing compound provided by the present disclosure, or may be composed of the nitrogen-containing compound provided by the present disclosure together with other materials.

Optionally, the hole transporting layer 320 may further include an inorganic doping material to improve the hole transport properties of the hole transporting layer 320.

According to one exemplary embodiment, as shown in FIG. 2, the photoelectric conversion device may include an anode 100, a hole transporting layer 320, a photoelectric conversion layer 360, an electron transporting layer 340, and a cathode 200 which are stacked in sequence.

Optionally, the photoelectric conversion device may be a solar cell, and in particular may be an organic thin film solar cell. For example, in one embodiment of the present disclosure, the solar cell may include an anode, a hole transporting layer, a photoelectric conversion layer, an electron transporting layer, and a cathode which are stacked in sequence, and the hole transporting layer contains the nitrogen-containing compound of the present disclosure.

The present disclosure also provides an electronic device, including the electronic element described above.

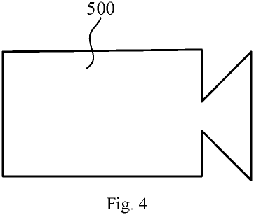

According to one embodiment, as shown in FIG. 3, the electronic device is a first electronic device 400 including the organic electroluminescent device described above. The first electronic device 400 may, for example, be a display device, a lighting device, an optical communication device, or other types of electronic devices, and may include, for example, but is not limited to, a computer screen, a mobile phone screen, a television, electronic paper, an emergency lighting lamp, an optical module, and the like.

According to another embodiment, as shown in FIG. 4, the electronic device is a second electronic device 500 including the photoelectric conversion device described above. The second electronic device 500 may, for example, be a solar power plant, a light detector, a fingerprint recognition device, an optical module, a CCD camera, or other types of electronic devices.

The present disclosure is further described in detail by synthesis examples and examples. However, the following embodiments are merely illustrative of the present disclosure, and are not intended to limit the present disclosure.

Synthesis examples are used to illustrate the synthesis of compounds.

Reactants used to synthesize the compounds of the present disclosure may include intermediates IM 1-I, IM 2-I, and IM 3-I of which structures are shown below;

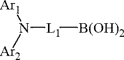

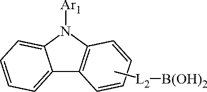

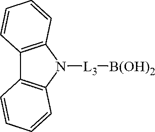

The above reactants can be commercially obtained, and can also be obtained by a method well known in the art, and may be synthesized, for example by reference to the documents KR1020140082486A, WO2014081206A1, and WO2012015274A2, and specific methods for the synthesis of the above reactants are well known in the art and will not be repeated here.

In synthesis examples, the used IM 1-I includes the following intermediates IM 1-1 to IM 1-9:

| | | |
|---|---|---|
| 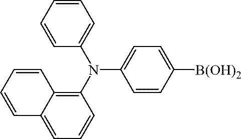 [SMILES: OB(O)c1ccc(N(c2ccccc2)c2cccc3ccccc23)cc1] | 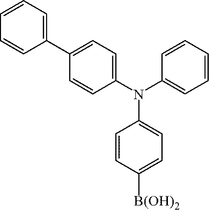 [SMILES: OB(O)c1ccc(N(c2ccccc2)c2ccc(-c3ccccc3)cc2)cc1] | 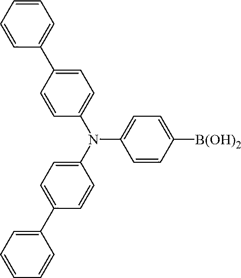 [SMILES: OB(O)c1ccc(N(c2ccc(-c3ccccc3)cc2)c2ccc(-c3ccccc3)cc2)cc1] |
| IM 1-1 | IM 1-2 | IM 1-3 |
| 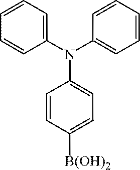 [SMILES: OB(O)c1ccc(N(c2ccccc2)c2ccccc2)cc1] | 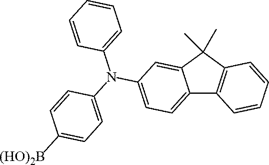 [SMILES: CC1(C)c2ccccc2-c2ccc(N(c3ccccc3)c3ccc(B(O)O)cc3)cc21] | 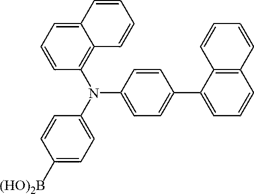 [SMILES: OB(O)c1ccc(N(c2ccc(-c3cccc4ccccc34)cc2)c2cccc3ccccc23)cc1] |
| IM 1-4 | IM 1-5 | IM 1-6 |
| 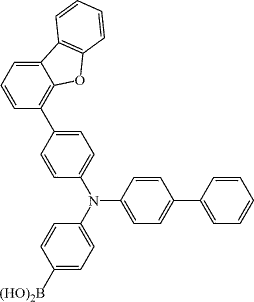 [SMILES: OB(O)c1ccc(N(c2ccc(-c3ccccc3)cc2)c2ccc(-c3cccc4c3oc3ccccc34)cc2)cc1] | 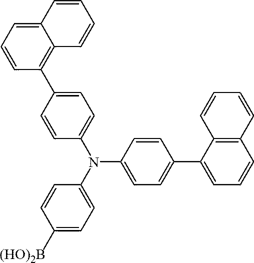 [SMILES: OB(O)c1ccc(N(c2ccc(-c3cccc4ccccc34)cc2)c2ccc(-c3cccc4ccccc34)cc2)cc1] | 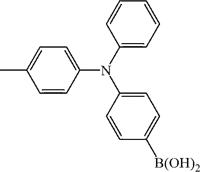 [SMILES: Cc1ccc(N(c2ccccc2)c2ccc(B(O)O)cc2)cc1] |
| IM 1-7 | IM 1-8 | IM 1-9 |

The intermediate IM 2-I including IM 2-1 to IM 2-11 are below:

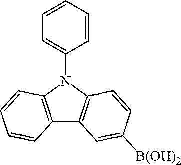 [SMILES: OB(O)c1ccc2c(c1)c1ccccc1n2-c1ccccc1]

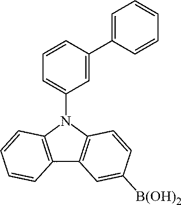 [SMILES: OB(O)c1ccc2c(c1)c1ccccc1n2-c1cccc(-c2ccccc2)c1]

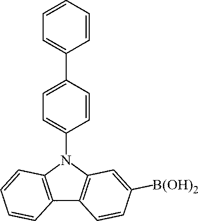 [SMILES: OB(O)c1ccc2c3ccccc3n(-c3ccc(-c4ccccc4)cc3)c2c1]

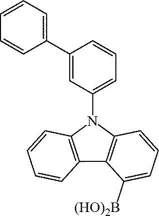 [SMILES: OB(O)c1cccc2c1c1ccccc1n2-c1cccc(-c2ccccc2)c1]

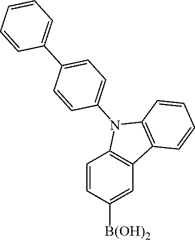 [SMILES: OB(O)c1ccc2c(c1)c1ccccc1n2-c1ccc(-c2ccccc2)cc1]

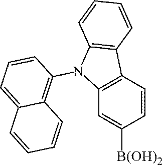 [SMILES: OB(O)c1ccc2c3ccccc3n(-c3cccc4ccccc34)c2c1]

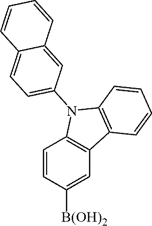 [SMILES: OB(O)c1ccc2c(c1)c1ccccc1n2-c1ccc2ccccc2c1]

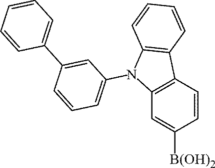 [SMILES: OB(O)c1ccc2c3ccccc3n(-c3cccc(-c4ccccc4)c3)c2c1]

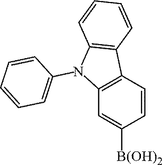 [SMILES: OB(O)c1ccc2c3ccccc3n(-c3ccccc3)c2c1]

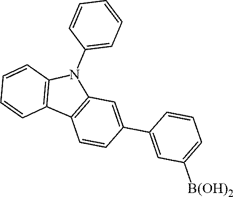 [SMILES: OB(O)c1cccc(-c2ccc3c4ccccc4n(-c4ccccc4)c3c2)c1]

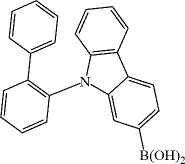 [SMILES: OB(O)c1ccc2c3ccccc3n(-c3ccccc3-c3ccccc3)c2c1]

The intermediate IM 3-I includes IM 3-1 to IM 3-4:

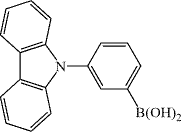 [SMILES: OB(O)c1cccc(-n2c3ccccc3c3ccccc32)c1]

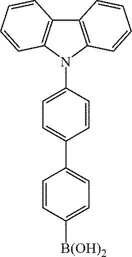 [SMILES: OB(O)c1ccc(-c2ccc(-n3c4ccccc4c4ccccc43)cc2)cc1]

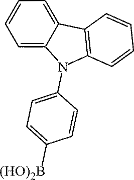 [SMILES: OB(O)c1ccc(-n2c3ccccc3c3ccccc32)cc1]

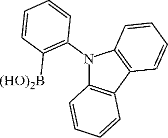 [SMILES: OB(O)c1ccccc1-n1c2ccccc2c2ccccc21]

### Synthesis Example 1

A compound A1 was synthesized by using the following method:

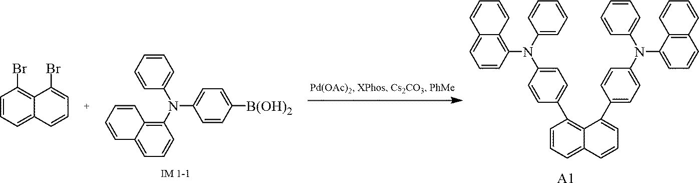

1,8-dibromonaphthalene (2.0 g; 7.0 mmol), IM 1-1 (7.1 g; 21.0 mmol), palladium acetate (0.3 g; 1.4 mmol), 2-dicyclohexylphosphino-2,4,6-triisopropylbiphenyl (0.7 g; 1.4 mmol), cesium carbonate (6.8 g; 21.0 mmol), and toluene (30 mL) were added into a 100 mL round-bottom flask which was dried and replaced with nitrogen, heated to 105° C. to 110° C. under stirring, and maintained for 18 h; the resulting reaction mixture was then cooled down to room temperature, after that deionized water (90 mL) was added and stirred for 30 min. Then an organic phase was separated and obtained. The obtained organic phase was dried with anhydrous magnesium sulfate, and was removed solvent under reduced pressure to obtain the crude product; and the obtained crude product was purified by silica gel column chromatography using dichloromethane/n-heptane in a volume ratio of 1:3 as a mobile phase. Finally the compound A1 (1.9 g; yield: 38%) which as a white solid was obtained.

### Synthesis Examples 2 to 9

Compounds in Table 1 were synthesized with reference to the method which was used to synthesize the compound A1, the difference is that reactants A in the table below were used to replace IM 1-1:

TABLE 1

| Synthesis example No. | Compound | Reactant A | Structure | Compound yield |
|---|---|---|---|---|
| 2 | A3 | 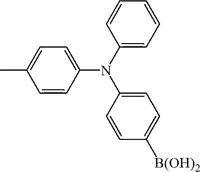 [SMILES: Cc1ccc(N(c2ccccc2)c2ccc(B(O)O)cc2)cc1] IM 1-9 | 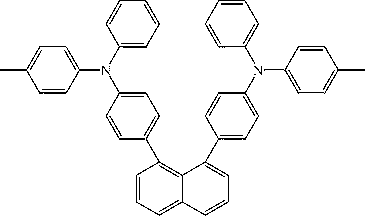 | 50% |
| 3 | A21 | 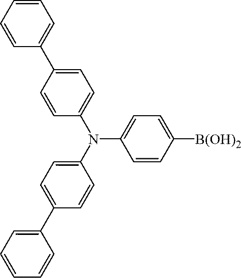 [SMILES: OB(O)c1ccc(N(c2ccc(-c3ccccc3)cc2)c2ccc(-c3ccccc3)cc2)cc1] IM 1-3 | 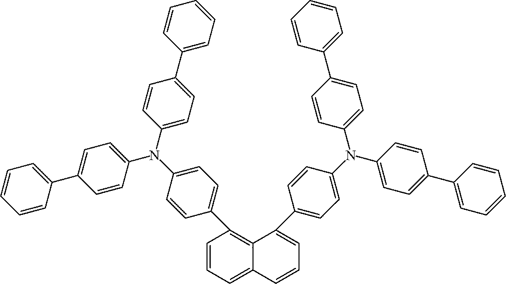 [SMILES: c1ccc(-c2ccc(N(c3ccc(-c4ccccc4)cc3)c3ccc(-c4cccc5cccc(-c6ccc(N(c7ccc(-c8ccccc8)cc7)c7ccc(-c8ccccc8)cc7)cc6)c45)cc3)cc2)cc1] | 45% |
| 4 | B1 | 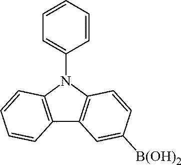 [SMILES: OB(O)c1ccc2c(c1)c1ccccc1n2-c1ccccc1] IM 2-1 | 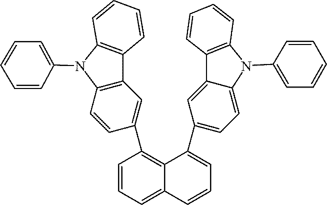 [SMILES: c1ccc(-n2c3ccccc3c3cc(-c4cccc5cccc(-c6ccc7c(c6)c6ccccc6n7-c6ccccc6)c45)ccc32)cc1] | 39% |
| 5 | B5 | 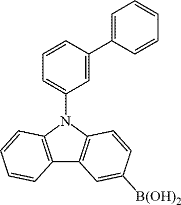 [SMILES: OB(O)c1ccc2c(c1)c1ccccc1n2-c1cccc(-c2ccccc2)c1] IM 2-2 | 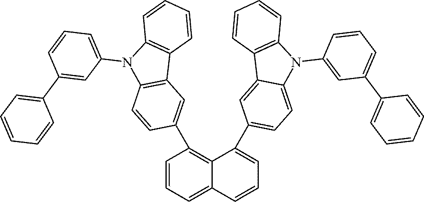 [SMILES: c1ccc(-c2cccc(-n3c4ccccc4c4cc(-c5cccc6cccc(-c7ccc8c(c7)c7ccccc7n8-c7cccc(-c8ccccc8)c7)c56)ccc43)c2)cc1] | 32% |
| 6 | B10 | 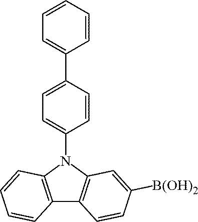 [SMILES: OB(O)c1ccc2c3ccccc3n(-c3ccc(-c4ccccc4)cc3)c2c1] IM 2-3 | 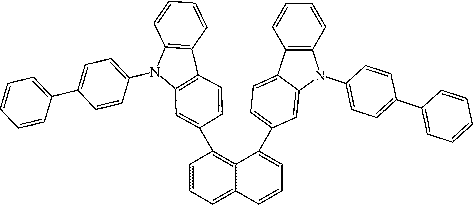 [SMILES: c1ccc(-c2ccc(-n3c4ccccc4c4ccc(-c5cccc6cccc(-c7ccc8c9ccccc9n(-c9ccc(-c%10ccccc%10)cc9)c8c7)c56)cc43)cc2)cc1] | 45% |
| 7 | B16 | 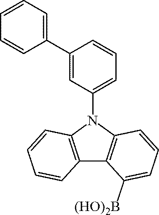 [SMILES: OB(O)c1cccc2c1c1ccccc1n2-c1cccc(-c2ccccc2)c1] IM 2-4 | 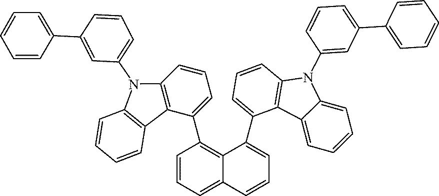 [SMILES: c1ccc(-c2cccc(-n3c4ccccc4c4c(-c5cccc6cccc(-c7cccc8c7c7ccccc7n8-c7cccc(-c8ccccc8)c7)c56)cccc43)c2)cc1] | 19% |
| 8 | B18 | 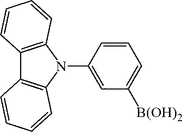 [SMILES: OB(O)c1cccc(-n2c3ccccc3c3ccccc32)c1] IM 3-1 | 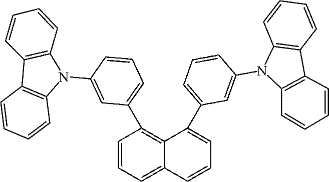 [SMILES: c1cc(-c2cccc3cccc(-c4cccc(-n5c6ccccc6c6ccccc65)c4)c23)cc(-n2c3ccccc3c3ccccc32)c1] | 43% |
| 9 | B23 | 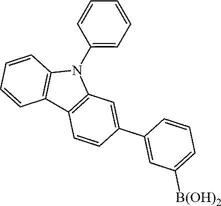 [SMILES: OB(O)c1cccc(-c2ccc3c4ccccc4n(-c4ccccc4)c3c2)c1] IM 2-10 | 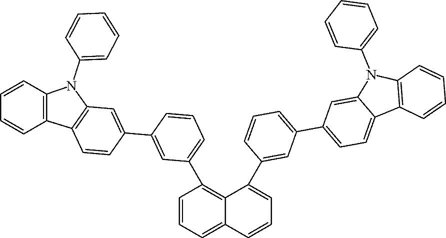 [SMILES: c1ccc(-n2c3ccccc3c3ccc(-c4cccc(-c5cccc6cccc(-c7cccc(-c8ccc9c%10ccccc%10n(-c%10ccccc%10)c9c8)c7)c56)c4)cc32)cc1] | 55% |

### Synthesis Examples 10 to 20

### 1. Synthesis of Intermediates IM a to IM K

1) IM a was synthesized by using the following method:

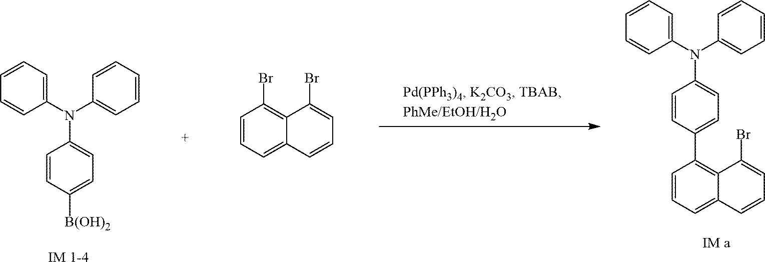

An intermediate IM 1-4 (10.0 g; 34.6 mmol), 1,8-dibromonaphthalene (10.9 g; 38.0 mmol), tetrakis(triphenylphosphine)palladium (0.8 g; 0.7 mmol), tetrabutylammonium bromide (2.2 g; 6.9 mmol), potassium carbonate (7.2 g; 51.9 mmol), toluene (100 mL), ethanol (20 mL), and deionized water (20 mL) were added into a 250 mL round-bottom flask which was dried and replaced with nitrogen, heated to 75° C. to 80° C. under stirring, and maintained for 12 h; the resulting reaction mixture was then cooled down to room temperature, after that deionized water (200 mL) was added and stirred for 10 min. Then an organic phase was separated and obtained, the obtained organic phase was dried with anhydrous magnesium sulfate, and was removed solvent under reduced pressure to obtain the crude product; and the obtained crude product was purified by silica gel column chromatography using dichloromethane/n-heptane in a volume ratio of 1:5 as a mobile phase. Finally the intermediate IM a (11.7 g; yield: 75%) which as a white solid was obtained.

2) Intermediates in Table 2 were synthesized with reference to the same method as that for the intermediate IM a, the difference is that reactants B in Table 2 were used to replace the intermediate IM 1-4:

TABLE 2

| Intermediate No. | Reactant B | Intermediate structure | Intermediate yield |
|---|---|---|---|
| IM b | 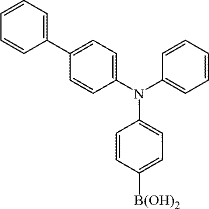 [SMILES: OB(O)c1ccc(N(c2ccccc2)c2ccc(-c3ccccc3)cc2)cc1] IM 1-2 | 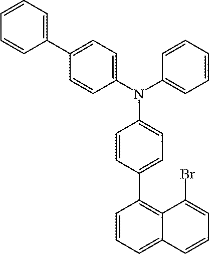 [SMILES: Brc1cccc2cccc(-c3ccc(N(c4ccccc4)c4ccc(-c5ccccc5)cc4)cc3)c12] | 77% |
| IM c | 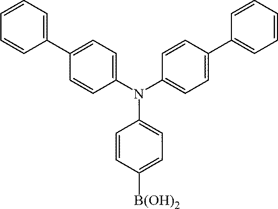 [SMILES: OB(O)c1ccc(N(c2ccc(-c3ccccc3)cc2)c2ccc(-c3ccccc3)cc2)cc1] IM 1-3 | 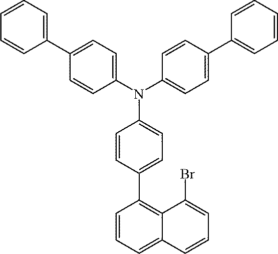 [SMILES: Brc1cccc2cccc(-c3ccc(N(c4ccc(-c5ccccc5)cc4)c4ccc(-c5ccccc5)cc4)cc3)c12] | 82% |
| IM d | 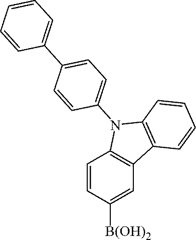 [SMILES: OB(O)c1ccc2c(c1)c1ccccc1n2-c1ccc(-c2ccccc2)cc1] IM 2-5 | 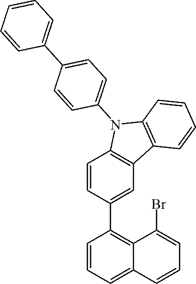 [SMILES: Brc1cccc2cccc(-c3ccc4c(c3)c3ccccc3n4-c3ccc(-c4ccccc4)cc3)c12] | 60% |
| IM e | 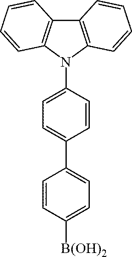 [SMILES: OB(O)c1ccc(-c2ccc(-n3c4ccccc4c4ccccc43)cc2)cc1] IM 3-2 | 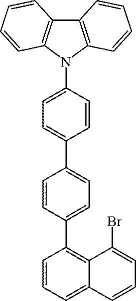 [SMILES: Brc1cccc2cccc(-c3ccc(-c4ccc(-n5c6ccccc6c6ccccc65)cc4)cc3)c12] | 80% |
| IM f | 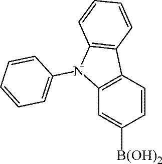 [SMILES: OB(O)c1ccc2c3ccccc3n(-c3ccccc3)c2c1] IM 2-9 | 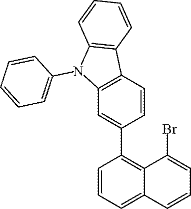 [SMILES: Brc1cccc2cccc(-c3ccc4c5ccccc5n(-c5ccccc5)c4c3)c12] | 69% |
| IM g | 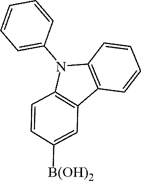 [SMILES: OB(O)c1ccc2c(c1)c1ccccc1n2-c1ccccc1] IM 2-1 | 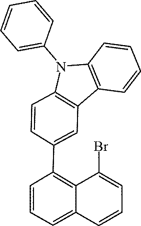 [SMILES: Brc1cccc2cccc(-c3ccc4c(c3)c3ccccc3n4-c3ccccc3)c12] | 70% |
| IM h | 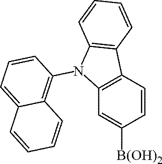 [SMILES: OB(O)c1ccc2c3ccccc3n(-c3cccc4ccccc34)c2c1] IM 2-6 | 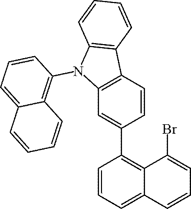 [SMILES: Brc1cccc2cccc(-c3ccc4c5ccccc5n(-c5cccc6ccccc56)c4c3)c12] | 55% |
| IM i | 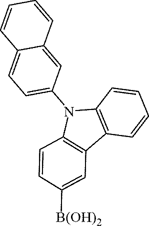 [SMILES: OB(O)c1ccc2c(c1)c1ccccc1n2-c1ccc2ccccc2c1] IM 2-7 | 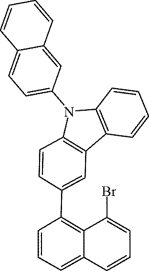 [SMILES: Brc1cccc2cccc(-c3ccc4c(c3)c3ccccc3n4-c3ccc4ccccc4c3)c12] | 74% |
| IM j | 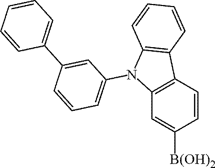 [SMILES: OB(O)c1ccc2c3ccccc3n(-c3cccc(-c4ccccc4)c3)c2c1] IM 2-8 | 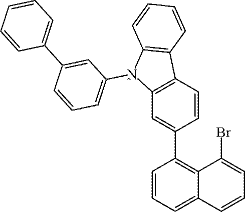 [SMILES: Brc1cccc2cccc(-c3ccc4c5ccccc5n(-c5cccc(-c6ccccc6)c5)c4c3)c12] | 58% |
| IM k | 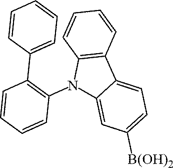 [SMILES: OB(O)c1ccc2c3ccccc3n(-c3ccccc3-c3ccccc3)c2c1] IM 2-11 | 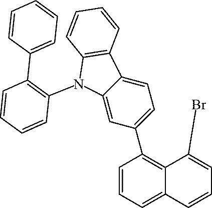 [SMILES: Brc1cccc2cccc(-c3ccc4c5ccccc5n(-c5ccccc5-c5ccccc5)c4c3)c12] | 32% |

### Synthesis Example 10

A compound A30 was synthesized by using the following method:

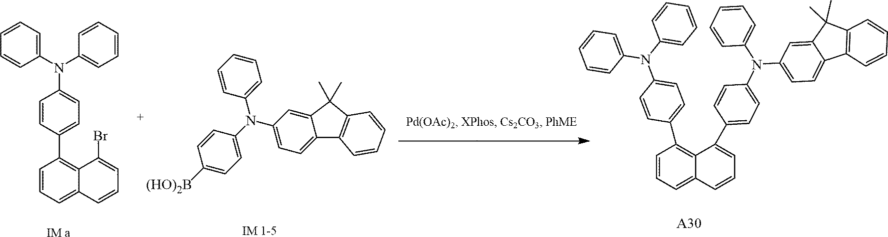

The intermediate IM a (3.0 g; 6.7 mmol), IM 1-5 (4.0 g; 10.0 mmol), palladium acetate (0.3 g; 1.3 mmol), 2-dicyclohexylphosphino-2,4,6-triisopropylbiphenyl (0.6 g; 1.3 mmol), cesium carbonate (3.3 g; 10.0 mmol), and toluene (40 mL) were added into a 100 mL round-bottom flask which was dried and replaced with nitrogen, heated to 105° C. to 110° C. under stirring, and maintained for 16 h; the resulting reaction mixture was then cooled down to room temperature, after that deionized water (100 mL) was added and stirred for 30 min. Then an organic phase was separated and obtained. The obtained organic phase was dried with anhydrous magnesium sulfate, and was removed solvent under reduced pressure to obtain the crude product; and the obtained crude product was purified by silica gel column chromatography using dichloromethane/n-heptane in a volume ratio of 1:2 as a mobile phase. Finally the compound A30 (2.0 g; yield: 41%) which as a white solid was obtained.

### Synthesis Examples 11 to 20

Compounds shown in Table 3 were synthesized with reference to the method for the compound A30 except that: reactants C in the table below were used to replace the intermediate IM a, and reactants D in the table below were used to replace IM 1-5, so as to synthesize the compounds in Table 3:

TABLE 3

| Synthesis example No. | Compound | Reactant C | Reactant D | Structure | Compound yield |
|---|---|---|---|---|---|
| 11 | A36 | 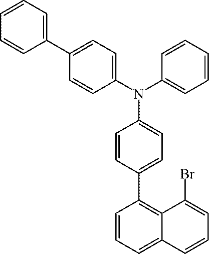 [SMILES: Brc1cccc2cccc(-c3ccc(N(c4ccccc4)c4ccc(-c5ccccc5)cc4)cc3)c12] IM b | 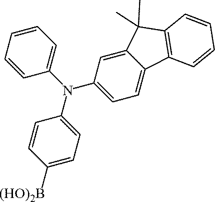 [SMILES: CC1(C)c2ccccc2-c2ccc(N(c3ccccc3)c3ccc(B(O)O)cc3)cc21] IM 1-5 | 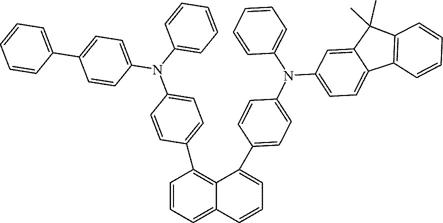 [SMILES: CC1(C)c2ccccc2-c2ccc(N(c3ccccc3)c3ccc(-c4cccc5cccc(-c6ccc(N(c7ccccc7)c7ccc(-c8ccccc8)cc7)cc6)c45)cc3)cc21] | 33% |
| 12 | A39 | 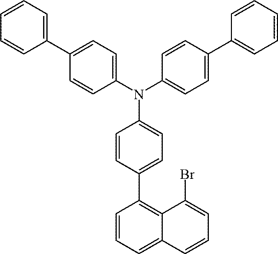 [SMILES: Brc1cccc2cccc(-c3ccc(N(c4ccc(-c5ccccc5)cc4)c4ccc(-c5ccccc5)cc4)cc3)c12] IM c | 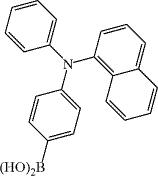 [SMILES: OB(O)c1ccc(N(c2ccccc2)c2cccc3ccccc23)cc1] IM 1-1 | 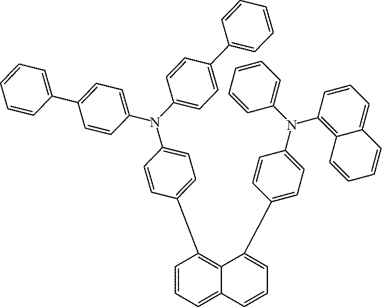 [SMILES: c1ccc(-c2ccc(N(c3ccc(-c4ccccc4)cc3)c3ccc(-c4cccc5cccc(-c6ccc(N(c7ccccc7)c7cccc8ccccc78)cc6)c45)cc3)cc2)cc1] | 27% |
| 13 | B28 | 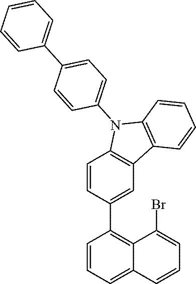 [SMILES: Brc1cccc2cccc(-c3ccc4c(c3)c3ccccc3n4-c3ccc(-c4ccccc4)cc3)c12] IM d | 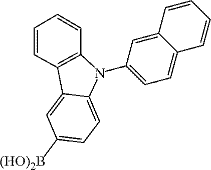 [SMILES: OB(O)c1ccc2c(c1)c1ccccc1n2-c1ccc2ccccc2c1] IM 2-7 | 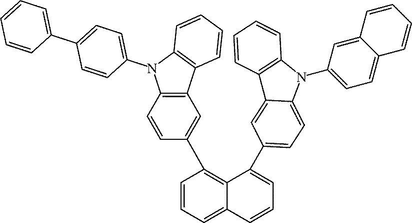 [SMILES: c1ccc(-c2ccc(-n3c4ccccc4c4cc(-c5cccc6cccc(-c7ccc8c(c7)c7ccccc7n8-c7ccc8ccccc8c7)c56)ccc43)cc2)cc1] | 25% |
| 14 | B41 | 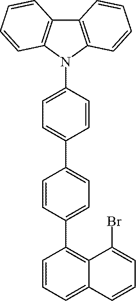 [SMILES: Brc1cccc2cccc(-c3ccc(-c4ccc(-n5c6ccccc6c6ccccc65)cc4)cc3)c12] IM e | 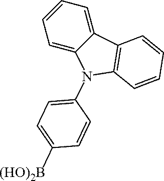 [SMILES: OB(O)c1ccc(-n2c3ccccc3c3ccccc32)cc1] IM 3-3 | 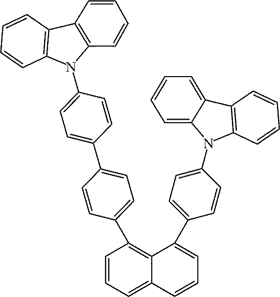 [SMILES: c1cc(-c2ccc(-c3ccc(-n4c5ccccc5c5ccccc54)cc3)cc2)c2c(-c3ccc(-n4c5ccccc5c5ccccc54)cc3)cccc2c1] | 45% |
| 15 | C3 | 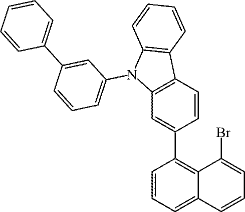 [SMILES: Brc1cccc2cccc(-c3ccc4c5ccccc5n(-c5cccc(-c6ccccc6)c5)c4c3)c12] IM j | 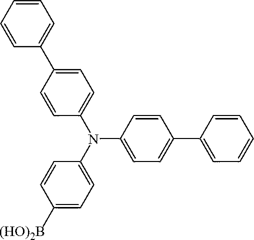 [SMILES: OB(O)c1ccc(N(c2ccc(-c3ccccc3)cc2)c2ccc(-c3ccccc3)cc2)cc1] IM 1-3 | 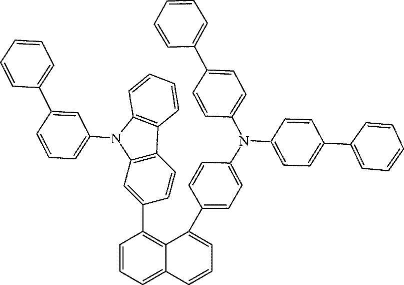 [SMILES: c1ccc(-c2ccc(N(c3ccc(-c4ccccc4)cc3)c3ccc(-c4cccc5cccc(-c6ccc7c8ccccc8n(-c8cccc(-c9ccccc9)c8)c7c6)c45)cc3)cc2)cc1] | 30% |
| 16 | C5 | 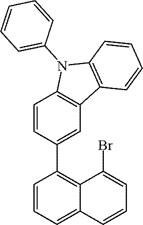 [SMILES: Brc1cccc2cccc(-c3ccc4c(c3)c3ccccc3n4-c3ccccc3)c12] IM g | 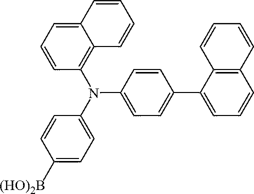 [SMILES: OB(O)c1ccc(N(c2ccc(-c3cccc4ccccc34)cc2)c2cccc3ccccc23)cc1] IM 1-6 | 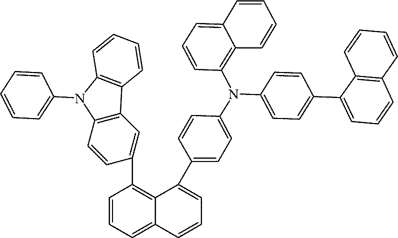 [SMILES: c1ccc(-n2c3ccccc3c3cc(-c4cccc5cccc(-c6ccc(N(c7ccc(-c8cccc9ccccc89)cc7)c7cccc8ccccc78)cc6)c45)ccc32)cc1] | 18% |
| 17 | C10 | 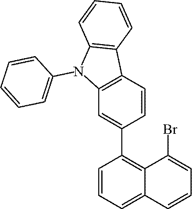 [SMILES: Brc1cccc2cccc(-c3ccc4c5ccccc5n(-c5ccccc5)c4c3)c12] IM f | 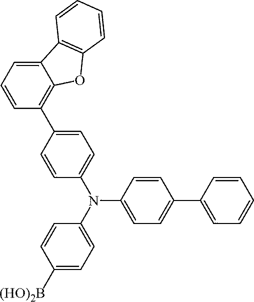 [SMILES: OB(O)c1ccc(N(c2ccc(-c3ccccc3)cc2)c2ccc(-c3cccc4c3oc3ccccc34)cc2)cc1] IM 1-7 | 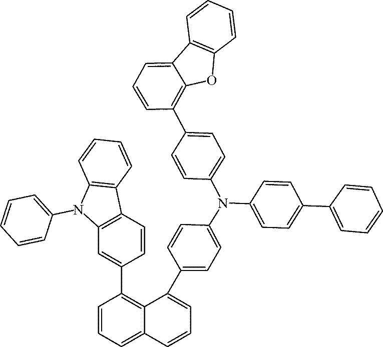 [SMILES: c1ccc(-c2ccc(N(c3ccc(-c4cccc5c4oc4ccccc45)cc3)c3ccc(-c4cccc5cccc(-c6ccc7c8ccccc8n(-c8ccccc8)c7c6)c45)cc3)cc2)cc1] | 25% |
| 18 | C12 | 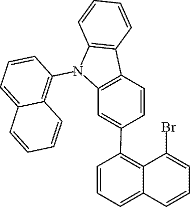 [SMILES: Brc1cccc2cccc(-c3ccc4c5ccccc5n(-c5cccc6ccccc56)c4c3)c12] IM h | 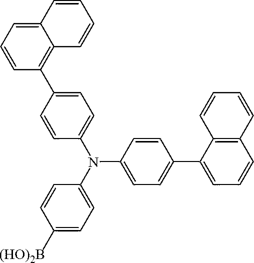 [SMILES: OB(O)c1ccc(N(c2ccc(-c3cccc4ccccc34)cc2)c2ccc(-c3cccc4ccccc34)cc2)cc1] IM 1-8 | 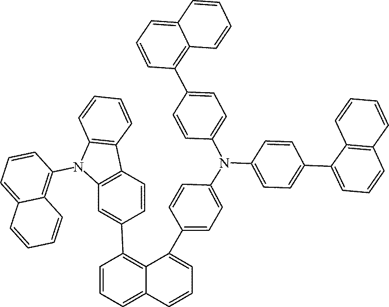 [SMILES: c1ccc2c(-c3ccc(N(c4ccc(-c5cccc6ccccc56)cc4)c4ccc(-c5cccc6cccc(-c7ccc8c9ccccc9n(-c9cccc%10ccccc9%10)c8c7)c56)cc4)cc3)cccc2c1] | 26% |
| 19 | C13 | 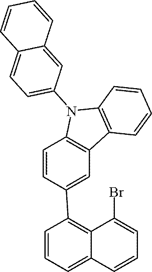 [SMILES: Brc1cccc2cccc(-c3ccc4c(c3)c3ccccc3n4-c3ccc4ccccc4c3)c12] IM i | 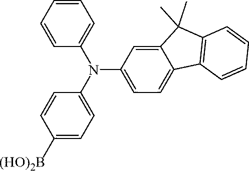 [SMILES: CC1(C)c2ccccc2-c2ccc(N(c3ccccc3)c3ccc(B(O)O)cc3)cc21] IM 1-5 | 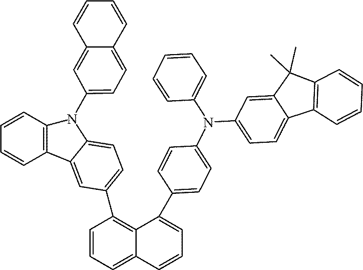 [SMILES: CC1(C)c2ccccc2-c2ccc(N(c3ccccc3)c3ccc(-c4cccc5cccc(-c6ccc7c(c6)c6ccccc6n7-c6ccc7ccccc7c6)c45)cc3)cc21] | 39% |
| 20 | B46 | 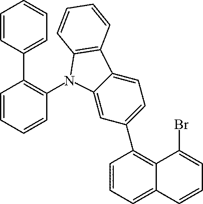 [SMILES: Brc1cccc2cccc(-c3ccc4c5ccccc5n(-c5ccccc5-c5ccccc5)c4c3)c12] IM k | 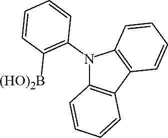 [SMILES: OB(O)c1ccccc1-n1c2ccccc2c2ccccc21] IM 3-4 | 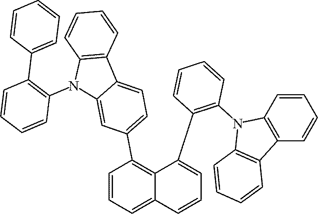 [SMILES: c1ccc(-c2ccccc2-n2c3ccccc3c3ccc(-c4cccc5cccc(-c6ccccc6-n6c7ccccc7c7ccccc76)c45)cc32)cc1] | 25% |

The above synthesized compounds were analyzed by mass spectrometry, and the obtained data are shown below:

| | |
|---|---|
| Compound A1: m/z=715.3 (M+H)⁺ | Compound B16: m/z=763.3 (M+H)⁺ |
| Compound A3: m/z=643.3 (M+H)⁺ | Compound B18: m/z=611.2 (M+H)⁺ |
| Compound A21: m/z=919.4 (M+H)⁺ | Compound B28: m/z=737.3 (M+H)⁺ |
| Compound A30: m/z=731.3 (M+H)⁺ | Compound B41: m/z=687.3 (M+H)⁺ |
| Compound A36: m/z=807.4 (M+H)⁺ | Compound C3: m/z=841.4 (M+H)⁺ |
| Compound A39: m/z=817.4 (M+H)⁺ | Compound C5: m/z=789.3 (M+H)⁺ |
| Compound B1: m/z=611.2 (M+H)⁺ | Compound C10: m/z=855.3 (M+H)⁺ |
| Compound B5: m/z=763.3 (M+H)⁺ | Compound C12: m/z=915.4 (M+H)⁺ |
| Compound B10: m/z=763.3 (M+H)⁺ | Compound C13: m/z=779.3 (M+H)⁺ |
| Compound B23: m/z=763.3 (M+H)⁺ | Compound B46: m/z=687.3 (M+H)⁺ |

NMR data for a compound A1:

¹H NMR (CD₂Cl₂, 400 MHz): 7.82 (d, 2H), 7.77 (d, 2H), 7.71 (d, 2H), 7.54 (d, 2H), 7.48 (t, 2H), 7.38 (t, 2H), 7.34-7.29 (m, 4H), 7.25 (t, 2H), 7.15 (d, 2H), 7.05 (t, 4H), 7.01 (t, 4H), 6.95 (d, 4H), 6.81-6.77 (m, 6H).

NMR data for a compound B28:

¹H NMR (CD₂Cl₂, 400 MHz): 8.03-7.99 (m, 2H), 7.95 (d, 1H), 7.91-7.88 (m, 3H), 7.79 (d, 2H), 7.69-7.66 (m, 2H), 7.64-7.58 (m, 4H), 7.57-7.53 (m, 5H), 7.51-7.46 (m, 5H), 7.43 (t, 1H), 7.38-7.32 (m, 3H), 7.24-7.18 (m, 6H), 7.05-7.01 (m, 2H).

### Manufacture and Evaluation of Organic Electroluminescent Device

### Examples 1 To 11: Manufacture And Evaluation Of Blue Organic Electroluminescent Device

### Example 1

An anode was prepared by the following process: an ITO substrate (manufactured by Corning) with a thickness of 1500 Å was cut into a dimension of 40 mm×40 mm×0.7 mm so as to be prepared into an experimental substrate with cathode pattern, anode pattern and insulating layer pattern by photoetching process, and surface treatment was performed by utilizing ultraviolet ozone and O₂:N₂ plasma so as to increase the work function of the anode (of the experimental substrate) and remove scum.

F4-TCNQ was vacuum-evaporated on the experimental substrate (the anode) to form a hole injection layer (HIL) with a thickness of 100 Å, and a compound A1 was evaporated on the hole injection layer to form a first hole transporting layer (HTL1) with a thickness of 800 Å.

A compound HT-02 was vacuum-evaporated on the first hole transporting layer to form a second hole transporting layer (HTL2) with a thickness of 100 Å.

BH-01 and BD-01 were co-evaporated on the second hole transporting layer at an evaporation rate ratio of 98%:2% to form a blue luminescence layer (EML) with a thickness of 200 Å.

ET-01 and LiQ were mixed at a weight ratio of 1:1 and evaporated to form an electron transporting layer (ETL) with a thickness of 300 Å, LiQ was evaporated on the electron transporting layer to form an electron injection layer (EIL) with a thickness of 10 Å, and then magnesium (Mg) and silver (Ag) were vacuum-evaporated with mixed on the electron injection layer at an evaporation rate ratio of 1:9 to form a cathode with a thickness of 105 Å.

In addition, CP-01 was evaporated on the cathode to form an organic capping layer (CPL) with a thickness of 650 Å, thus the organic luminescence device is completely manufactured.

### Examples 2 to 6

An organic electroluminescent device was manufactured by the same method as that in Example 1, except that compounds shown in the following Table 4 were used to replace the compound A1 when the first hole transporting layer was formed.

### Comparative Examples 1 to 2

An organic electroluminescent device was manufactured by the same method as that in Example 1 except that HT-01 and a compound a were respectively used in Comparative examples 1 and 2 to replace the compound A1 when the first hole transporting layer was formed.

### Examples 7 to 11

An organic electroluminescent device was manufactured by the same method as that in Example 1 except that: a compound HT-01 was used to replace the compound A1 when the first hole transporting layer was formed, and compounds in Table 4 were used to replace HT-02 when the second hole transporting layer was formed.

### Comparative Example 3

An organic electroluminescent device was manufactured by the same method as that in Example 1 except that: the compound HT-01 was used to replace the compound A1 when the first hole transporting layer was formed, and a compound b was used to replace HT-02 when the second hole transporting layer was formed.

The structures of main materials used in the above examples and comparative examples are shown as below:

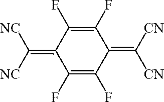 [SMILES: N#CC(C#N)=c1c(F)c(F)c(=C(C#N)C#N)c(F)c1F]

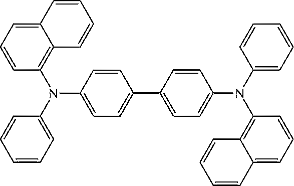 [SMILES: c1ccc(N(c2ccc(-c3ccc(N(c4ccccc4)c4cccc5ccccc45)cc3)cc2)c2cccc3ccccc23)cc1]

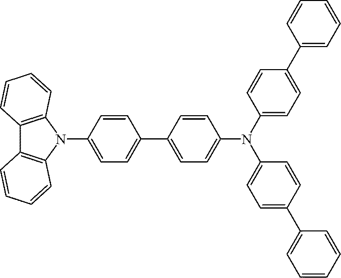 [SMILES: c1ccc(-c2ccc(N(c3ccc(-c4ccccc4)cc3)c3ccc(-c4ccc(-n5c6ccccc6c6ccccc65)cc4)cc3)cc2)cc1]

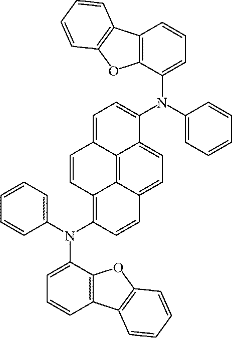 [SMILES: c1ccc(N(c2ccc3ccc4c(N(c5ccccc5)c5cccc6c5oc5ccccc56)ccc5ccc2c3c54)c2cccc3c2oc2ccccc23)cc1]

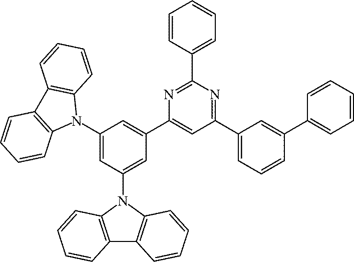 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

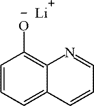 [SMILES: [Li+] ; [O-]c1cccc2cccnc12]

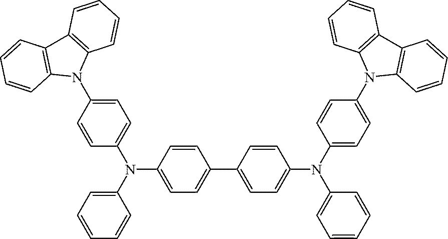 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]

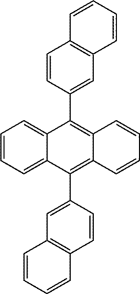 [SMILES: c1ccc2cc(-c3c4ccccc4c(-c4ccc5ccccc5c4)c4ccccc34)ccc2c1]

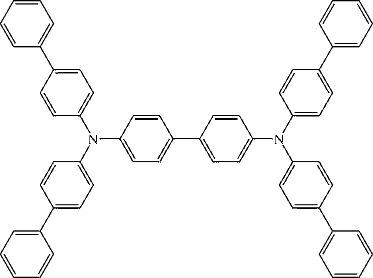 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

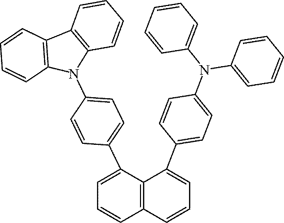 [SMILES: c1ccc(N(c2ccccc2)c2ccc(-c3cccc4cccc(-c5ccc(-n6c7ccccc7c7ccccc76)cc5)c34)cc2)cc1]

For the manufactured organic electroluminescent devices, the photoelectric performance of the device was analyzed under a condition of 10 mA/cm², and the service life performance of the device was analyzed under a condition of 20 mA/cm², and the results are shown in Table 4.

TABLE 4

| No. | HTL1 compound | HTL2 compound | Driving Voltage (V) | Current efficiency Cd/A | Power efficiency (lm/W) | CIE-x | CIE-y | External Quantum efficiency EQE% | T95 (h) |
|---|---|---|---|---|---|---|---|---|---|
| Example 1 | A1 | HT-02 | 3.68 | 6.38 | 5.45 | 0.140 | 0.050 | 13.1 | 145 |
| Example 2 | A3 | HT-02 | 3.64 | 6.34 | 5.47 | 0.140 | 0.050 | 13.0 | 120 |
| Example 3 | A21 | HT-02 | 3.68 | 6.43 | 5.49 | 0.140 | 0.050 | 13.2 | 141 |
| Example 4 | A30 | HT-02 | 3.70 | 6.40 | 5.43 | 0.140 | 0.050 | 13.2 | 147 |
| Example 5 | A36 | HT-02 | 3.64 | 6.32 | 5.45 | 0.140 | 0.050 | 13.0 | 139 |
| Example 6 | A39 | HT-02 | 3.67 | 6.45 | 5.52 | 0.140 | 0.050 | 13.3 | 144 |
| Example 7 | HT-01 | C3 | 3.92 | 6.51 | 5.22 | 0.140 | 0.050 | 13.4 | 131 |
| Example 8 | HT-01 | C5 | 3.89 | 6.59 | 5.32 | 0.140 | 0.050 | 13.6 | 139 |
| Example 9 | HT-01 | C10 | 3.90 | 6.56 | 5.28 | 0.140 | 0.050 | 13.5 | 130 |
| Example 10 | HT-01 | C12 | 3.87 | 6.49 | 5.27 | 0.140 | 0.050 | 13.3 | 145 |
| Example 11 | HT-01 | C13 | 3.86 | 6.60 | 5.37 | 0.140 | 0.050 | 13.6 | 133 |
| Comparative example 1 | HT-01 | HT-02 | 4.03 | 6.31 | 4.92 | 0.140 | 0.050 | 13.0 | 90 |
| Comparative example 2 | Compound a | HT-02 | 4.04 | 6.33 | 4.92 | 0.140 | 0.050 | 13.0 | 85 |
| Comparative example 3 | HT-01 | Compound b | 3.92 | 6.08 | 4.87 | 0.140 | 0.050 | 12.5 | 102 |

In connection with the above table, under the condition that other layers of the device are the same, by comparing Examples 1 to 6 with Comparative examples 1 to 2, it can be seen that when the compounds according to the present disclosure are used as a first hole transporting layer material, the driving voltage can be reduced, and the service life of the device can be increased; and by comparing Examples 7 to 11 with Comparative examples 1 and 3, it can be seen that when the compounds of the present disclosure are used as a second hole transporting layer material, both the luminescence efficiency and service life of the device can be simultaneously increased. Thus, when the compounds of the present disclosure are used for manufacturing the blue organic electroluminescent device, the service life of the organic electroluminescent device can be effectively prolonged, and the luminescence efficiency or the driving voltage is improved to a certain extent.

### Examples 12 To 20: Manufacture And Evaluation Of Green Organic Electroluminescent Device

### Example 12

An anode was prepared by the following process: an ITO substrate (manufactured by Corning) with a thickness of 1500 Å was cut into a dimension of 40 mm×40 mm×0.7 mm so as to be prepared into an experimental substrate with cathode pattern, anode pattern and insulating layer pattern by photoetching process, and surface treatment was performed by ultraviolet ozone and O₂:N₂ plasma so as to increase the work function of the anode (of the experimental substrate) and remove scum.

F4-TCNQ was vacuum-evaporated on the experimental substrate (the anode) to form a hole injection layer (HIL) with a thickness of 100 Å, and HT-01 was evaporated on the hole injection layer to form a first hole transporting layer with a thickness of 800 Å.

HT-02 was vacuum-evaporated on the first hole transporting layer to form a second hole transporting layer with a thickness of 300 Å.

Compound B1, GH-n1 and Ir(ppy)₃ were co-evaporated on the second hole transporting layer at an evaporation rate ratio of 50%:45%:5% to form a green luminescence layer (EML) with a thickness of 400 Å.

ET-01 and LiQ were mixed at a weight ratio of 1:1 and evaporated to form an electron transporting layer (ETL) with a thickness of 300 Å, LiQ was evaporated on the electron transporting layer to form an electron injection layer (EIL) with a thickness of 10 Å, and then magnesium (Mg) and silver (Ag) were vacuum-evaporated with mixed on the electron injection layer at an evaporation rate ratio of 1:9 to form a cathode with a thickness of 105 Å.

In addition, CP-01 with a thickness of 650 Å was evaporated on the cathode to form an organic capping layer (CPL), thus completing the manufacture of an organic luminescence device.

### Examples 13 to 20

An organic electroluminescent device was manufactured by the same method as that in Example 12 except that compounds shown in the following Table 5 were used to replace the compound B1 when the luminescence layer was formed (the compound B1 and the compounds for replacing the compound B1 are collectively referred to as an EM1 compound in Table 5).

### Comparative Examples 4 to 5

An organic electroluminescent device was manufactured by the same method as that in Example 12 except that a compound c and a compound d were respectively used in Comparative examples 4 and 5 to replace the compound B1 when the luminescence layer was formed.

The structures of materials used in the above examples and comparative examples are shown in the table as below:

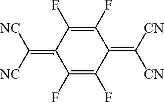 [SMILES: N#CC(C#N)=c1c(F)c(F)c(=C(C#N)C#N)c(F)c1F]

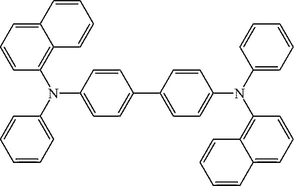 [SMILES: c1ccc(N(c2ccc(-c3ccc(N(c4ccccc4)c4cccc5ccccc45)cc3)cc2)c2cccc3ccccc23)cc1]

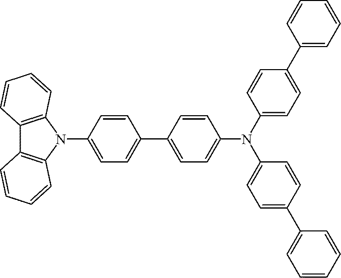 [SMILES: c1ccc(-c2ccc(N(c3ccc(-c4ccccc4)cc3)c3ccc(-c4ccc(-n5c6ccccc6c6ccccc65)cc4)cc3)cc2)cc1]

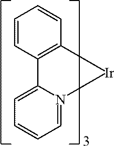

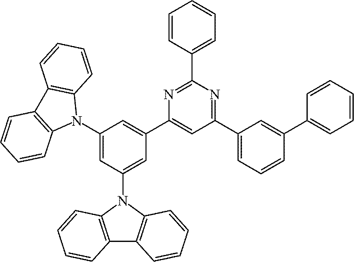 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

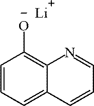 [SMILES: [Li+] ; [O-]c1cccc2cccnc12]

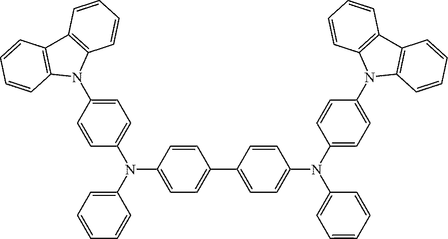 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]

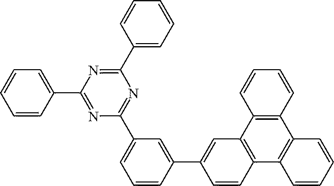 [SMILES: c1ccc(-c2nc(-c3ccccc3)nc(-c3cccc(-c4ccc5c6ccccc6c6ccccc6c5c4)c3)n2)cc1]

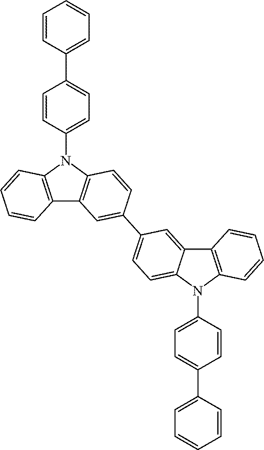 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

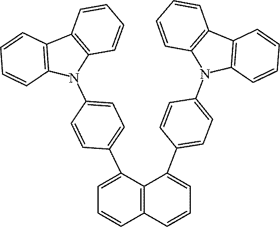 [SMILES: c1cc(-c2ccc(-n3c4ccccc4c4ccccc43)cc2)c2c(-c3ccc(-n4c5ccccc5c5ccccc54)cc3)cccc2c1]

For the manufactured organic electroluminescent devices, the photoelectric performance of the device was analyzed under a condition of 10 mA/cm², and the service life performance of the device was analyzed under a condition of 20 mA/cm², and the results are shown in the table as below:

TABLE 5

| No. | EM1 compound | Driving voltage (V) | Current efficiency (Cd/A) | Power efficiency (lm/W) | CIEx | ClEy | External quantum efficiency EQE (%) | T95 (h) |
|---|---|---|---|---|---|---|---|---|
| Example 12 | B1 | 3.83 | 83.8 | 68.7 | 0.220 | 0.730 | 20.1 | 246 |
| Example 13 | B5 | 3.85 | 84.7 | 69.1 | 0.220 | 0.730 | 20.3 | 262 |
| Example 14 | B10 | 3.80 | 81.6 | 67.4 | 0.220 | 0.730 | 19.6 | 273 |
| Example 15 | B16 | 3.84 | 81.3 | 66.5 | 0.220 | 0.730 | 19.5 | 268 |
| Example 16 | B18 | 3.82 | 83.1 | 68.4 | 0.220 | 0.730 | 20.0 | 230 |
| Example 17 | B28 | 3.85 | 81.7 | 66.7 | 0.220 | 0.730 | 19.6 | 284 |
| Example 18 | B41 | 3.82 | 82.5 | 67.9 | 0.220 | 0.730 | 19.8 | 271 |
| Example 19 | B23 | 3.78 | 82.8 | 68.8 | 0.220 | 0.730 | 19.9 | 290 |
| Example 20 | B46 | 3.86 | 83.3 | 67.8 | 0.220 | 0.730 | 20.0 | 251 |
| Comparative example 4 | c | 3.80 | 81.4 | 67.3 | 0.220 | 0.730 | 19.5 | 145 |
| Comparative example 5 | d | 3.80 | 74.1 | 61.3 | 0.220 | 0.730 | 17.8 | 172 |

In combination with the data in Table 5, it can be seen that, Examples 12 to 20 in which the compounds of the present disclosure are used as a mixed host material of the green luminescence layer, when compared with Comparative example 4, the service life of the device is greatly enhanced under the condition that the driving voltage and current efficiency levels are equivalent; and when compared with Comparative example 5, the current efficiency and the service life are also improved to a certain extent.

In summary, when the nitrogen-containing compound of the present disclosure is used for manufacturing the green organic electroluminescent device, the service life of the organic electroluminescent device can be effectively prolonged, and the organic electroluminescent device has both higher luminescence efficiency and lower driving voltage.

1. A nitrogen-containing compound, having the structure shown in formula 1: 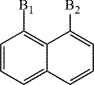
wherein B₁ and B₂ are the same as or different from each other, and are each independently selected from a group represented by formula 1-1, formula 1-2, or formula 1-3, and B₁ and B₂ are not a combination of the formula 1-1 and the formula 1-3: 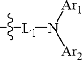 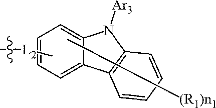 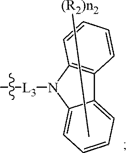
wherein [symbol] represents chemical bond;
Ar₁, Ar₂, and Ar₃ are the same as or different from each other, and are each independently selected from a substituted or unsubstituted aryl with a total number of carbon atoms being 6 to 30, or a substituted or unsubstituted heteroaryl with a total number of carbon atoms being 3 to 30;
L₁ is selected from a substituted or unsubstituted arylene with a total number of carbon atoms being 6 to 20, or a substituted or unsubstituted heteroarylene with a total number of carbon atoms being 4 to 15;
L₂ and L₃ are the same as or different from each other, and are each independently selected from single bond, a substituted or unsubstituted arylene with a total number of carbon atoms being 6 to 20, or a substituted or unsubstituted heteroarylene with a total number of carbon atoms being 4 to 15;
R₁ and R₂ are the same as or different from each other, and are each independently selected from deuterium, halogen group, cyano, an alkyl with 1 to 10 carbon atoms, a haloalkyl with 1 to 10 carbon atoms, an alkenyl with 2 to 6 carbon atoms, a cycloalkyl with 3 to 10 carbon atoms, a group A, a heteroaryl with 3 to 20 carbon atoms, an alkoxy with 1 to 10 carbon atoms, an alkylthio with 1 to 10 carbon atoms, or a trialkylsilyl with 3 to 12 carbon atoms; and the group A is selected from a substituted or unsubstituted aryl with a total number of carbon atoms being 6 to 20, and substituents in the aryl are each independently selected from deuterium, halogen group, cyano, an alkyl with 1 to 4 carbon atoms, a haloalkyl with 1 to 4 carbon atoms, a cycloalkyl with 3 to 10 carbon atoms, or a trialkylsilyl with 3 to 7 carbon atoms;
n₁ is the number of a substituent R₁, n₁ is selected from 0, 1, 2, 3, 4, 5, 6, or 7, when n₁ is greater than 1, any two R₁ are the same or different, and optionally, any two adjacent R₁ form a ring; and n₂ is the number of a substituent R₂, n₂ is selected from 0, 1, 2, 3, 4, 5, 6, 7 or 8, when n₂ is greater than 1, any two R₂ are the same or different, and optionally, any two adjacent R₂ form a ring;
substituents in Ar₁, Ar₂, and Ar₃ are the same or different, and are each independently selected from deuterium, halogen group, cyano, an aryl with 6 to 20 carbon atoms, a heteroaryl with 3 to 18 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
substituents in L₁, L₂, and L₃ are the same or different, and are each independently selected from deuterium, halogen group, cyano, an aryl with 6 to 12 carbon atoms, a heteroaryl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a trialkylsilyl with 3 to 12 carbon atoms;
optionally, any two adjacent substituents in the substituents of Ar₁, Ar₂, Ar₃, L₁, L₂ and L₃ form a ring; and,
B₁ and B₂ are not simultaneously 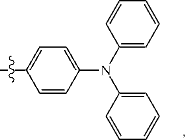
and when B₁ is selected from a structure of the formula 1-3, and L₃ is substituted or unsubstituted 1,4-phenylene, the structure of B₂ is different from that of B₁.

2. The nitrogen-containing compound of claim 1, wherein the nitrogen-containing compound is selected from the group consisting of formulae A to E as below: 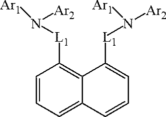 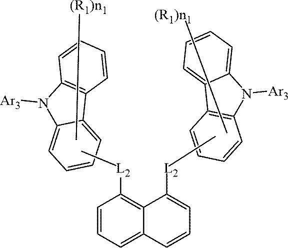 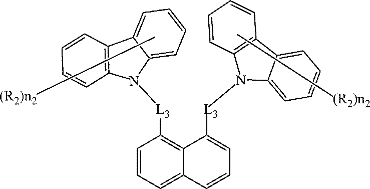 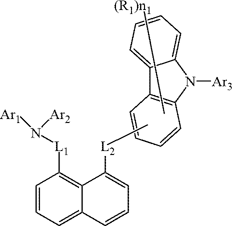 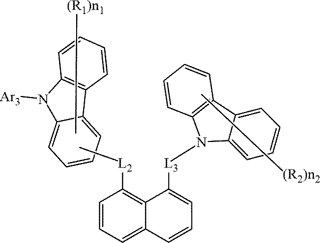
R₁ and R₂ are the same as or different from each other, and are each independently selected from deuterium, fluorine, cyano, an alkyl with 1 to 4 carbon atoms, a haloalkyl with 1 to 4 carbon atoms, a cycloalkyl with 5 to 10 carbon atoms, a group B, a heteroaryl with 3 to 18 carbon atoms, an alkoxy with 1 to 4 carbon atoms, an alkylthio with 1 to 4 carbon atoms, or a trialkylsilyl with 3 to 7 carbon atoms; and the group B is selected from a substituted or unsubstituted aryl with a total number of carbon atoms being 6 to 15, and substituents in the aryl are each independently selected from deuterium, fluorine, cyano, an alkyl with 1 to 4 carbon atoms, a fluoroalkyl with 1 to 4 carbon atoms, or a trialkylsilyl with 3 to 7 carbon atoms.

3. The nitrogen-containing compound of claim 1, wherein Ar₁, Ar2 and Ar₃ are the same as or different from each other, and are each independently selected from a substituted or unsubstituted aryl with a total number of carbon atoms being 6 to 25, or a substituted or unsubstituted heteroaryl with a total number of carbon atoms being 5 to 25 .

4. The nitrogen-containing compound of claim 1, wherein Ar₁, Ar₂ and Ar₃ are the same as or different from each other, and are each independently selected from the group consisting of groups represented by formulae i-1 to i-15: 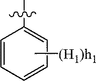 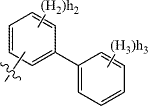 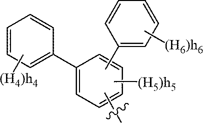 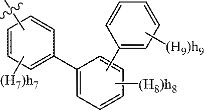 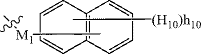 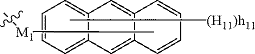 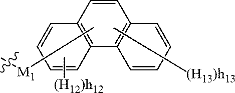 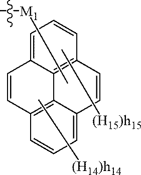 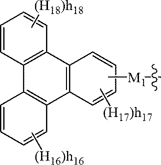 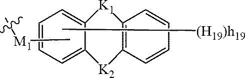 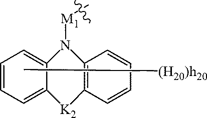 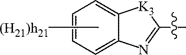 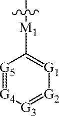 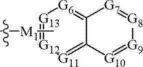 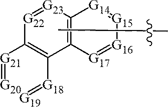
wherein Mi is selected from single bond or 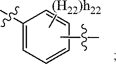
G₁ to G₅ are each independently selected from N or C(F₁), and at least one of G₁ to G₅ is selected from N; and when two or more of G₁ to G₅ are selected from C(F₁), any two F₁ are the same or different;
G₆ to G₁₃ are each independently selected from N or C(F₂), and at least one of G₆ to G₁₃ is selected from N; and when two or more of G₆ to G₁₃ are selected from C(F₂), any two F₂ are the same or different;
G₁₄ to G₂₃ are each independently selected from N or C(F₃), and at least one of G₁₄ to G₂₃ is selected from N; and when two or more of G₁₄ to G₂₃ are selected from C(F₃), any two F₃ are the same or different;
H₁ is selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or an alkylthio with 1 to 10 carbon atoms;
H₂ to H₉, and H₂₂ are each independently selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a heteroaryl with 3 to 18 carbon atoms;
H₁₀ to H₂₁, and F₁ to F₄ are each independently selected from hydrogen, deuterium, fluorine, chlorine, bromine, cyano, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, an aryl with 6 to 18 carbon atoms, or a heteroaryl with 3 to 18 carbon atoms;
h₁ to h₂₂ are represented by hₖ, H₁ to H₂₂ are represented by Hₖ, k is a variable, and represents any integer from 1 to 22, and hₖ represents the number of a substituent Hₖ; when k is selected from 5 or 17, hₖ is selected from 1, 2 or 3; when k is selected from 2, 7, 8, 12, 15, 16, 18, 21 or 22, hₖ is selected from 1, 2, 3 or 4; when k is selected from 1, 3, 4, 6, 9 or 14, hₖ is selected from 1, 2, 3, 4 or 5; when k is 13, hₖ is selected from 1, 2, 3, 4, 5, or 6; when k is selected from 10 or 19, hₖ is selected from 1, 2, 3, 4, 5, 6 or 7; when k is 20, hₖ is selected from 1, 2, 3, 4, 5, 6, 7, or 8; when k is 11, hₖ is selected from 1, 2, 3, 4, 5, 6, 7, 8 or 9; and when hₖ is greater than 1, any two Hₖ are the same or different;
K₁ is selected from O, S, Se, N(H₂₃), C(H₂₄H₂₅), or Si(H₂₄H₂₅); wherein H₂₃, H₂₄, and H₂₅ are each independently selected from an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₄ and the H₂₅ are connected to each other to form a 5- to 13-membered saturated or unsaturated ring together with the atom to which they are commonly connected;
K₂ is selected from single bond, O, S, Se, N(H₂₆), C(H₂₇H₂₈), or Si(H₂₇H₂₈); wherein H₂₆, H₂₇, and H₂₈ are each independently selected from an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₇ and the H₂₈ are connected to each other to form a 5- to 13-membered saturated or unsaturated ring together with the atom to which they are commonly connected; and
K₃ is selected from O or S.

5. The nitrogen-containing compound of claim 1, wherein Ar₁, Ar2 and Ar₃ are the same as or different from each other, and are each independently selected from a substituted or unsubstituted group Z, wherein the unsubstituted group Z is selected from the group consisting of: 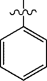 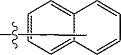 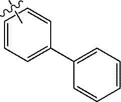 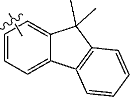 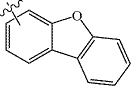 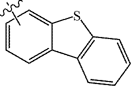 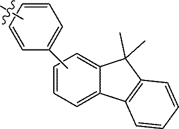 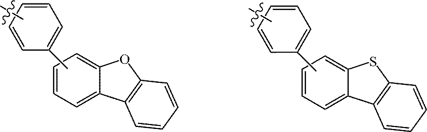 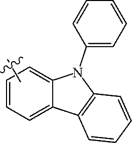 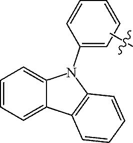 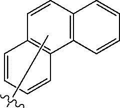 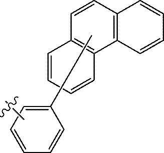 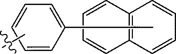 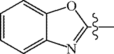 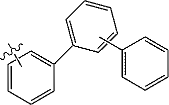 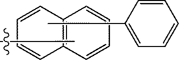 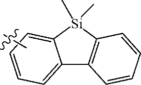 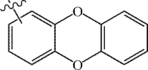 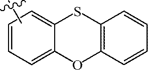 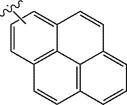 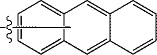 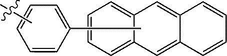 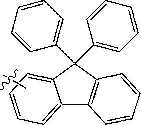 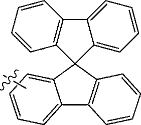 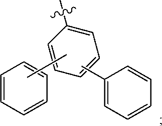
and the substituted group Z has one or two or more substituents, and the substituents are respectively and independently selected from deuterium, cyano, fluorine, an alkyl with 1 to 4 carbon atoms, a cycloalkyl with 5 to 10 carbon atoms, a haloalkyl with 1 to 4 carbon atoms, or a trialkylsilyl with 3 to 7 carbon atoms; and when the number of the substituents is greater than 1, the substituents are the same or different.

6. The nitrogen-containing compound of claim 1, wherein Ar₁, Ar₂ and Ar₃ are the same as or different from each other, and are each independently selected from the group consisting of: 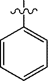 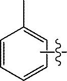 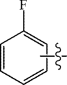 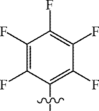 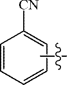 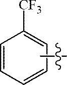 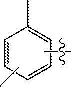 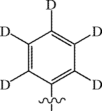 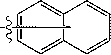 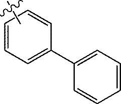 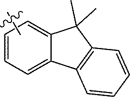 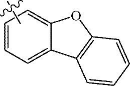 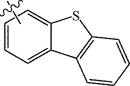 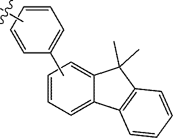 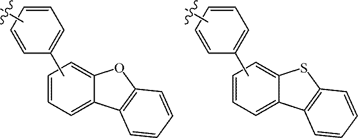 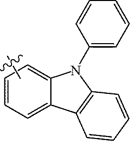 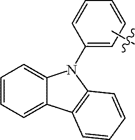 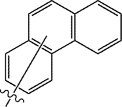 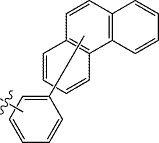 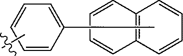 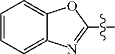 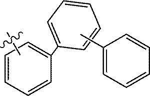 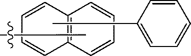 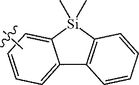 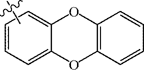 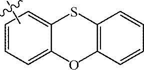 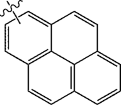 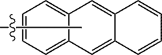 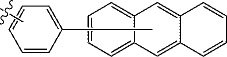 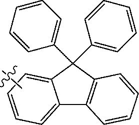 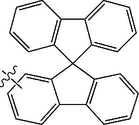 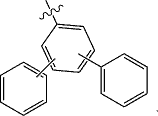
.

7. The nitrogen-containing compound of claim 1, wherein L₁ and L₃ are the same as or different from each other, and are each independently selected from a substituted or unsubstituted arylene with a total number of carbon atoms being 6 to 12, or a substituted or unsubstituted heteroarylene with a total number of carbon atoms being 5 to 12; and L₂ is selected from single bond, a substituted or unsubstituted arylene with a total number of carbon atoms being 6 to 12, or a substituted or unsubstituted heteroarylene with a total number of carbon atoms being 5 to 12 .

8. The nitrogen-containing compound of claim 1, wherein L₁, L₂ and L₃ are the same or different, and are each independently selected from the group consisting of groups represented by formulae j-1 to j-4 below: 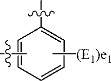 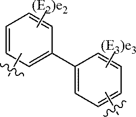 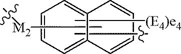 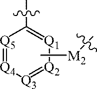
wherein M₂ is selected from single bond or 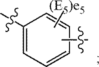
Q₁ to Q₅ are each independently selected from N or C(F₄), and at least one of Q₁ to Q₅ is selected from N; and when two or more of Q₁ to Q₅ are selected from C(F₄), any two F₄ are the same or different;
E₁ to E₅, and F₄ are each independently selected from hydrogen, deuterium, fluorine, chlorine, bromine, cyano, a heteroaryl with 3 to 10 carbon atoms, an aryl with 6 to 12 carbon atoms, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or an alkylthio with 1 to 10 carbon atoms; and
E₁ to E₅ are represented by Eᵣ, and e₁ to e₅ are represented by eᵣ, wherein r represents a variable and is selected from any integer from 1 to 5; when r is selected from 1, 2, 3 or 5, eᵣ is selected from 1, 2, 3 or 4; when r is 4, eᵣ is selected from 1, 2, 3, 4, 5, or 6; and when eᵣ is greater than 1, any two Eᵣ are the same or different.

9. The nitrogen-containing compound of claim 1, wherein L₁, L₂ and L₃ are the same as or different from each other, and are each independently selected from a substituted or unsubstituted group V, wherein the unsubstituted group V is selected from the group consisting of: 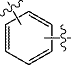 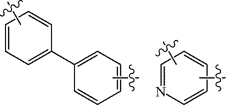 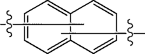 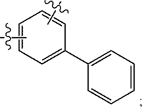
and the substituted group V has one or two or more substituents, and the substituents are respectively and independently selected from deuterium, cyano, fluorine, an alkyl with 1 to 4 carbon atoms, a cycloalkyl with 5 to 10 carbon atoms, a haloalkyl with 1 to 4 carbon atoms, or a trialkylsilyl with 3 to 7 carbon atoms, and when the number of the substituents is greater than 1, the substituents are the same or different.

10. The nitrogen-containing compound of claim 2, wherein the nitrogen-containing compound has a structure as shown in the formula A or the formula D; Ar₁, Ar₂ and Ar₃ are the same as or different from each other, and are each independently selected from a substituted or unsubstituted aryl with a total number of carbon atoms being 6 to 25, or a substituted or unsubstituted heteroaryl with a total number of carbon atoms being 5 to 20; or
the nitrogen-containing compound has a structure as shown in the formula B, the formula C, or the formula E; Ar₃ is selected from a substituted or unsubstituted aryl with a total number of carbon atoms being 6 to 20, or a substituted or unsubstituted heteroaryl with a total number of carbon atoms being 5 to 20.

11. The nitrogen-containing compound of claim 2, wherein the nitrogen-containing compound has a structure as shown in the formula A, wherein Ar₁ is selected from a substituted or unsubstituted aryl with 6 to 20 ring-forming carbon atoms, Ar₂ is selected from a substituted or unsubstituted aryl with 10 to 25 ring-forming carbon atoms, or a substituted or unsubstituted heteroaryl with 7 to 20 ring-forming carbon atoms; or
the nitrogen-containing compound has a structure as shown in the formula C, wherein at least one L₃ in the two L₃ is selected from a substituted or unsubstituted arylene with 10 to 20 ring-forming carbon atoms.

12. The nitrogen-containing compound of claim 1, wherein the nitrogen-containing compound is selected from the group consisting of the following compounds:

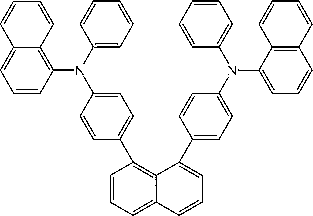 [SMILES: c1ccc(N(c2ccc(-c3cccc4cccc(-c5ccc(N(c6ccccc6)c6cccc7ccccc67)cc5)c34)cc2)c2cccc3ccccc23)cc1]

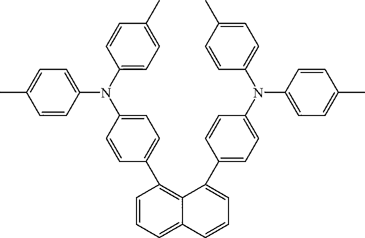

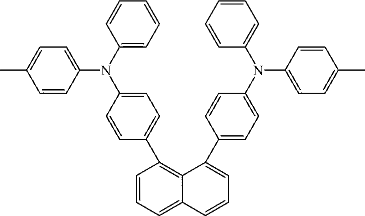

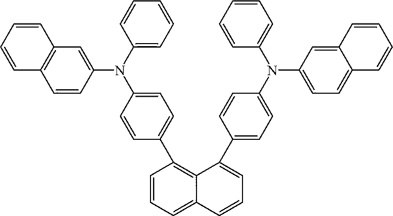 [SMILES: c1ccc(N(c2ccc(-c3cccc4cccc(-c5ccc(N(c6ccccc6)c6ccc7ccccc7c6)cc5)c34)cc2)c2ccc3ccccc3c2)cc1]

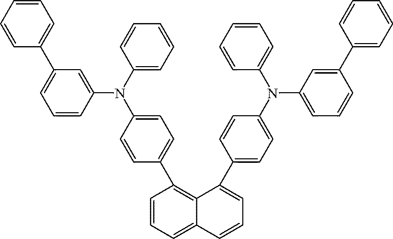 [SMILES: c1ccc(-c2cccc(N(c3ccccc3)c3ccc(-c4cccc5cccc(-c6ccc(N(c7ccccc7)c7cccc(-c8ccccc8)c7)cc6)c45)cc3)c2)cc1]

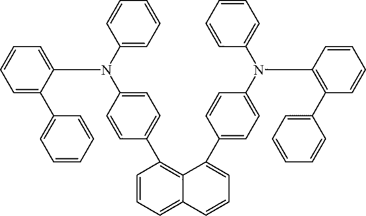 [SMILES: c1ccc(-c2ccccc2N(c2ccccc2)c2ccc(-c3cccc4cccc(-c5ccc(N(c6ccccc6)c6ccccc6-c6ccccc6)cc5)c34)cc2)cc1]

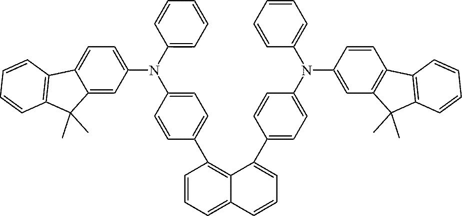 [SMILES: CC1(C)c2ccccc2-c2ccc(N(c3ccccc3)c3ccc(-c4cccc5cccc(-c6ccc(N(c7ccccc7)c7ccc8c(c7)C(C)(C)c7ccccc7-8)cc6)c45)cc3)cc21]

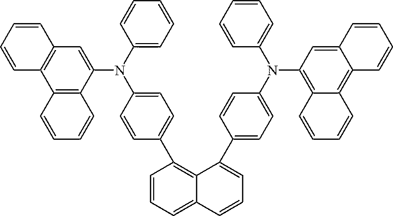 [SMILES: c1ccc(N(c2ccc(-c3cccc4cccc(-c5ccc(N(c6ccccc6)c6cc7ccccc7c7ccccc67)cc5)c34)cc2)c2cc3ccccc3c3ccccc23)cc1]

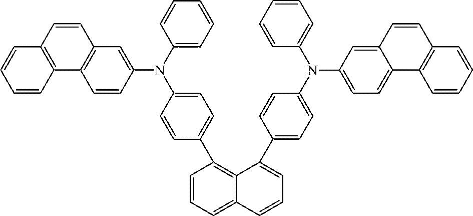 [SMILES: c1ccc(N(c2ccc(-c3cccc4cccc(-c5ccc(N(c6ccccc6)c6ccc7c(ccc8ccccc87)c6)cc5)c34)cc2)c2ccc3c(ccc4ccccc43)c2)cc1]

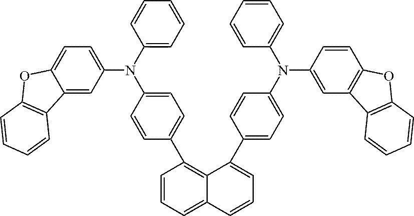 [SMILES: c1ccc(N(c2ccc(-c3cccc4cccc(-c5ccc(N(c6ccccc6)c6ccc7oc8ccccc8c7c6)cc5)c34)cc2)c2ccc3oc4ccccc4c3c2)cc1]

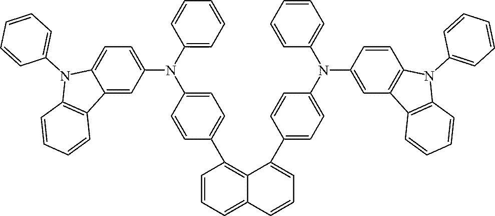 [SMILES: c1ccc(N(c2ccc(-c3cccc4cccc(-c5ccc(N(c6ccccc6)c6ccc7c(c6)c6ccccc6n7-c6ccccc6)cc5)c34)cc2)c2ccc3c(c2)c2ccccc2n3-c2ccccc2)cc1]

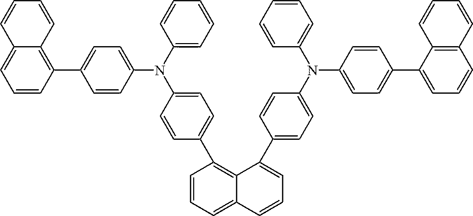 [SMILES: c1ccc(N(c2ccc(-c3cccc4ccccc34)cc2)c2ccc(-c3cccc4cccc(-c5ccc(N(c6ccccc6)c6ccc(-c7cccc8ccccc78)cc6)cc5)c34)cc2)cc1]

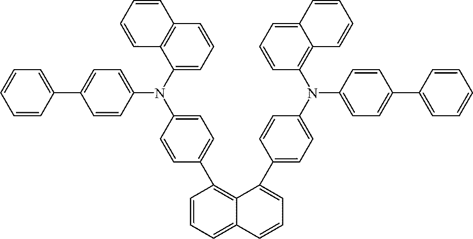 [SMILES: c1ccc(-c2ccc(N(c3ccc(-c4cccc5cccc(-c6ccc(N(c7ccc(-c8ccccc8)cc7)c7cccc8ccccc78)cc6)c45)cc3)c3cccc4ccccc34)cc2)cc1]

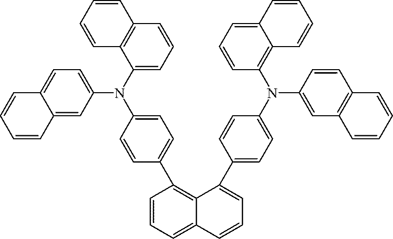 [SMILES: c1ccc2cc(N(c3ccc(-c4cccc5cccc(-c6ccc(N(c7ccc8ccccc8c7)c7cccc8ccccc78)cc6)c45)cc3)c3cccc4ccccc34)ccc2c1]

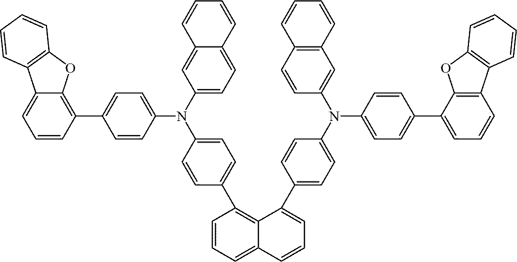 [SMILES: c1ccc2cc(N(c3ccc(-c4cccc5c4oc4ccccc45)cc3)c3ccc(-c4cccc5cccc(-c6ccc(N(c7ccc(-c8cccc9c8oc8ccccc89)cc7)c7ccc8ccccc8c7)cc6)c45)cc3)ccc2c1]

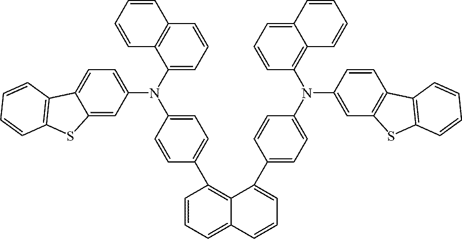 [SMILES: c1ccc2c(N(c3ccc(-c4cccc5cccc(-c6ccc(N(c7ccc8c(c7)sc7ccccc78)c7cccc8ccccc78)cc6)c45)cc3)c3ccc4c(c3)sc3ccccc34)cccc2c1]

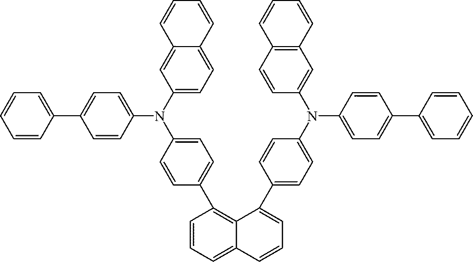 [SMILES: c1ccc(-c2ccc(N(c3ccc(-c4cccc5cccc(-c6ccc(N(c7ccc(-c8ccccc8)cc7)c7ccc8ccccc8c7)cc6)c45)cc3)c3ccc4ccccc4c3)cc2)cc1]

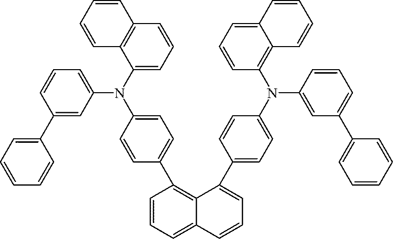 [SMILES: c1ccc(-c2cccc(N(c3ccc(-c4cccc5cccc(-c6ccc(N(c7cccc(-c8ccccc8)c7)c7cccc8ccccc78)cc6)c45)cc3)c3cccc4ccccc34)c2)cc1]

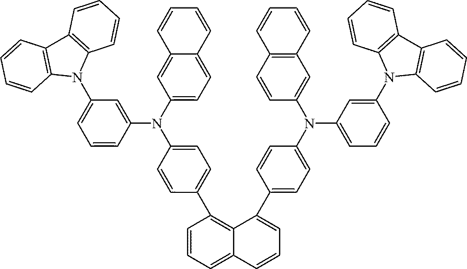 [SMILES: c1cc(N(c2ccc(-c3cccc4cccc(-c5ccc(N(c6cccc(-n7c8ccccc8c8ccccc87)c6)c6ccc7ccccc7c6)cc5)c34)cc2)c2ccc3ccccc3c2)cc(-n2c3ccccc3c3ccccc32)c1]

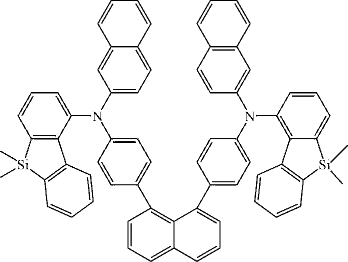

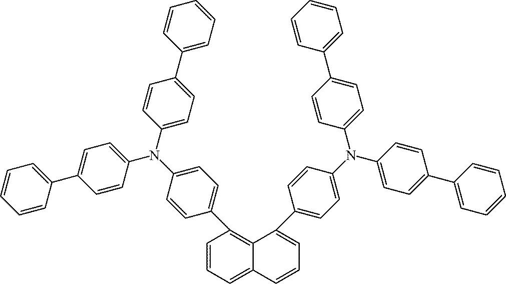 [SMILES: c1ccc(-c2ccc(N(c3ccc(-c4ccccc4)cc3)c3ccc(-c4cccc5cccc(-c6ccc(N(c7ccc(-c8ccccc8)cc7)c7ccc(-c8ccccc8)cc7)cc6)c45)cc3)cc2)cc1]

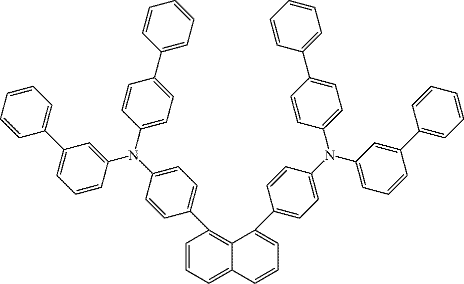 [SMILES: c1ccc(-c2ccc(N(c3ccc(-c4cccc5cccc(-c6ccc(N(c7ccc(-c8ccccc8)cc7)c7cccc(-c8ccccc8)c7)cc6)c45)cc3)c3cccc(-c4ccccc4)c3)cc2)cc1]

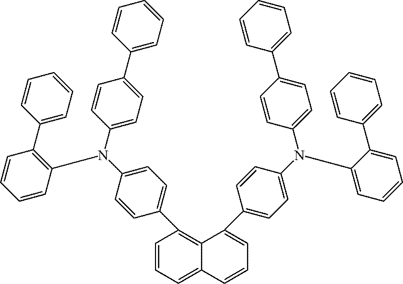 [SMILES: c1ccc(-c2ccc(N(c3ccc(-c4cccc5cccc(-c6ccc(N(c7ccc(-c8ccccc8)cc7)c7ccccc7-c7ccccc7)cc6)c45)cc3)c3ccccc3-c3ccccc3)cc2)cc1]

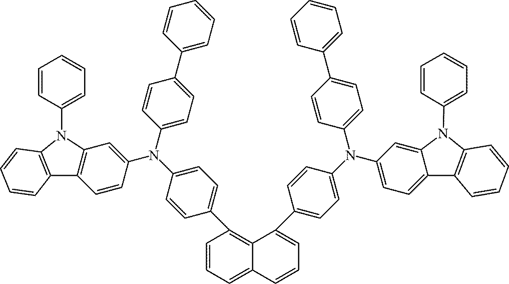 [SMILES: c1ccc(-c2ccc(N(c3ccc(-c4cccc5cccc(-c6ccc(N(c7ccc(-c8ccccc8)cc7)c7ccc8c9ccccc9n(-c9ccccc9)c8c7)cc6)c45)cc3)c3ccc4c5ccccc5n(-c5ccccc5)c4c3)cc2)cc1]

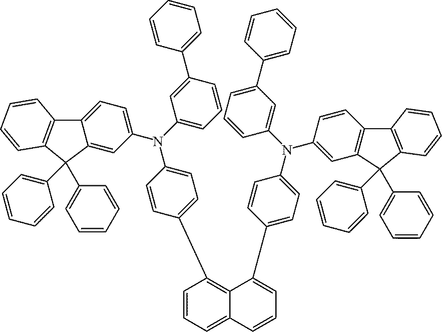 [SMILES: c1ccc(-c2cccc(N(c3ccc(-c4cccc5cccc(-c6ccc(N(c7cccc(-c8ccccc8)c7)c7ccc8c(c7)C(c7ccccc7)(c7ccccc7)c7ccccc7-8)cc6)c45)cc3)c3ccc4c(c3)C(c3ccccc3)(c3ccccc3)c3ccccc3-4)c2)cc1]

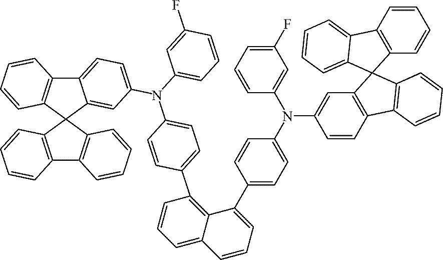 [SMILES: Fc1cccc(N(c2ccc(-c3cccc4cccc(-c5ccc(N(c6cccc(F)c6)c6ccc7c(c6)C6(c8ccccc8-c8ccccc86)c6ccccc6-7)cc5)c34)cc2)c2ccc3c(c2)C2(c4ccccc4-c4ccccc42)c2ccccc2-3)c1]

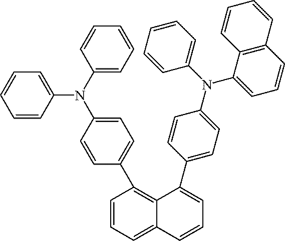 [SMILES: c1ccc(N(c2ccccc2)c2ccc(-c3cccc4cccc(-c5ccc(N(c6ccccc6)c6cccc7ccccc67)cc5)c34)cc2)cc1]

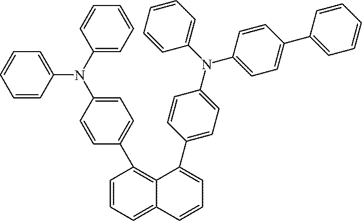 [SMILES: c1ccc(-c2ccc(N(c3ccccc3)c3ccc(-c4cccc5cccc(-c6ccc(N(c7ccccc7)c7ccccc7)cc6)c45)cc3)cc2)cc1]

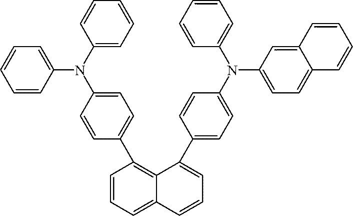 [SMILES: c1ccc(N(c2ccccc2)c2ccc(-c3cccc4cccc(-c5ccc(N(c6ccccc6)c6ccc7ccccc7c6)cc5)c34)cc2)cc1]

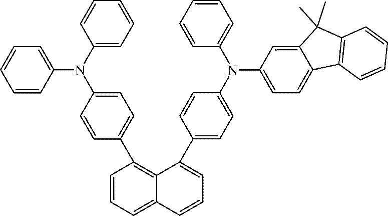 [SMILES: CC1(C)c2ccccc2-c2ccc(N(c3ccccc3)c3ccc(-c4cccc5cccc(-c6ccc(N(c7ccccc7)c7ccccc7)cc6)c45)cc3)cc21]

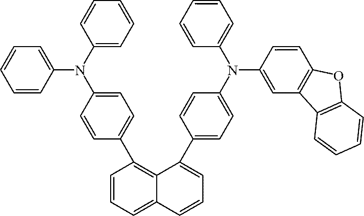 [SMILES: c1ccc(N(c2ccccc2)c2ccc(-c3cccc4cccc(-c5ccc(N(c6ccccc6)c6ccc7oc8ccccc8c7c6)cc5)c34)cc2)cc1]

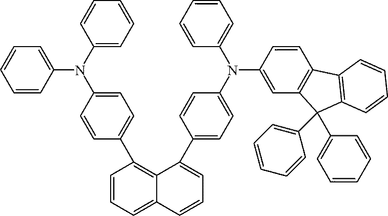 [SMILES: c1ccc(N(c2ccccc2)c2ccc(-c3cccc4cccc(-c5ccc(N(c6ccccc6)c6ccc7c(c6)C(c6ccccc6)(c6ccccc6)c6ccccc6-7)cc5)c34)cc2)cc1]

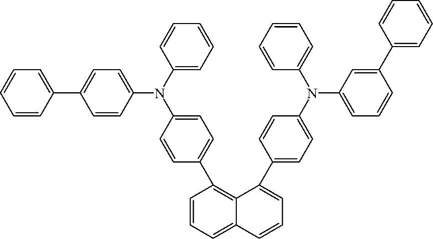 [SMILES: c1ccc(-c2ccc(N(c3ccccc3)c3ccc(-c4cccc5cccc(-c6ccc(N(c7ccccc7)c7cccc(-c8ccccc8)c7)cc6)c45)cc3)cc2)cc1]

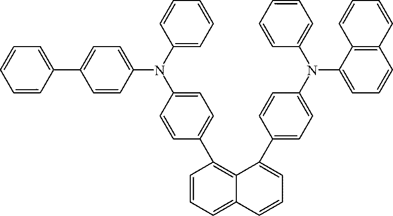 [SMILES: c1ccc(-c2ccc(N(c3ccccc3)c3ccc(-c4cccc5cccc(-c6ccc(N(c7ccccc7)c7cccc8ccccc78)cc6)c45)cc3)cc2)cc1]

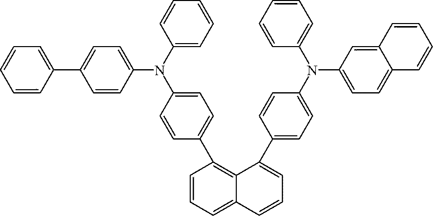 [SMILES: c1ccc(-c2ccc(N(c3ccccc3)c3ccc(-c4cccc5cccc(-c6ccc(N(c7ccccc7)c7ccc8ccccc8c7)cc6)c45)cc3)cc2)cc1]

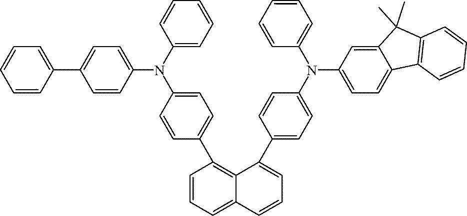 [SMILES: CC1(C)c2ccccc2-c2ccc(N(c3ccccc3)c3ccc(-c4cccc5cccc(-c6ccc(N(c7ccccc7)c7ccc(-c8ccccc8)cc7)cc6)c45)cc3)cc21]

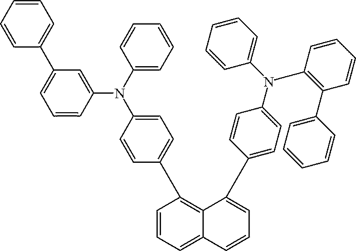 [SMILES: c1ccc(-c2cccc(N(c3ccccc3)c3ccc(-c4cccc5cccc(-c6ccc(N(c7ccccc7)c7ccccc7-c7ccccc7)cc6)c45)cc3)c2)cc1]

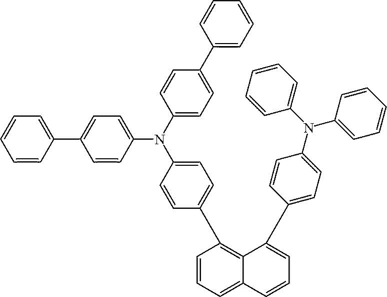 [SMILES: c1ccc(-c2ccc(N(c3ccc(-c4ccccc4)cc3)c3ccc(-c4cccc5cccc(-c6ccc(N(c7ccccc7)c7ccccc7)cc6)c45)cc3)cc2)cc1]

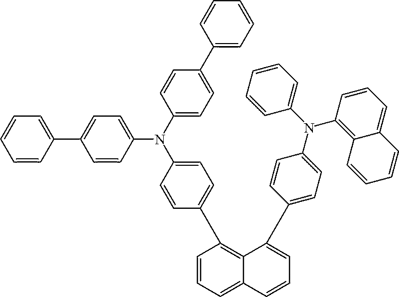 [SMILES: c1ccc(-c2ccc(N(c3ccc(-c4ccccc4)cc3)c3ccc(-c4cccc5cccc(-c6ccc(N(c7ccccc7)c7cccc8ccccc78)cc6)c45)cc3)cc2)cc1]

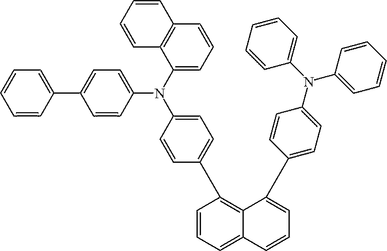 [SMILES: c1ccc(-c2ccc(N(c3ccc(-c4cccc5cccc(-c6ccc(N(c7ccccc7)c7ccccc7)cc6)c45)cc3)c3cccc4ccccc34)cc2)cc1]

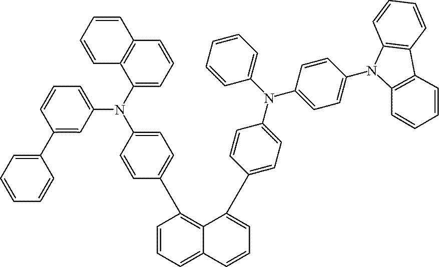 [SMILES: c1ccc(-c2cccc(N(c3ccc(-c4cccc5cccc(-c6ccc(N(c7ccccc7)c7ccc(-n8c9ccccc9c9ccccc98)cc7)cc6)c45)cc3)c3cccc4ccccc34)c2)cc1]

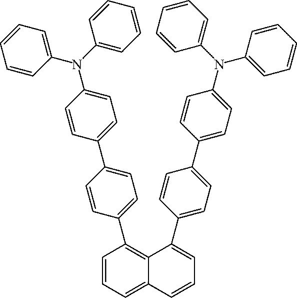 [SMILES: c1ccc(N(c2ccccc2)c2ccc(-c3ccc(-c4cccc5cccc(-c6ccc(-c7ccc(N(c8ccccc8)c8ccccc8)cc7)cc6)c45)cc3)cc2)cc1]

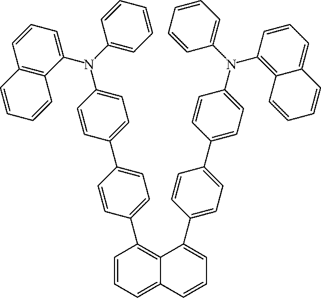 [SMILES: c1ccc(N(c2ccc(-c3ccc(-c4cccc5cccc(-c6ccc(-c7ccc(N(c8ccccc8)c8cccc9ccccc89)cc7)cc6)c45)cc3)cc2)c2cccc3ccccc23)cc1]

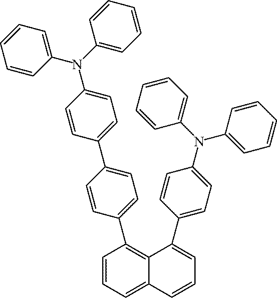 [SMILES: c1ccc(N(c2ccccc2)c2ccc(-c3ccc(-c4cccc5cccc(-c6ccc(N(c7ccccc7)c7ccccc7)cc6)c45)cc3)cc2)cc1]

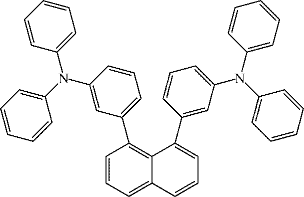 [SMILES: c1ccc(N(c2ccccc2)c2cccc(-c3cccc4cccc(-c5cccc(N(c6ccccc6)c6ccccc6)c5)c34)c2)cc1]

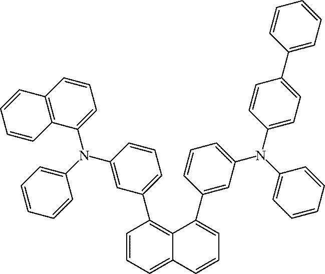 [SMILES: c1ccc(-c2ccc(N(c3ccccc3)c3cccc(-c4cccc5cccc(-c6cccc(N(c7ccccc7)c7cccc8ccccc78)c6)c45)c3)cc2)cc1]

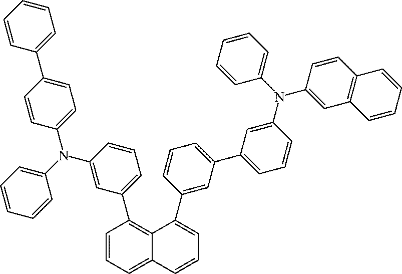 [SMILES: c1ccc(-c2ccc(N(c3ccccc3)c3cccc(-c4cccc5cccc(-c6cccc(-c7cccc(N(c8ccccc8)c8ccc9ccccc9c8)c7)c6)c45)c3)cc2)cc1]

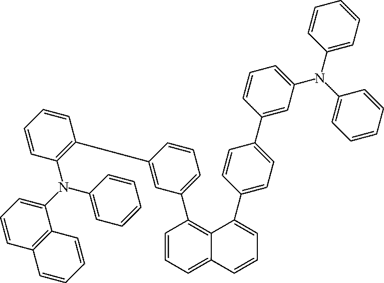 [SMILES: c1ccc(N(c2ccccc2)c2cccc(-c3ccc(-c4cccc5cccc(-c6cccc(-c7ccccc7N(c7ccccc7)c7cccc8ccccc78)c6)c45)cc3)c2)cc1]

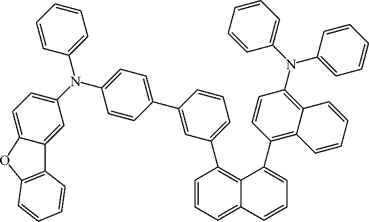 [SMILES: c1ccc(N(c2ccc(-c3cccc(-c4cccc5cccc(-c6ccc(N(c7ccccc7)c7ccccc7)c7ccccc67)c45)c3)cc2)c2ccc3oc4ccccc4c3c2)cc1]

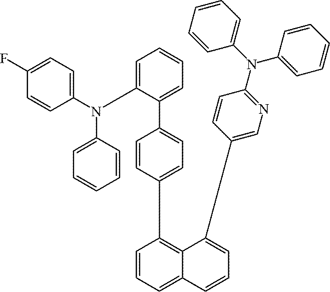 [SMILES: Fc1ccc(N(c2ccccc2)c2ccccc2-c2ccc(-c3cccc4cccc(-c5ccc(N(c6ccccc6)c6ccccc6)nc5)c34)cc2)cc1]

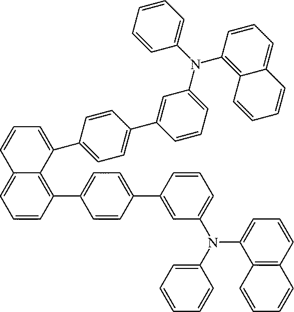 [SMILES: c1ccc(N(c2cccc(-c3ccc(-c4cccc5cccc(-c6ccc(-c7cccc(N(c8ccccc8)c8cccc9ccccc89)c7)cc6)c45)cc3)c2)c2cccc3ccccc23)cc1]

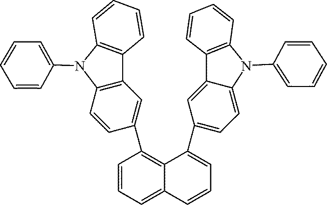 [SMILES: c1ccc(-n2c3ccccc3c3cc(-c4cccc5cccc(-c6ccc7c(c6)c6ccccc6n7-c6ccccc6)c45)ccc32)cc1]

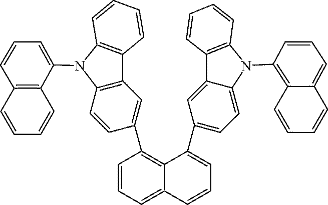 [SMILES: c1ccc2c(-n3c4ccccc4c4cc(-c5cccc6cccc(-c7ccc8c(c7)c7ccccc7n8-c7cccc8ccccc78)c56)ccc43)cccc2c1]

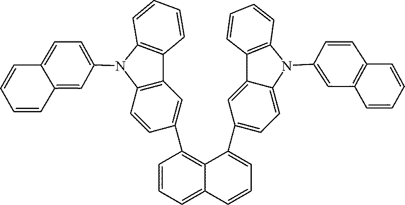 [SMILES: c1ccc2cc(-n3c4ccccc4c4cc(-c5cccc6cccc(-c7ccc8c(c7)c7ccccc7n8-c7ccc8ccccc8c7)c56)ccc43)ccc2c1]

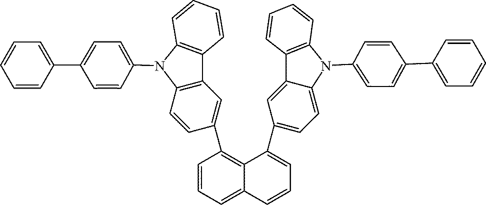 [SMILES: c1ccc(-c2ccc(-n3c4ccccc4c4cc(-c5cccc6cccc(-c7ccc8c(c7)c7ccccc7n8-c7ccc(-c8ccccc8)cc7)c56)ccc43)cc2)cc1]

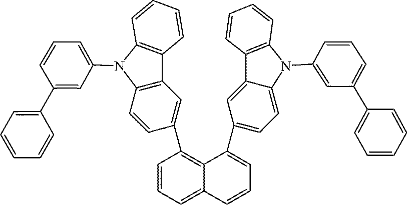 [SMILES: c1ccc(-c2cccc(-n3c4ccccc4c4cc(-c5cccc6cccc(-c7ccc8c(c7)c7ccccc7n8-c7cccc(-c8ccccc8)c7)c56)ccc43)c2)cc1]

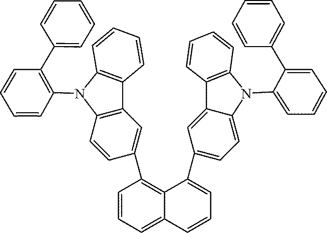 [SMILES: c1ccc(-c2ccccc2-n2c3ccccc3c3cc(-c4cccc5cccc(-c6ccc7c(c6)c6ccccc6n7-c6ccccc6-c6ccccc6)c45)ccc32)cc1]

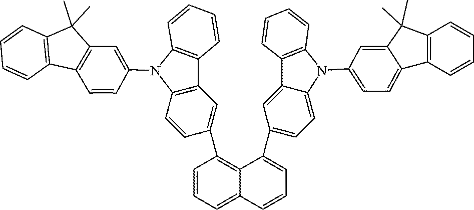 [SMILES: CC1(C)c2ccccc2-c2ccc(-n3c4ccccc4c4cc(-c5cccc6cccc(-c7ccc8c(c7)c7ccccc7n8-c7ccc8c(c7)C(C)(C)c7ccccc7-8)c56)ccc43)cc21]

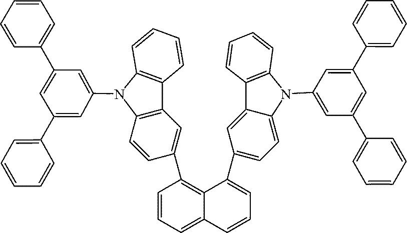 [SMILES: c1ccc(-c2cc(-c3ccccc3)cc(-n3c4ccccc4c4cc(-c5cccc6cccc(-c7ccc8c(c7)c7ccccc7n8-c7cc(-c8ccccc8)cc(-c8ccccc8)c7)c56)ccc43)c2)cc1]

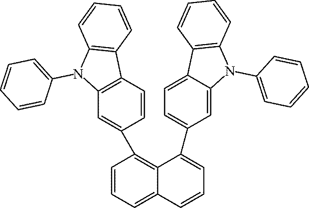 [SMILES: c1ccc(-n2c3ccccc3c3ccc(-c4cccc5cccc(-c6ccc7c8ccccc8n(-c8ccccc8)c7c6)c45)cc32)cc1]

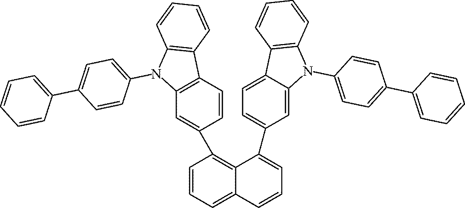 [SMILES: c1ccc(-c2ccc(-n3c4ccccc4c4ccc(-c5cccc6cccc(-c7ccc8c9ccccc9n(-c9ccc(-c%10ccccc%10)cc9)c8c7)c56)cc43)cc2)cc1]

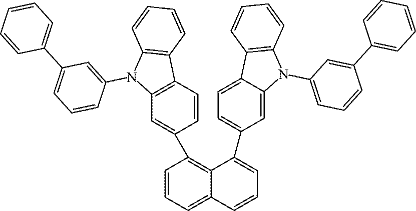 [SMILES: c1ccc(-c2cccc(-n3c4ccccc4c4ccc(-c5cccc6cccc(-c7ccc8c9ccccc9n(-c9cccc(-c%10ccccc%10)c9)c8c7)c56)cc43)c2)cc1]

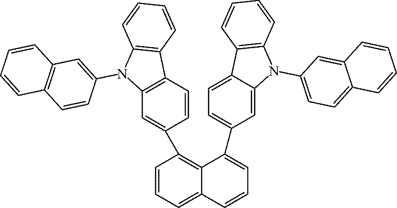 [SMILES: c1ccc2cc(-n3c4ccccc4c4ccc(-c5cccc6cccc(-c7ccc8c9ccccc9n(-c9ccc%10ccccc%10c9)c8c7)c56)cc43)ccc2c1]

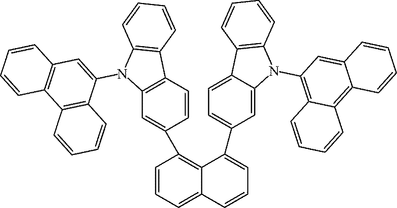 [SMILES: c1cc(-c2ccc3c4ccccc4n(-c4cc5ccccc5c5ccccc45)c3c2)c2c(-c3ccc4c5ccccc5n(-c5cc6ccccc6c6ccccc56)c4c3)cccc2c1]

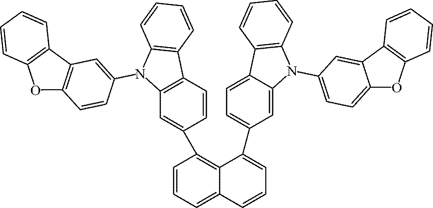 [SMILES: c1cc(-c2ccc3c4ccccc4n(-c4ccc5oc6ccccc6c5c4)c3c2)c2c(-c3ccc4c5ccccc5n(-c5ccc6oc7ccccc7c6c5)c4c3)cccc2c1]

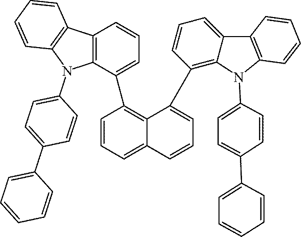 [SMILES: c1ccc(-c2ccc(-n3c4ccccc4c4cccc(-c5cccc6cccc(-c7cccc8c9ccccc9n(-c9ccc(-c%10ccccc%10)cc9)c78)c56)c43)cc2)cc1]

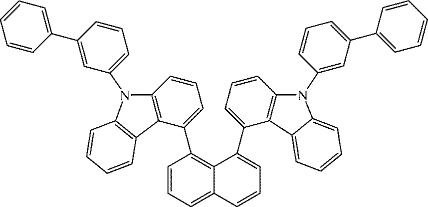 [SMILES: c1ccc(-c2cccc(-n3c4ccccc4c4c(-c5cccc6cccc(-c7cccc8c7c7ccccc7n8-c7cccc(-c8ccccc8)c7)c56)cccc43)c2)cc1]

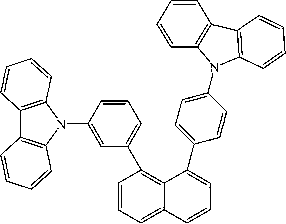 [SMILES: c1cc(-c2cccc3cccc(-c4ccc(-n5c6ccccc6c6ccccc65)cc4)c23)cc(-n2c3ccccc3c3ccccc32)c1]

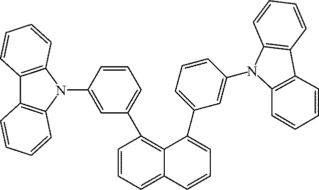 [SMILES: c1cc(-c2cccc3cccc(-c4cccc(-n5c6ccccc6c6ccccc65)c4)c23)cc(-n2c3ccccc3c3ccccc32)c1]

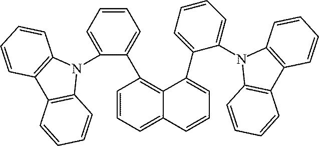 [SMILES: c1ccc(-n2c3ccccc3c3ccccc32)c(-c2cccc3cccc(-c4ccccc4-n4c5ccccc5c5ccccc54)c23)c1]

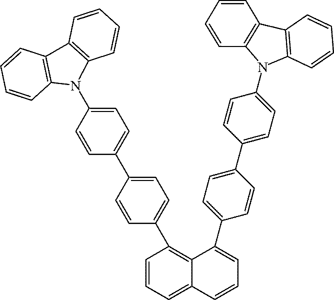 [SMILES: c1cc(-c2ccc(-c3ccc(-n4c5ccccc5c5ccccc54)cc3)cc2)c2c(-c3ccc(-c4ccc(-n5c6ccccc6c6ccccc65)cc4)cc3)cccc2c1]

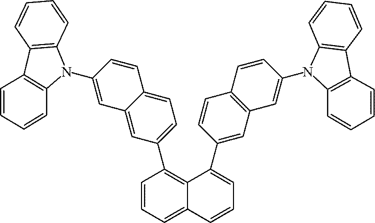 [SMILES: c1cc(-c2ccc3ccc(-n4c5ccccc5c5ccccc54)cc3c2)c2c(-c3ccc4ccc(-n5c6ccccc6c6ccccc65)cc4c3)cccc2c1]

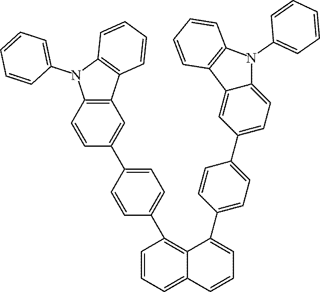 [SMILES: c1ccc(-n2c3ccccc3c3cc(-c4ccc(-c5cccc6cccc(-c7ccc(-c8ccc9c(c8)c8ccccc8n9-c8ccccc8)cc7)c56)cc4)ccc32)cc1]

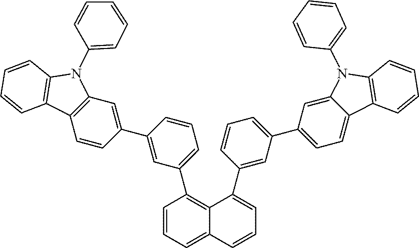 [SMILES: c1ccc(-n2c3ccccc3c3ccc(-c4cccc(-c5cccc6cccc(-c7cccc(-c8ccc9c%10ccccc%10n(-c%10ccccc%10)c9c8)c7)c56)c4)cc32)cc1]

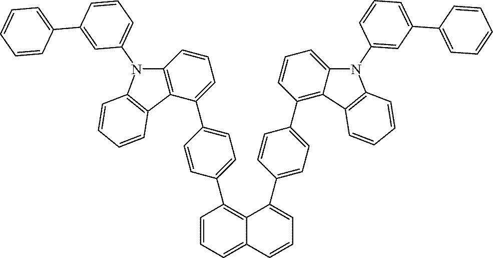 [SMILES: c1ccc(-c2cccc(-n3c4ccccc4c4c(-c5ccc(-c6cccc7cccc(-c8ccc(-c9cccc%10c9c9ccccc9n%10-c9cccc(-c%10ccccc%10)c9)cc8)c67)cc5)cccc43)c2)cc1]

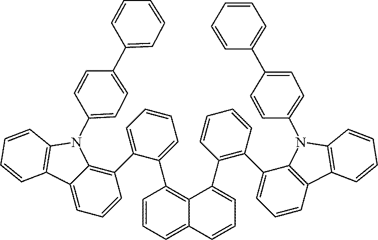 [SMILES: c1ccc(-c2ccc(-n3c4ccccc4c4cccc(-c5ccccc5-c5cccc6cccc(-c7ccccc7-c7cccc8c9ccccc9n(-c9ccc(-c%10ccccc%10)cc9)c78)c56)c43)cc2)cc1]

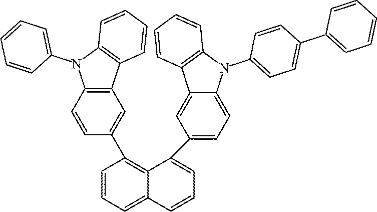 [SMILES: c1ccc(-c2ccc(-n3c4ccccc4c4cc(-c5cccc6cccc(-c7ccc8c(c7)c7ccccc7n8-c7ccccc7)c56)ccc43)cc2)cc1]

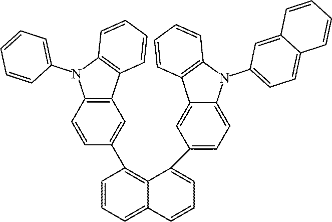 [SMILES: c1ccc(-n2c3ccccc3c3cc(-c4cccc5cccc(-c6ccc7c(c6)c6ccccc6n7-c6ccc7ccccc7c6)c45)ccc32)cc1]

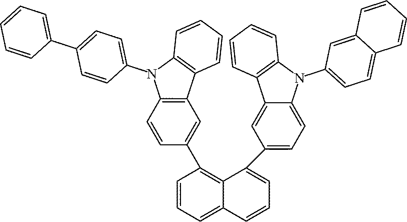 [SMILES: c1ccc(-c2ccc(-n3c4ccccc4c4cc(-c5cccc6cccc(-c7ccc8c(c7)c7ccccc7n8-c7ccc8ccccc8c7)c56)ccc43)cc2)cc1]

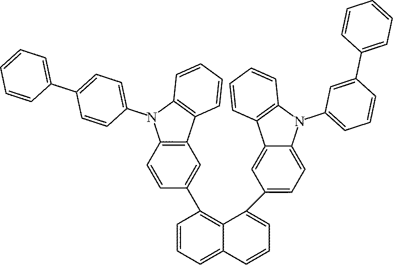 [SMILES: c1ccc(-c2ccc(-n3c4ccccc4c4cc(-c5cccc6cccc(-c7ccc8c(c7)c7ccccc7n8-c7cccc(-c8ccccc8)c7)c56)ccc43)cc2)cc1]

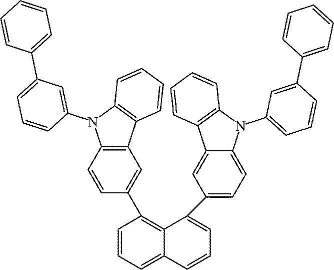 [SMILES: c1ccc(-c2cccc(-n3c4ccccc4c4cc(-c5cccc6cccc(-c7ccc8c(c7)c7ccccc7n8-c7cccc(-c8ccccc8)c7)c56)ccc43)c2)cc1]

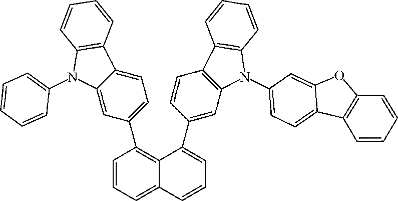 [SMILES: c1ccc(-n2c3ccccc3c3ccc(-c4cccc5cccc(-c6ccc7c8ccccc8n(-c8ccc9c(c8)oc8ccccc89)c7c6)c45)cc32)cc1]

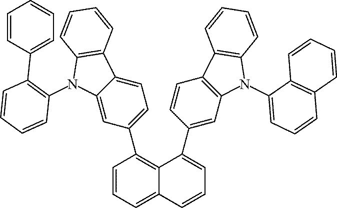 [SMILES: c1ccc(-c2ccccc2-n2c3ccccc3c3ccc(-c4cccc5cccc(-c6ccc7c8ccccc8n(-c8cccc9ccccc89)c7c6)c45)cc32)cc1]

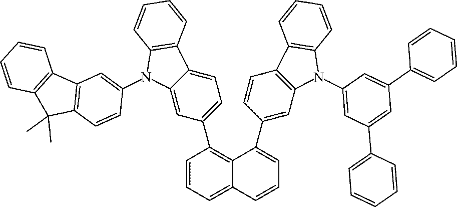 [SMILES: CC1(C)c2ccccc2-c2cc(-n3c4ccccc4c4ccc(-c5cccc6cccc(-c7ccc8c9ccccc9n(-c9cc(-c%10ccccc%10)cc(-c%10ccccc%10)c9)c8c7)c56)cc43)ccc21]

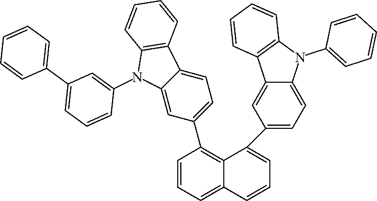 [SMILES: c1ccc(-c2cccc(-n3c4ccccc4c4ccc(-c5cccc6cccc(-c7ccc8c(c7)c7ccccc7n8-c7ccccc7)c56)cc43)c2)cc1]

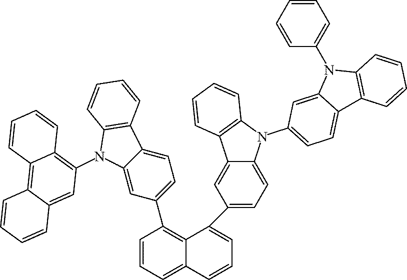 [SMILES: c1ccc(-n2c3ccccc3c3ccc(-n4c5ccccc5c5cc(-c6cccc7cccc(-c8ccc9c%10ccccc%10n(-c%10cc%11ccccc%11c%11ccccc%10%11)c9c8)c67)ccc54)cc32)cc1]

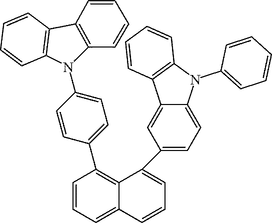 [SMILES: c1ccc(-n2c3ccccc3c3cc(-c4cccc5cccc(-c6ccc(-n7c8ccccc8c8ccccc87)cc6)c45)ccc32)cc1]

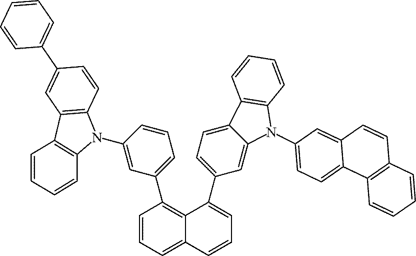 [SMILES: c1ccc(-c2ccc3c(c2)c2ccccc2n3-c2cccc(-c3cccc4cccc(-c5ccc6c7ccccc7n(-c7ccc8c(ccc9ccccc98)c7)c6c5)c34)c2)cc1]

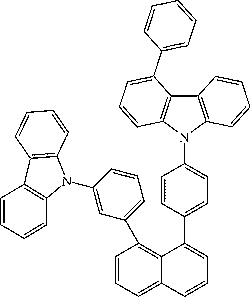 [SMILES: c1ccc(-c2cccc3c2c2ccccc2n3-c2ccc(-c3cccc4cccc(-c5cccc(-n6c7ccccc7c7ccccc76)c5)c34)cc2)cc1]

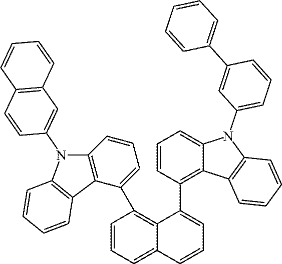 [SMILES: c1ccc(-c2cccc(-n3c4ccccc4c4c(-c5cccc6cccc(-c7cccc8c7c7ccccc7n8-c7ccc8ccccc8c7)c56)cccc43)c2)cc1]

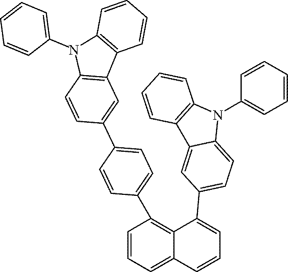 [SMILES: c1ccc(-n2c3ccccc3c3cc(-c4ccc(-c5cccc6cccc(-c7ccc8c(c7)c7ccccc7n8-c7ccccc7)c56)cc4)ccc32)cc1]

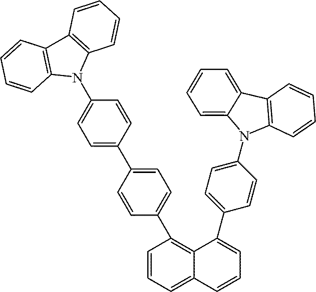 [SMILES: c1cc(-c2ccc(-c3ccc(-n4c5ccccc5c5ccccc54)cc3)cc2)c2c(-c3ccc(-n4c5ccccc5c5ccccc54)cc3)cccc2c1]

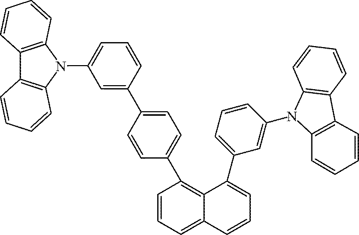 [SMILES: c1cc(-c2ccc(-c3cccc4cccc(-c5cccc(-n6c7ccccc7c7ccccc76)c5)c34)cc2)cc(-n2c3ccccc3c3ccccc32)c1]

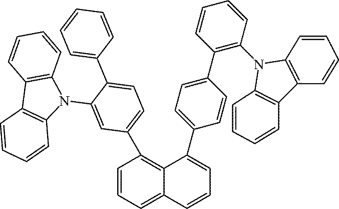 [SMILES: c1ccc(-c2ccc(-c3cccc4cccc(-c5ccc(-c6ccccc6-n6c7ccccc7c7ccccc76)cc5)c34)cc2-n2c3ccccc3c3ccccc32)cc1]

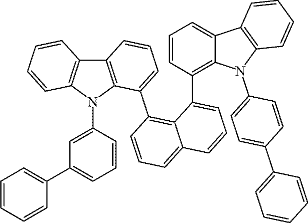 [SMILES: c1ccc(-c2ccc(-n3c4ccccc4c4cccc(-c5cccc6cccc(-c7cccc8c9ccccc9n(-c9cccc(-c%10ccccc%10)c9)c78)c56)c43)cc2)cc1]

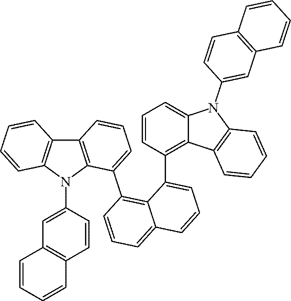 [SMILES: c1ccc2cc(-n3c4ccccc4c4c(-c5cccc6cccc(-c7cccc8c9ccccc9n(-c9ccc%10ccccc%10c9)c78)c56)cccc43)ccc2c1]

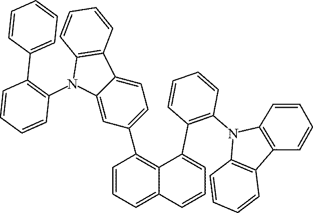 [SMILES: c1ccc(-c2ccccc2-n2c3ccccc3c3ccc(-c4cccc5cccc(-c6ccccc6-n6c7ccccc7c7ccccc76)c45)cc32)cc1]

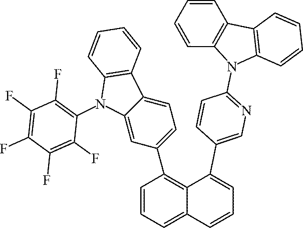 [SMILES: Fc1c(F)c(F)c(-n2c3ccccc3c3ccc(-c4cccc5cccc(-c6ccc(-n7c8ccccc8c8ccccc87)nc6)c45)cc32)c(F)c1F]

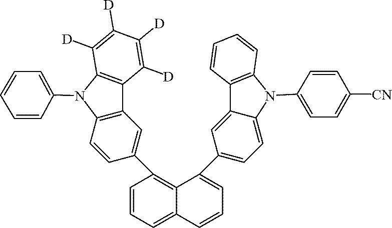 [SMILES: [2H]c1c([2H])c([2H])c2c(c1[2H])c1cc(-c3cccc4cccc(-c5ccc6c(c5)c5ccccc5n6-c5ccc(C#N)cc5)c34)ccc1n2-c1ccccc1]

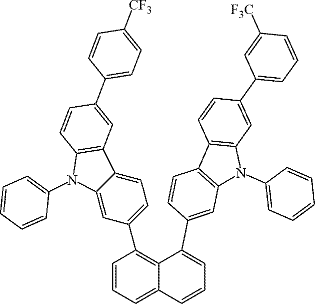 [SMILES: FC(F)(F)c1ccc(-c2ccc3c(c2)c2ccc(-c4cccc5cccc(-c6ccc7c8ccc(-c9cccc(C(F)(F)F)c9)cc8n(-c8ccccc8)c7c6)c45)cc2n3-c2ccccc2)cc1]

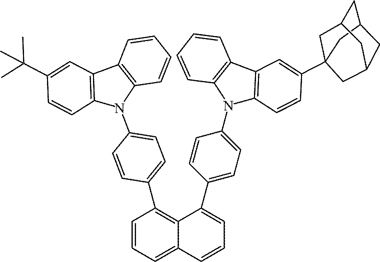 [SMILES: CC(C)(C)c1ccc2c(c1)c1ccccc1n2-c1ccc(-c2cccc3cccc(-c4ccc(-n5c6ccccc6c6cc(C78CC9CC(CC(C9)C7)C8)ccc65)cc4)c23)cc1]

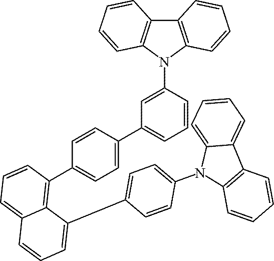 [SMILES: c1cc(-c2ccc(-c3cccc4cccc(-c5ccc(-n6c7ccccc7c7ccccc76)cc5)c34)cc2)cc(-n2c3ccccc3c3ccccc32)c1]

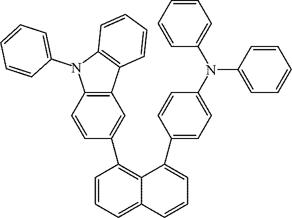 [SMILES: c1ccc(N(c2ccccc2)c2ccc(-c3cccc4cccc(-c5ccc6c(c5)c5ccccc5n6-c5ccccc5)c34)cc2)cc1]

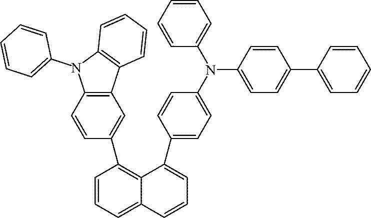 [SMILES: c1ccc(-c2ccc(N(c3ccccc3)c3ccc(-c4cccc5cccc(-c6ccc7c(c6)c6ccccc6n7-c6ccccc6)c45)cc3)cc2)cc1]

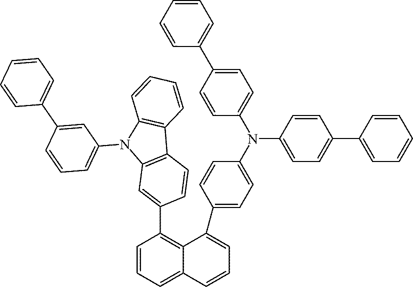 [SMILES: c1ccc(-c2ccc(N(c3ccc(-c4ccccc4)cc3)c3ccc(-c4cccc5cccc(-c6ccc7c8ccccc8n(-c8cccc(-c9ccccc9)c8)c7c6)c45)cc3)cc2)cc1]

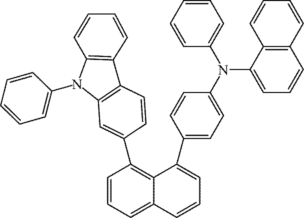 [SMILES: c1ccc(N(c2ccc(-c3cccc4cccc(-c5ccc6c7ccccc7n(-c7ccccc7)c6c5)c34)cc2)c2cccc3ccccc23)cc1]

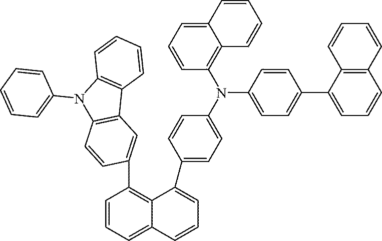 [SMILES: c1ccc(-n2c3ccccc3c3cc(-c4cccc5cccc(-c6ccc(N(c7ccc(-c8cccc9ccccc89)cc7)c7cccc8ccccc78)cc6)c45)ccc32)cc1]

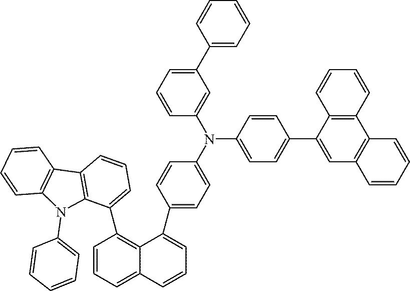 [SMILES: c1ccc(-c2cccc(N(c3ccc(-c4cc5ccccc5c5ccccc45)cc3)c3ccc(-c4cccc5cccc(-c6cccc7c8ccccc8n(-c8ccccc8)c67)c45)cc3)c2)cc1]

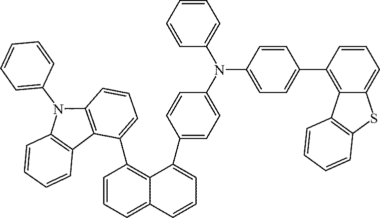 [SMILES: c1ccc(N(c2ccc(-c3cccc4cccc(-c5cccc6c5c5ccccc5n6-c5ccccc5)c34)cc2)c2ccc(-c3cccc4sc5ccccc5c34)cc2)cc1]

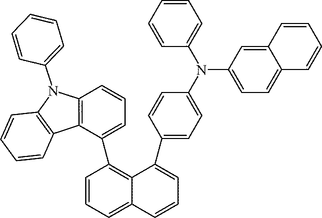 [SMILES: c1ccc(N(c2ccc(-c3cccc4cccc(-c5cccc6c5c5ccccc5n6-c5ccccc5)c34)cc2)c2ccc3ccccc3c2)cc1]

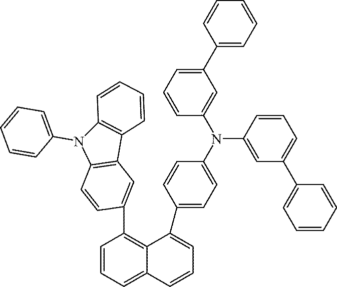 [SMILES: c1ccc(-c2cccc(N(c3ccc(-c4cccc5cccc(-c6ccc7c(c6)c6ccccc6n7-c6ccccc6)c45)cc3)c3cccc(-c4ccccc4)c3)c2)cc1]

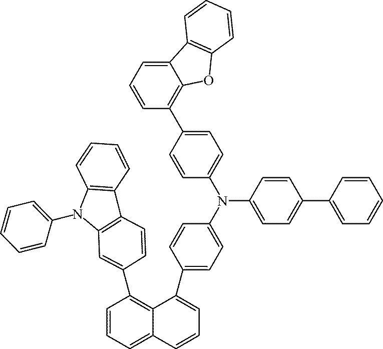 [SMILES: c1ccc(-c2ccc(N(c3ccc(-c4cccc5c4oc4ccccc45)cc3)c3ccc(-c4cccc5cccc(-c6ccc7c8ccccc8n(-c8ccccc8)c7c6)c45)cc3)cc2)cc1]

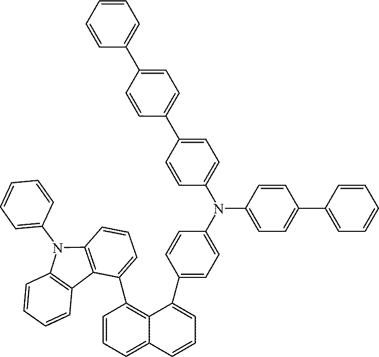 [SMILES: c1ccc(-c2ccc(-c3ccc(N(c4ccc(-c5ccccc5)cc4)c4ccc(-c5cccc6cccc(-c7cccc8c7c7ccccc7n8-c7ccccc7)c56)cc4)cc3)cc2)cc1]

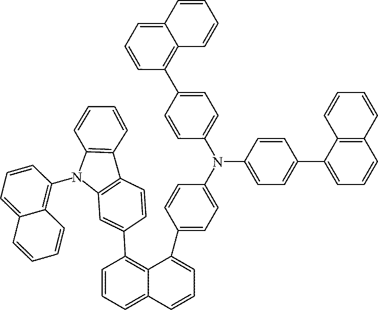 [SMILES: c1ccc2c(-c3ccc(N(c4ccc(-c5cccc6ccccc56)cc4)c4ccc(-c5cccc6cccc(-c7ccc8c9ccccc9n(-c9cccc%10ccccc9%10)c8c7)c56)cc4)cc3)cccc2c1]

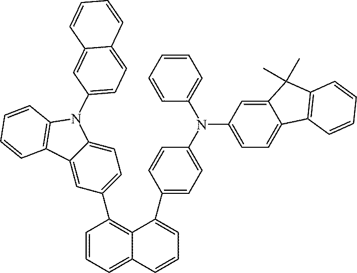 [SMILES: CC1(C)c2ccccc2-c2ccc(N(c3ccccc3)c3ccc(-c4cccc5cccc(-c6ccc7c(c6)c6ccccc6n7-c6ccc7ccccc7c6)c45)cc3)cc21]

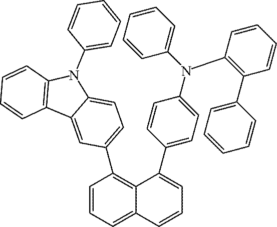 [SMILES: c1ccc(-c2ccccc2N(c2ccccc2)c2ccc(-c3cccc4cccc(-c5ccc6c(c5)c5ccccc5n6-c5ccccc5)c34)cc2)cc1]

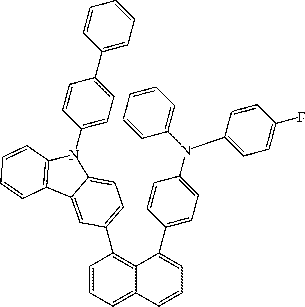 [SMILES: Fc1ccc(N(c2ccccc2)c2ccc(-c3cccc4cccc(-c5ccc6c(c5)c5ccccc5n6-c5ccc(-c6ccccc6)cc5)c34)cc2)cc1]

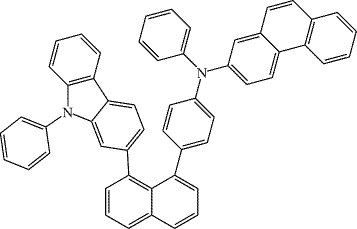 [SMILES: c1ccc(N(c2ccc(-c3cccc4cccc(-c5ccc6c7ccccc7n(-c7ccccc7)c6c5)c34)cc2)c2ccc3c(ccc4ccccc43)c2)cc1]

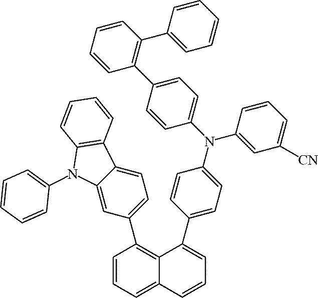 [SMILES: N#Cc1cccc(N(c2ccc(-c3ccccc3-c3ccccc3)cc2)c2ccc(-c3cccc4cccc(-c5ccc6c7ccccc7n(-c7ccccc7)c6c5)c34)cc2)c1]

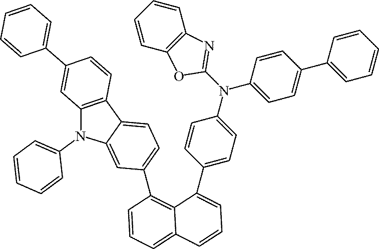 [SMILES: c1ccc(-c2ccc(N(c3ccc(-c4cccc5cccc(-c6ccc7c8ccc(-c9ccccc9)cc8n(-c8ccccc8)c7c6)c45)cc3)c3nc4ccccc4o3)cc2)cc1]

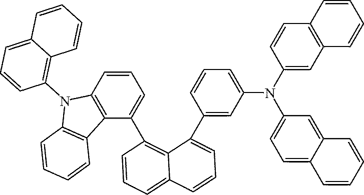 [SMILES: c1cc(-c2cccc3cccc(-c4cccc5c4c4ccccc4n5-c4cccc5ccccc45)c23)cc(N(c2ccc3ccccc3c2)c2ccc3ccccc3c2)c1]

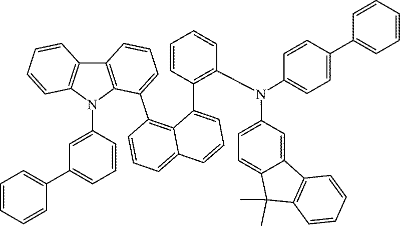 [SMILES: CC1(C)c2ccccc2-c2cc(N(c3ccc(-c4ccccc4)cc3)c3ccccc3-c3cccc4cccc(-c5cccc6c7ccccc7n(-c7cccc(-c8ccccc8)c7)c56)c34)ccc21]

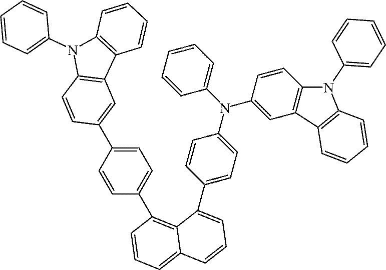 [SMILES: c1ccc(N(c2ccc(-c3cccc4cccc(-c5ccc(-c6ccc7c(c6)c6ccccc6n7-c6ccccc6)cc5)c34)cc2)c2ccc3c(c2)c2ccccc2n3-c2ccccc2)cc1]

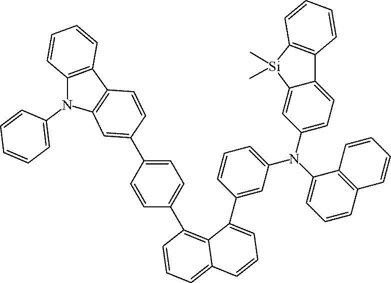 [SMILES: C[Si]1(C)c2ccccc2-c2ccc(N(c3cccc(-c4cccc5cccc(-c6ccc(-c7ccc8c9ccccc9n(-c9ccccc9)c8c7)cc6)c45)c3)c3cccc4ccccc34)cc21]

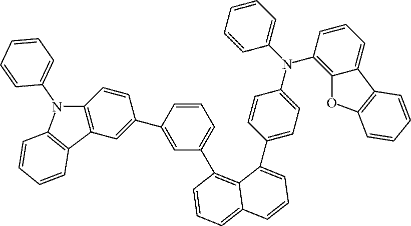

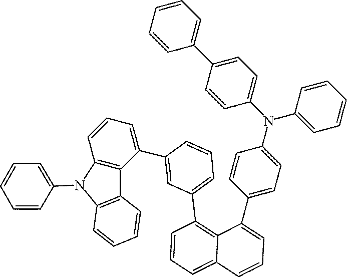 [SMILES: c1ccc(-c2ccc(N(c3ccccc3)c3ccc(-c4cccc5cccc(-c6cccc(-c7cccc8c7c7ccccc7n8-c7ccccc7)c6)c45)cc3)cc2)cc1]

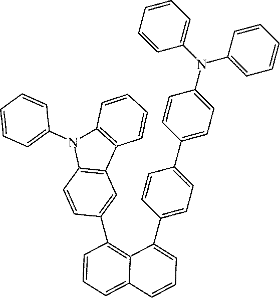 [SMILES: c1ccc(N(c2ccccc2)c2ccc(-c3ccc(-c4cccc5cccc(-c6ccc7c(c6)c6ccccc6n7-c6ccccc6)c45)cc3)cc2)cc1]

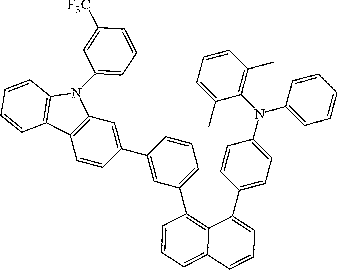 [SMILES: Cc1cccc(C)c1N(c1ccccc1)c1ccc(-c2cccc3cccc(-c4cccc(-c5ccc6c7ccccc7n(-c7cccc(C(F)(F)F)c7)c6c5)c4)c23)cc1]

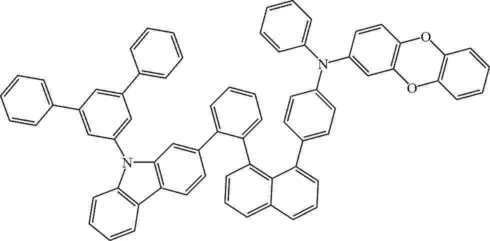 [SMILES: c1ccc(-c2cc(-c3ccccc3)cc(-n3c4ccccc4c4ccc(-c5ccccc5-c5cccc6cccc(-c7ccc(N(c8ccccc8)c8ccc9c(c8)Oc8ccccc8O9)cc7)c56)cc43)c2)cc1]

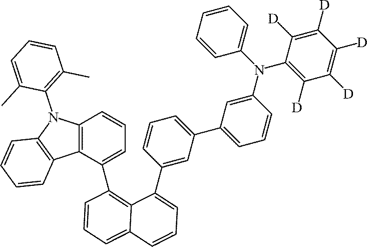 [SMILES: [2H]c1c([2H])c([2H])c(N(c2ccccc2)c2cccc(-c3cccc(-c4cccc5cccc(-c6cccc7c6c6ccccc6n7-c6c(C)cccc6C)c45)c3)c2)c([2H])c1[2H]]

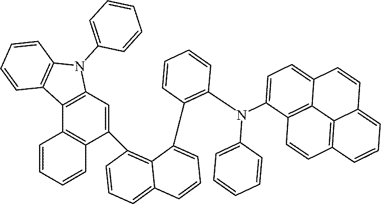 [SMILES: c1ccc(N(c2ccccc2-c2cccc3cccc(-c4cc5c(c6ccccc46)c4ccccc4n5-c4ccccc4)c23)c2ccc3ccc4cccc5ccc2c3c45)cc1]

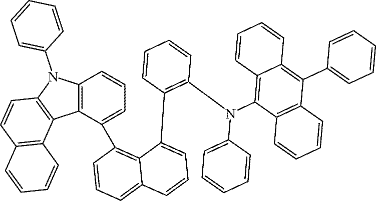 [SMILES: c1ccc(-c2c3ccccc3c(N(c3ccccc3)c3ccccc3-c3cccc4cccc(-c5cccc6c5c5c7ccccc7ccc5n6-c5ccccc5)c34)c3ccccc23)cc1]

13. An electronic element, comprising an anode and a cathode which are oppositely disposed, and a functional layer disposed between the anode and the cathode; wherein the functional layer comprises the nitrogen-containing compound of claim 1 .

14. The electronic element of claim 13, wherein the electronic element is an organic electroluminescent device or a photoelectric conversion device.

15. The electronic element of claim 13, wherein the functional layer comprises a hole transporting layer, and the hole transporting layer comprises the nitrogen-containing compound; and/or
the functional layer comprises a luminescence layer, and the luminescence layer comprises a host material and a guest material, wherein the host material comprises the nitrogen-containing compound.

16. An electronic device, comprising the electronic element of claim 13.

17. The nitrogen-containing compound of claim 3, wherein substituents in Ar₁, Ar₂, and Ar₃ are each independently selected from deuterium, fluorine, cyano, an aryl with 6 to 15 carbon atoms, a heteroaryl with 5 to 12 carbon atoms, an alkyl with 1 to 4 carbon atoms, a haloalkyl with 1 to 4 carbon atoms, a cycloalkyl with 5 to 10 carbon atoms, an alkoxy with 1 to 4 carbon atoms, an alkylthio with 1 to 4 carbon atoms, or a trialkylsilyl with 3 to 7 carbon atoms.

18. The nitrogen-containing compound of claim 7, wherein substituents in L₁, L₂ and L₃ are the same or different, and are each independently selected from deuterium, fluorine, cyano, phenyl, pyridyl, an alkyl with 1 to 4 carbon atoms, a haloalkyl with 1 to 4 carbon atoms, or a trialkylsilyl with 3 to 7 carbon atoms.
